(19) United States
(12) Patent Application Publication
(10) Pub. No.: US 2022/0372139 A1
(43) Pub. Date: Nov. 24, 2022

(54) ANTI-TREM-1 ANTIBODIES AND USES THEREOF

(71) Applicant: BRISTOL-MYERS SQUIBB COMPANY, Princeton, NJ (US)

(72) Inventors: Achal M. PASHINE, Mahwah, NJ (US); Derek A. HOLMES, Lawrencevillle, NJ (US); Clarence K. ZHANG, Newtown, PA (US); Dmitry OSTANIN, Newtown, PA (US)

(21) Appl. No.: 17/627,070

(22) PCT Filed: Jul. 15, 2020
(86) PCT No.: PCT/US2020/042169

§ 371 Date: Jan. 13, 2022

Related U.S. Application Data
(60) Provisional application No. 62/874,318, filed on Jul. 15, 2019.

(51) Int. Cl. C07K 16/28; A61P 1/00

(52) U.S. Cl. CPC C07K 16/2803; A61P 1/00; C07K 2317/565; A61K 2039/505

(57) ABSTRACT

Provided herein are methods of identifying subjects suitable for an anti-TREM-1 antibody (i.e., antagonistic anti-TREM-1 antibody) treatment comprising measuring an expression level of a TREM-1 associated gene. Also disclosed herein are methods of determining efficacy of an anti-TREM-1 antibody comprising measuring an expression level of a TREM-1 associated gene. Methods of identifying non-responder to a standard of care treatment and methods of treating a disease or disorder (e.g., inflammatory bowel disease) with an anti-TREM-1 antibody are also disclosed.

### CROSS-REFERENCE TO RELATED APPLICATIONS

This PCT application claims the priority benefit of U.S. Provisional Application No. 62/874,318, filed Jul. 15, 2019, which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name: 3338_1380000_SegListing.txt; Size: 486,499 bytes; and Date of Creation: Jul. 14, 2019) filed with the application is herein incorporated by reference in its entirety.

### BACKGROUND OF THE DISCLOSURE

TREM-1 is an activating receptor expressed on monocytes, macrophages, and neutrophils. By binding to its natural ligand peptidoglycan-recognition-protein (PGLYRP1), TREM-1 can help in the activation of these cells, resulting in the production of cytokines and other mediators that drive inflammation. Accordingly, TREM-1 mRNA and protein expression is up-regulated in many inflammatory diseases, including inflammatory bowel diseases (IBD), and TREM-1-positive cells accumulate at sites of inflammation, correlating with disease severity. See Bouchon et al., Nature 410:1103-1107 (2001); and Schenk et al., Clin Invest 117:3097-3106 (2007).

Inflammatory bowel disease (IBD) (e.g., ulcerative colitis (UC) and Crohn's disease (CD)) is a chronic disorder of the gastrointestinal tract characterized by inflammation of the intestines or colon. Symptoms of IBD can vary but generally include abdominal cramping, persistent diarrhea, and colorectal bleeding. IBD can be debilitating and can have life-threatening complications if left untreated.

There are no known cures for IBD. Current treatment options include drugs (e.g., anti-inflammatory agents, immunosuppressants, and antibiotics), nutrition supplements, and surgery. While such treatments can reduce the signs and symptoms of the disease, they generally have limited efficacy and/or adverse side effects. See, e.g., Martinez-Montiel, M, P., e: al., Clin Exp Gastroenterot 8:257-269 (2015); Cunliffe, R. N., et al., Aliment Pharmacol Ther 16(4):647-662 (2002). Moreover, IBD is difficult to diagnose, and the diagnostic methods available (e.g., blood/stool tests, x-rays, endoscopy colonoscopy), and/or tissue biopsies) are often very invasive. Therefore, IBD remains a major medical challenge worldwide, and there remains a need for new treatment and/or diagnostic options that are safer and more efficacious.

### SUMMARY OF THE DISCLOSURE

Provided herein is a method of identifying a subject suffering from a disease or disorder suitable for a treatment with an antagonistic anti-TREM-1 antibody. In certain embodiments, the method comprises measuring an expression level of a TREM-1 associated gene in a sample of the subject, wherein the TREM-1 associated gene comprises Nicotinamide phosphoribosyltransferase (NAMPT); dehydrogenase/reductase 9 (DHRS9); cyclin dependent kinase inhibitor 1A (CDKN1A); CD52 molecule (CD52); Myotubularin related protein 11 (MTMR11); EH domain containing 1 (EHD1); Solute carrier family 27 member 3 (SLC27A3); Interleukin 24 (IL24); Pim-2 proto-oncogene, serine/threonine kinase (P1M2); chitinase 3 like 1 (CHI3L1); Polypeptide N-acetylgalactosaminyltransferase 6 (GALNT6); Acyl-CoA thioesterase 7 (ACOT7); cytokine inducible SH2 containing protein (CISH); family with sequence similarity 129 member A (FAM129A); polo like kinase 3 (PLK3); major facilitator superfamily domain containing 12 (MFSD12); SCAR related lipid transfer domain containing 4 (STARD4); C-type lectin domain family 12 member A (CLEC12A); CD55 molecule (Cromer blood group) (CD55); Interferon lambda receptor 1 (IFNLR1), or combinations thereof.

In some embodiments, the method further comprises administering a therapeutically effective dose of the antagonistic anti-TREM-1 antibody to a subject who exhibits an increase in the expression level of the T REM-1 associated gene compared to a reference, wherein the reference comprises a subject not suffering from the disease or disorder (e.g., healthy subject).

Also provided herein is a method of treating a disease or disorder in a subject in need thereof, comprising administering a therapeutically effective dose of an antagonistic anti-TREM-1 antibody to the subject, wherein the subject exhibits an increase in an expression level of a TREM-1 associated gene, wherein the TREM-1 associated gene comprises Nicotinamide phosphoribosyltransferase (NAMPT); dehydrogenase/reductase 9 (DHRS9); cyclin dependent kinase inhibitor 1A (CDKN1A); CD52 molecule (CD52); Myotubularin related protein 11 (MTMR11); EH domain containing 1 (EHD1); Solute carrier family 27 member 3 (SLC27A3); Interleukin 24 (IL24); Pim-2 proto-oncogene, serine/threonine kinase (PIM2); chitinase 3 like 1 (CHI3L1); Polypeptide N-acetylgalactosaminyltransferase 6 (GALNT6); Acyl-CoA thioesterase 7 (ACOT7); cytokine inducible SH2 containing protein (CISH); family with sequence similarity 129 member A (FAM129A); polo like kinase 3 (PLK3); major facilitator superfamily domain containing 12 (MFSD12); StAR related lipid transfer domain containing 4 (STARD4); C-type lectin domain family 12 member A (CLEC12A); CD55 molecule (Cromer blood group) (CD55); Interferon lambda receptor 1 (IFNLR1), or combinations thereof.

In some embodiments, the subject was previously treated with a standard of care treatment for the disease or disorder and did not respond to the treatment.

In some embodiments, the standard of care treatment comprises an anti-TNF-α antibody. In certain embodiments, the anti-TNF-α antibody comprises infliximab (REMICADE®), certolizumab pegol (CIMZIA®), etanercept (ENBREL®), adalimumab (HUMIRA®), golimumab (SIMPONI®), or combinations thereof.

The present disclosure further provides a method method of identifying a non-responder to a standard of care treatment for a disease or disorder, comprising measuring an expression level of a TREM-1 associated gene in a sample of a subject who has received the standard of care treatment, wherein the subject exhibits an increase in the expression level of the TREM-1 associated gene and wherein the TREM-1 associated gene comprises Nicotinamide phosphoribosyltransferase (NAMPT); dehydrogenase/reductase 9 (DHRS9); cyclin dependent kinase inhibitor 1A (CDKN1A); CD52 molecule (CD52); Myotubularin related protein 11 (MTMR11); EH domain containing 1 (EHD1); Solute carrier family 27 member 3 (SLC27A3); Interleukin 24 (IL24); Pim-2 proto-oncogene, serine/threonine kinase (PIM2); chitinase 3 like 1 (CHI3L1); Polypeptide N-acetylgalactosaminyltransferase 6 (GALNT6); Acyl-CoA thioesterase 7 (ACOT7); cytokine inducible SH2 containing protein (CASH); family with sequence similarity 129 member A (FAM129A); polo like kinase 3 (PLK3); major facilitator superfamily domain containing 12 (MFSD12); StAR related lipid transfer domain containing 4 (STARD4); C-type lectin domain family 12 member A (CLEC12A); CD55 molecule (Cromer blood group) (CD55); Interferon lambda receptor 1 (IFNLR1), or combinations thereof.

In some embodiments, the standard of care treatment comprises an anti-TNF-α antibody (e.g., INFLIXIMAB®).

In some embodiments, the method of identifying a non responder to a standard of care treatment for a disease or disorder further comprises administering an additional therapeutic agent to a subject who has been identified as a non-responder to the standard of care treatment. In certain embodiments, the additional therapeutic agent comprises an antagonistic anti-TREM-1 antibody (e.g., those disclosed herein).

Provided herein is a method of determining efficacy of an antagonistic anti-TREM-1 antibody in treating a disease or disorder in a subject in need thereof, comprising administering the antagonistic anti-TREM-1 antibody to the subject and measuring an expression level of a TREM-1 associated gene in a sample of the subject, wherein the subject exhibits a decrease in the expression level of the TREM-1 associated gene after the administration, wherein the TREM-1 associated gene comprises Nicotinamide phosphoribosyltransferase (NAMPT); dehydrogenase/reductase 9 (DHRS9); cyclin dependent kinase inhibitor 1A (CDKN1A); CD52 molecule (CD52); Myotubularin related protein 11 (MTMR11); EH domain containing 1 (EHD1); Solute carrier family 27 member 3 (SLC27A3); Interleukin 24 (1L24); Pita-2 proto-oncogene, serine/threonine kinase (PIM2); chitinase 3 like 1 (CHI3L1); Polypeptide N-acetylgalactosaminyltransferase 6 (GALNT6); Acyl-CoA thioesterase 7 (ACOT7); cytokine inducible SH2 containing protein (CISH); family with sequence similarity 129 member A (FAM129A); polo like kinase 3 (PLK3); major facilitator superfamily, domain containing 12 (MFSD12); StAR related lipid transfer domain containing 4 (STARD4); C-type lectin domain family 12 member A (CLEC12A); CD55 molecule (Cromer blood group) (CD55); Interferon lambda receptor 1 (IFNLR1), or combinations thereof. In some embodiments, the subject is continued with the antagonistic anti-TREM-1 antibody treatment.

In some embodiments, any of the methods disclosed herein further comprises measuring one or more scores of a Baseline Mayo score, a Grade 2B Lamina Propria Neutrophil Infiltration score, and a fecal calprotectin level, prior to, concurrently, or after the measuring the expression level of the TREM-1 associated gene and/or administering the antagonistic anti-TREM-1 antibody.

In some embodiments, a subject (as applied to any of the methods disclosed herein) exhibits one or more of an increased Baseline Mayo score, an increased Grade 2B Lamina Propria Neutrophil Infiltration score, and an increased fecal calprotectin level, prior to the administration of the antagonistic anti-TREM-1 antibody.

In some embodiments, a subject exhibits an increased Baseline Mayo score by at least about 5%, at least about 10%, at least about 20%, at least about 30%, at least about 40%, at least about 50%, at least about 60%, at least about 70%, at least about 80%, at least about 90% or more compared to the reference.

In some embodiments, a subject exhibits a Baseline Mayo score greater than about 6, 7, 8, 9, 10, 11, or 12 prior to the administration.

In some embodiments, a subject exhibits an increased Grade 2B Lamina Propria Neutrophil Infiltration score by at least about 5%, at least about 10%, at least about 20%, at least about 30%, at least about 40%, at least about 50%, at least about 60%, at least about 70%, at least about 80%, at least about 90% or more compared to the reference.

In some embodiments, a subject exhibits a Grade 2B Lamina Propria Neutrophil Infiltration score greater than about 0, about 0.1, about 0.2, or about 0.3.

In some embodiments, a subject exhibits an increased fecal calprotectin level by at least about 5%, at least about 10%, at least about 20%, at least about 30%, at least about 40%, at least about 50%, at least about 60%, at least about 70%, at least about 80%, at least about 90% or more compared to the reference.

In some embodiments, a subject exhibits a fecal calprotectin level (μg/g of feces) greater than about 1.5 log 10, greater than about 2.0 log 10, greater than about 2.5 log 10, greater than about 3.0 log 10, or greater than about 3.5 log 10.

In some embodiments, administering an antagonistic anti-TREM-1 antibody (e.g., those disclosed herein) reduces the expression of the TREM-1 associated gene in a subject.

In some embodiments, administering an antagonistic anti-TREM-1 antibody reduces a Baseline Mayo score, Grade 2B Lamina Propria Neutrophil Infiltration score, and; or fecal calprotectin level of the subject. In certain embodiments, the Baseline Mayo score is decreased by at least about 5%, at least about 10%, at least about 20%, at least about 30%, at least about 40%, at least about 50%, at least about 60%, at least about 70%, at least about 80%, at least about 90% or more. In some embodiments, the Grade 2B Lamina Propria Neutrophil Infiltration score is decreased by at least about 5%, at least about 10%, at least about 20%, at least about 30%, at least about 40%, at least about 50%, at least about 60%, at least about 70%, at least about 80%, at least about 90% or more. In further embodiments, the fecal calprotectin level is decreased by at least about 5%, at least about 10%, at least about 20%, at least about 30%, at least about 40%, at least about 50%, at least about 60%, at least about 70%, at least about 80%, at least about 90% or more.

In some embodiments, the expression level of the TREE-1 associated gene is increased in the presence of a natural ligand for TREM-1 but not in the presence of an agonistic anti-TREM-1 antibody.

In some embodiments, a sample comprises a tissue, blood, serum, plasma, saliva, urine, or combinations thereof.

In some embodiments, a disease or disorder (as applied to any of the methods disclosed herein) is associated with increased degranulation, reactive oxygen species formation, and/or release of pro-inflammatory cytokines by neutrophils. In some embodiments, the disease or disorder is associated with activation of monocytes and/or increased production of inflammatory cytokines and chemokines by monocytes. In certain embodiments, the disease or disorder is associated with hypoxia. In further embodiments, the disease or disorder is associated with an increase in cell surface TREM-1 protein expression and/or an increase in level of soluble TREM-1 protein.

In some embodiments, a disease or disorder (as applied to any of the methods disclosed herein) comprises an inflammatory bowel disease (IBD), Crohn's disease (CD), ulcerative colitis (UC), irritable bowel syndrome, rheumatoid arthritis (RA), psoriasis, psoriatic arthritis, systemic lupus erythematosus (SLE), lupus nephritis, vasculitis, sepsis, systemic inflammatory response syndrome (SIRS), type I diabetes, Grave's disease, multiple sclerosis (MS), autoimmune myocarditis, Kawasaki disease, coronary artery disease, chronic obstructive pulmonary disease, interstitial lung disease, autoimmune thyroiditis, scleroderma, systemic sclerosis, osteoarthritis, atopic dermatitis, vitiligo, graft versus host disease, Sjogrens's syndrome, autoimmune nephritis, Goodpasture's syndrome, chronic inflammatory demyelinating polyneuropathy, allergy, asthma, other autoimmune diseases that are a result of either acute or chronic inflammation, chronic kidney disease, or combinations thereof. In certain embodiments, the disease or disorder is inflammatory bowel disease. In some embodiments, the inflammatory bowel disease comprises Crohn's disease and ulcerative colitis.

In some embodiments, an anti-TREM-1 antibody (e.g., those disclosed herein) comprises a heavy chain CDR1, CDR2, and CDR3 and a light chain CDR1, CDR2, and CDR3, wherein the heavy chain CDR3 comprises DMGIRRQFAY (SEQ ID NO: 19) or DMGIRRQFAY (SEQ ID NO: 19) except one or two substitutions. In certain embodiments, the heavy chain CDR3 comprises DQGIRRQFAY (SEQ ID NO: 72).

In some embodiments, an anti-TREM-1 antibody comprises a heavy chain CDR1, CDR2, and CDR3 and a light chain CDR1, CDR2, and CDR3, wherein the heavy chain CDR2 comprises RIRTKSSNYATYYAASVKG (SEQ ID NO: 18) or RIRTKSSNYATYYAASVKG (SEQ ID NO: 18) except one or two substitutions.

In some embodiments, an anti-TREM-1 antibody comprises a heavy chain CDR1, CDR2, and CDR3 and a light chain CDR1, CDR2, and CDR3, wherein the heavy chain CDR1 comprises TYAMH (SEQ ID NO: 17) or TYAMH (SEQ ID NO: 17) except one or two substitutions.

In some embodiments, an anti-TREM-1 antibody comprises a heavy chain CDR1, CDR2, and CDR3 and a light chain CDR1. CDR2, and CDR3, wherein the light chain CDR1 comprises RASQSVDTFDYSFLH (SEQ ID NO: 24) or RASQSVDTFDYSFLH (SEQ ID NO: 24) except one or two substitutions.

In some embodiments, an anti-TREM-1 antibody comprises a heavy chain CDR1, CDR2, and CDR3 and a light chain CDR1, CDR2, and CDR3, wherein the light chain CDR2 comprises RASNLES (SEQ ID NO: 21) or RASNLES (SEQ ID NO: 21) except one or two substitutions.

In some embodiments, an anti-TREM-1 antibody comprises a heavy chain CDR1, CDR2, and CDR3 and a light chain CDR1, CDR2, and CDR3, wherein the light chain CDR3 comprises QQSNQDPYT (SEQ ID NO: 25) or QQSNQDPYT (SEQ ID NO: 25) except one or two substitutions.

In some embodiments, an anti-TREM-1 antibody comprises a heavy chain variable region (VH) and a light chain variable region (VL), wherein the VH comprises the amino acid sequence set forth as SEQ ID NO: 0.15 or 26-29 and the VL comprises the amino acid sequence set forth as SEQ ID NO: 23.

In some embodiments, an anti-TREM-1 antibody comprises a heavy chain (HC) and a light chain (LC), wherein the FIC comprises the amino acid sequence set forth as SEQ ID NO: 30, 31, 32, or 33. In certain embodiments, the LC comprises the amino acid sequence set forth as SEQ ID NO: 34.

In some embodiments, an anti-TREM-1 antibody comprises a heavy chain CDR1, CDR2, and CDR3 and a light chain CDR1, CDR2, and CDR3, wherein (a) the heavy chain CDR1, CDR2, and CDR3 comprises the amino acid sequence set forth as SEQ NOs: 61, 62, and 63, respectively, and the light chain CDR1, CDR2, and CDR3 comprises the amino acid sequence set forth as SEQ ID NOs: 64, 65, and 66, respectively; (h) the heavy chain CDR1, CDR2, and CDR3 comprises the amino acid sequence set forth as SEQ ID NOs: 67, 68, and 69, respectively, and the light chain CDR1, CDR2, and CDR3 comprises the amino acid sequence set forth as SEQ ID NOs: 70; 71, and 72, respectively; (c) the heavy chain CDR1, CDR2, and CDR3 comprises the amino acid sequence set forth as SEQ ID NOs: 67, 68, and 69, respectively, and the light chain CDR1, CDR2, and CDR3 comprises the amino acid sequence set forth as SEQ ID NOs: 64, 65, and 73, respectively; (d) the heavy chain CDR1, CDR2, and CDR3 comprises the amino acid sequence set forth as SEQ ID NOs: 74, 75, and 76, respectively, and the light chain CDR1, CDR2, and CDR3 comprises the amino acid sequence set forth as SEQ ID NOs: 70, 77, and 78, respectively; (e) the heavy chain CDR1, CDR2, and CDR3 comprises the amino acid sequence set forth as SEQ ID NOs: 79, 80, and 81, respectively, and the light chain CDR1, CDR2, and CDR3 comprises the amino acid sequence set forth as SEQ ID NOs: 70, 71, and 72, respectively; (f) the heavy chain CDR1, CDR2, and CDR3 comprises the amino acid sequence set forth as SEQ ID NOs: 159, 160, and 161, respectively, and the light chain CDR1, CDR2, and CDR3 comprises the amino acid sequence set forth as SEQ ID NOs: 70, 71, and 162; respectively; or (g) the heavy chain CDR1, CDR2, and CDR3 comprises the amino acid sequence set forth as SEQ ID NOs: 159, 160, and 161, respectively, and the light chain CDR1, CDR2, and CDR3 comprises the amino acid sequence set forth as SEQ ID NOs: 70, 71, and 133, respectively.

In some embodiments, an anti-TREM-1 antibody used for the methods disclosed herein comprises a heavy chain variable region (VH) and a light chain variable region (VL), wherein the VH comprises an amino acid sequence set forth in SEQ ID NO: 53, 55, 58, 60, or 153 and wherein the VL comprises an amino acid sequence set forth in SEQ ID NO: 54, 56; 57, 59, 154, or 155.

In some embodiments, an anti-TREM-1 antibody further comprises a heavy chain (HC) constant region and a light chain (LC) constant region, wherein the HC constant region comprises the amino acid sequence set forth as SEQ ID NO: 48, SEQ ID NO: 47, SEQ ID NO: 11, or SEQ ID NO: 12. In certain embodiments, the LC constant region comprises the amino acid sequence set forth as SEQ ID NO: 35.

In some embodiments, an anti-TREM-1 antibody disclosed herein comprises a heavy chain CDR1, CDR2, and CDR3 and a light chain CDR1, CDR2, and CDR3, wherein (a) the heavy chain CDR1 comprises amino acids 31 to 35 (TYAMH) of SEQ NO: 13; (b) the heavy chain CDR2 comprises amino acids 50 to 68 (RIRTKSSNYATYYAASVKG) of SEQ ID NO: 13; (c) the heavy chain CDR3 comprises amino acids 101 to 110 (DMGQRRQFAY) of SEQ ID NO: 13; (d) the light chain CDR1 comprises amino acids 24 to 38 (RASESVDTFDYSFLH) of SEQ ID NO: 14; (e) the light chain CDR2 comprises amino acids 54 to 60 (RASNLES) of SEQ ID NO: 14; and/or (f) the chain CDR3 comprises amino acids 93 to 101 (QQSNEDPYT) of SEQ ID NO: 14.

In some embodiments, an anti-TREM-1 antibody comprises a heavy chain variable region (VH) and a light chain variable region (VL), wherein the VH comprises amino acids 1 to 121 of SEQ ID NO: 13 and wherein the VL comprises amino acids 1 to 111 of SEQ ID NO: 14.

In some embodiments, an anti-TREM-1 antibody comprises a heavy chain (HC) and a light chain (LC), wherein the HC comprises the amino acid sequence set forth as SEQ ID NO: 13 and wherein the LC comprises the amino acid sequence set forth as SEQ ID NO: 14.

### BRIEF DESCRIPTION OF THE DRAWINGS/FIGURES

FIGS. 1A-1D provide comparison of TNF-α production by human monocytes after stimulation with peptidoglycan recognition protein-1 (PGLYRP1) and/or peptidoglycan (PGN). The different stimulation conditions were as follows: (i) unstimulated (“Unstim”); (ii) PGLYRP1 alone (“PGRP”); (iii) PGN alone (“PGN”); and (iv) both PGLYRP1 and PGN (“PGRP+PGN”). In FIGS. 1A, 1B, and 1C, PGN was derived from Staphylococcus aureus (PGN-SA). Escherichia cull (PGN-EK), and Bacillus subtilus (PGN-BS), respectively. In FIG. 1D. PGN that lacked Toll-like receptor 2 (TLR2) binding (PGN-ECndss) was used to stimulate the monocytes. In each of FIGS. 1A-ID. TNF-α values are provided as means±s.e.m.

FIG. 2 provides a principal component analysis plot showing the relatedness of all monocyte samples after 24-hour stimulation under six different conditions. The stimulation conditions were as follows: (i) unstimulated (square), (ii) PGN-ECndss alone, (“PGN-EC”; circle) (iii) PGN-ECndss PGLYRP1 (“PGN+PGRP”; triangle), (iv) PGN-ECndss+PGLYRP1+isotype antibody (“PGN+PGRP+isotype”; cross), (v) PGN-ECndss+PGLYRP1+anti-TREM1 blocking antibody (“PGN PGRP+Trem1”; star), and (vi) PGLYRP1 only (“P+L”; diamond).

FIG. 3 provides a scatter plot demonstrating the specificity of the genes induced after TREM-1 ligand stimulation to the TREM-1 signaling pathway. The x-axis shows the log 2 fold change in gene expression after stimulation with PGLYRP1+PGN-ECndss vs PGN-ECndss (“P+L vs P”). The y-axis shows the log 2 fold change in gene expression after anti-TREM-1 inhibition with the anti-TREM-1 antibody vs an isotype control (“P+L+aTREM vs P+L+iso”). The diagonal line represents the reference line indicating where the points should fall if they come from populations with the same distribution. Genes with increased expression after PG-N-ECndss+PGLYRP1 (“P+L”) treatment compared to PGN-ECndss alone (“P”) are shown in dark gray. Genes with decreased expression after treatment with PGN-ECndss+PGLYRP1+anti-TREM1 blocking antibody (“P+L+aTREM”) compared to PGN-ECndss+PGLYRP1+isotype antibody control (“P+L+Iso”) are shown in light gray.

FIGS. 4A and 4B provide principal component analysis plot showing the relatedness of neutrophils cultured in different stimulation conditions. The stimulation conditions for both FIGS. 4A and 4B were as follows: (i) unstimulated (square), (ii) PGN-ECndss alone (“PGN-EC”; circle), (iii) PGN-ECndss+PGLYRN (“PGN+PGRP”; triangle), (iv) PGN-ECndss+PGLYRP1 isotype control antibody (“PGN+PGRP+isotype”; cross), (v) PGN-ECndss+PGLYRP1+anti-TREM1 blocking antibody (“PGN PGRP+Trem1”; star), and (vi) PGLYRP1 only (“PL”; diamond). FIG. 4A provides the results after 6 hour stimulation. FIG. 4B provides the results from 4 different donors: D249 (square), D254 (circle), D274 (triangle), and D299 (diamond).

FIG. 5 provides comparison of the overlap in gene expression profiles in monocytes stimulated with different TREM-1 agonists. FIG. 5 shows a scatter plot comparing the log 2 fold change of the TREM-1 natural ligand (PGLYRP1 PGN-ECndss vs PGN-ECndss alone, x-axis) against the log 2 fold change of the agonistic anti-TREM1 antibody MAB1278 (agTREM1 vs isotype antibody). The dark gray dots represent genes that changed expression level after stimulation with the TREM-1 natural ligand. The light gray dots represent genes that changed expression level after stimulation with the agonistic anti-TREM1 antibody. The black lines represent the linear regression for the genes that changed expression levels for each of the TREM-1 agonists.

FIGS. 6A-6F provide comparison of cytokine levels produced by monocytes after TREM-1 stimulation. TREM-1 expressing monocytes were stimulated with either PGN alone (“PGN”) or PGN in combination with PGLYRP1 (“PGRP+PGN”). Unstimulated monocytes (“Unstim”) were used as negative control. To confirm that the cytokines produced were specific to TREM-1 activation, some of the monocytes were stimulated with PGN and PGLYRP1 in combination with an antagonist anti-TREM-1 antibody (“PGRP+PGN+anti-TREM1”). FIGS. 6A, 6B, and 6C show the protein levels of CCL20, IL-1β, and IL-12p40 produced by the monocytes, respectively. FIGS. 6D, 6E, and 6F show the gene expression levels of CCL20, IL-1β, and IL-23β, respectively. In FIGS. 6D-6F, the gene expression levels are shown as fold increase compared to the expression level in an unstimulated monocyte. Data are shown as means±s.e.m.

FIG. 7 shows expression of the TREM-1 gene signature profile in lesional (“DIS”) or non-lesional (“NOR”) colon biopsies from ulcerative colitis patients. The TREM-1 gene signature profile is shown as ssGSEA score, which is a rank based score summarizing the collective expression enrichment for all genes in TREM-1 module. See Example 2. The ulcerative colitis patients are from a clinical phase 2 trial evaluating the efficacy of an anti-IP10 antibody (ClinicalTrials.gov identifier NCT00656890). Data are shown both individually and as mean±s.e.m. The box plot heights represent the 25% and 75% quantiles along with median in the center.

FIG. 8 shows the correlation between TREM-1 gene signature profile and TREM-1 mRNA expression levels in lesional (“DIS”) or non-lesional (“NOR”) colon biopsies from ulcerative colitis patients. The TREM-1 gene signature profile is shown as ssGSEA score, which is a rank based score summarizing the collective expression enrichment for all genes in TREM-1 module. See Example 2. TREM-1 mRNA expression level is shown on the X-axis and TREM-1 signature score (based on the TREM 180 gene module from the monocytes, see Example 2) is shown on the Y-axis. The diagonal lines represent the best fit linear regression.

FIG. 9 shows the TREM-1 gene signature profile in ulcerative colitis patients that did or did not have prior standard of care treatment (i.e., anti-TNF treatment or oral corticosteroid use). Data was derived from colon biopsies of ulcerative colitis patients in a clinical phase 2 trial evaluating the efficacy of an anti-IP10 antibody (ClinicalTrials.gov identifier NCT00656890). Patients with a prior history of anti-TNF therapy were considered non-responders/inadequate responders (“anti-TNF IR/NR”). Patients without a record of anti-TNF were considered anti-TNF naïve (“anti-TNF Naive”). In each of the anti-TNF therapy groups, patients that received oral corticosteroids (“\textcircled{1}”; “YES”) and patients that did not receive oral corticosteroids (“\textcircled{2}”; “NO”) are shown. The TREM-1 gene signature profile is shown as ssGSEA score.

FIG. 10 shows the TREM-1 gene signature profile in patients with ulcerative colitis (UC) or Crohn's disease (CD) after treatment with Infliximab. Data was derived from a public dataset (GSEI6879) of colon biopsies from inflammatory bowel disease patients from baseline and 4-6 weeks post-Infliximab treatment compared to non-inflammatory bowel disease colon biopsies. For each patient, a ssGSEA score was calculated, which is a rank based score summarizing the collective expression enrichment for all genes in the TREM-1 module (shown on the y-axis). In both the UC and CD groups, patients were categorized as either a responder to Infliximab (“TNF responder”) or a non-responder (“TNF non-responder”). Then, the ssGSEA score of the patient before (light gray) and after (dark gray) treatment is shown. Data are shown both individually and as mean±s.e.m. The box plot heights represent the 25% and 75% quantiles along with median in the center.

FIGS. 11A and 11B show the ulcerative colitis (UC)-specific TREM-1 gene signature profile among different UC patients. The criteria used to generate the UC-specific TREM-1 signature, which contains 38 different genes, are provided in Example 7. FIG. 11A is a heatmap showing the expression pattern of each individual genes within the UC-specific TREM-1 signature in lesional biopsies from different UC patients. Y-axis shows the individual genes and x-axis shows each patient. Both the Baseline Partial Mayo and Geboes Global JS scores are provided for each of the patients. The brackets show the hierarchical clustering of the patient genes and how the patients cluster together based on the manner of expression of the 38 genes. FIG. 11B provides a histogram plot of the distribution of the UC-specificTREM-1 gene signature score (shown on the x-axis) among the UC patients. The black line represents the best fit curve.

FIGS. 12A-12C show the relationship between TREM-1 signature score and different proposed surrogate biomarkers of ulcerative colitis. FIG. 12A shows a comparison of the UC specific TREM-1 signature score (y-axis) in relation to a patient's baseline Mayo Score (x-axis). FIG. 12B shows the TREM-1 signature score (y-axis) compared to Geboes Grade 2B:lamina propria (LP) neutrophil infiltration (one measurement of the Geboes grading system) score (x-axis). For the LP neutrophil infiltration score, the scores shown are as follows: 0.0—no increase; 0.1—mild but unequivocal increase; 0.2—moderate increase; 0.3—marked increase). FIG. 12C shows the TREM-1 signature score (y-axis) compared to fecal calprotectin levels (shown as log 10, x-axis). In FIGS. 12A and 12C, the diagonal line represents best fit linear regression.

### DETAILED DESCRIPTION OF DISCLOSURE

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety:

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “triggering receptor expressed on myeloid cells 1” (also known as TREM1, TREM-1, and CD354) refers to a receptor that is expressed on monocytes, macrophages, and neutrophils. Primary ligand for TREM-1 include peptidoglycan-recognition-protein 1 (PGLYRP1), which belongs to a family of peptidoglycan (PGN) binding proteins (PGRPs). When activated, TREM-1 associates with the ITAM-containing signaling adaptor protein, DAP12. Downstream signaling can include activation of the NFAT transcription factor, causing an up-regulation of pro-inflammatory cytokine production. As used herein, the term “TREM-1” includes any variants or isoforms of TREM-1.

Three isoforms of human TREM-1 have been identified isoform 1 (Accession No. NP_061113.1; SEQ ID NO: 1) consists of 234 amino acids and represents the canonical sequence. Isoform 2 (Accession No. NP_001229518.1; SEQ ID NO: 2) consists of 225 amino acids and differ from the canonical sequence at amino acid residues 201-234. The amino acid residues encode part of the transmembrane domain and the cytoplasmic domain. Isoform 3 (Accession No. NP_001229519; SEQ ID NO: 3) consists of 150 amino acids, and is soluble. It lacks amino acid residues 151-234, which encode the transmembrane domain, the cytoplasmic domain, and part of the extracellular domain. The amino acid residues 138-150 also differ from the canonical sequence described above.

Below are the amino acid sequences of the three known human TREM-1 isoforms.

(A) Human TREM-1 isoform 1 (Accession No. NP_061113.1; SEQ ID NO: 1; encoded by the nucleotide sequence having Accession No. NM_018643; SEQ ID NO: 4):

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGPSGTPGSNENSTQN
VYKIPPTTTKALCPLYTSPRTVTQAPPKSTADVSTPDSEINLTNVTDIIR
VPVFNIVILLAGGEDSKSLVFSVLFAVTLRSEVP
(signal sequence is underlined);

(B) Human TREM-1 isoform 2 (Accession No, NP_001229518.1; SEQ NO: 2; encoded by the nucleotide sequence having Accession No. NM_001242589; SEQ ID NO: 5):

MRKTRLWGLLWMDEVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFSGTPGSNENSTQN
VYKIPPTTTKALCPLYTSPRTVTQAPPKSTADVSTPDSEINLTNVTDIIR
YSFQVPGPLVWTLSPLFPSLCAERM
(signal sequence is underlined);

(C) Human TREM-1 isoform 3 (Accession No. NP_001229519; SEQ ID NO: 3; encoded by the nucleotide sequence having Accession No. NM_001242590; SEQ ID NO: 6):

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFRCSTLSPSWLVDS
(signal sequence is underlined).

Cynomolgus TREM-1 protein (Accession No. XP_001082517; SEQ ID NO: 7) is predicted to have the following amino acid sequence:

MRKTRLWGLLWMLFVSELRATTELTEEKYEYKEGQTLEVKCDYALEKYAN
SRKAWQKMEGKMPKILAKTERPSENSHPVQVGRITLEDYPDHGLLQVQMT
NLQVEDSGLYQCVIYQHPKESHVLFNPICLVVTKGSSGTPGSSENSTQNV
YRTPSTTAKALGPRYTSPRTVTQAPPESTVWSTPGSEINLTNVTDIIRVP
VFNIVIIVAGGFLSKSLVFSVLFAVTLRSEGP
(signal sequence is underlined).

As used herein, the terms “peptidoglycan-recognition-protein 1” and “PGLYRP1” refer to the natural ligand for TREM-1 protein. PGLYRP1, which is a highly conserved, 196 amino acid long protein consisting of a signal peptide and a peptidoglycan binding domain, is expressed in neutrophils and released upon their activation. The amino acid sequence of PGLYRP1 (Accession No. NP_005082.1; SEQ ID NO: 8) is provided below:

MSRRSMLLAWALPSLLRLGAAQETEDPACCSPIVPRNEWKALASECAQHL
SLPLRYVVVSHTAGSSCNTPASCQQQARNVQHYHMKTLGWCDVGYNFLIG
EDGLVYEGRGWNFTGAHSGHLWNPMSIGISFMGNYMDRVPTPQAIRAAQG
LLACGVAQGALRSNYVLKGHRDVQRTLSPGNQLYHLIQNWPHYRSP
(signal sequence is underlined).

The term “inflammatory bowel disease” or “TBD,” as used herein, refers to a group of disorders that cause the intestines and/or the colon to become inflamed, generally manifested with symptoms including, but not limited to, abdominal cramps and pain, diarrhea, weight loss, and intestinal bleeding. The main forms of IBD are ulcerative colitis (UC) and Crohn's disease (CD).

“Ulcerative colitis” is a chronic, episodic, inflammatory disease of the large intestine and rectum characterized by bloody diarrhea. Ulcerative colitis is characterized by chronic inflammation in the colonic mucosa and can be categorized according to location: “proctitis” involves only the rectum, “proctosigmoiditis” affects the rectum and sigmoid colon, “left-sided colitis” encompasses the entire left side of the large intestine, “pancolitis” inflames the entire colon.

“Crohn's disease” (also called “regional enteritis”) is a chronic autoimmune disease that can affect any part of the gastrointestinal tract but most commonly occurs in the ileum (the area where the small and large intestine meet). Crohn's disease, in contrast to ulcerative colitis, is characterized by chronic inflammation extending through all layers of the intestinal wall and involving the mesentery as well as regional lymph nodes. Whether or not the small bowel or colon is involved, the basic pathologic process is the same.

The severity of a subject's HID can be determined based on various methods known in the art, which generally rely upon a combination of patient characteristics. Non-limiting examples of such methods include disease activity index (DAI)/Mayo Score, Geboes Score, Truelove & Wills' Severity Index, St Marks Index, Clinical Activity Index (CAI), Activity Index (AI), Simple Clinical Colitis Index (SCCAI), Ulcerative Colitis Clinical Score (UCCS), Crohn's Disease Activity Index (CDAI), Inflammatory Bowel Disease Questionnaire (IBDQ), Health-related quality of life (FIRQL), and Harvey-Bradshaw Index (RBI). See Source: US20180147265A1 and Cooney, R. M., et al., Trials 8:17 (2007) and Jauregui-Amezaga, A., et al., J Crohns Colitis 11(3):305-313 (2017), each of which is hereby incorporated in its entirety.

As used herein, the term “Mayo Score” or “Mayo Scoring System” refers to a 12-point composite index that is composed of inputs from the patient and from the person treating the patient (e.g., physician). See US20160324919A1 and Schroeder et al., Engl J Med 317(26):1625-29 (1987). Each sub-score of the Mayo system ranges from 0 to 3 depending upon the severity. The sum of the individual sub-scores provides the total Mayo score. See Table 1 (below).

TABLE 1
Mayo Scoring System

| | Normal | Mild | Moderate | Severe |
|---|---|---|---|---|
| | (Score = 0) | (Score = 1) | (Score = 2) | (Score = 3) |
| Rectal | None | Visible blood | Visible blood | Passing blood |
| Bleeding | | with stool less | with stool half | alone |
| | | than half the | of the time or | |
| | | time | more | |
| Stool | Normal^{a} | 1-2 stools/day | 3-4 stools/day | >4 stools/day |
| Frequency | | more than | more than | than normal |
| | | normal | normal | |
| Mucosal | Normal or | Mild disease | Moderate | Severe disease |
| Appearance/ | inactive | (erythema, | disease | (spontaneous |
| Endoscopy | disease | decreased | (marked | bleeding, |
| | | vascular | erythema, | ulceration) |
| | | pattern, mild | absent | |
| | | friability) | vascular | |
| | | | pattern, | |
| | | | friability, | |
| | | | erosions) | |
| Physician's | Normal | Mild | Moderate | Severe |
| Global | | | | |
| Assessment^{b} | | | | |

^{a}“Normal” stool frequency refers to the average number of stools per day when the patient is in remission.
^{b}Physician's Global Assessment is based on rectal bleeding, stool frequency, mucosal appearance, patient reported abdominal pain, the patient's general sense of well-being, and physical examination findings.

As used herein, the term Geboes Score refers to a histopathologic scoring system that utilizes a 6-point grading system (0-5) to measure disease activity based on architectural changes, chronic inflammatory infiltrate, lamina propria neutrophils and, eosinophils, neutrophils in epithelium, crypt destruction, and erosions and ulcerations. See WO2017095875A1 and Geboes, K., et al., Gut 47(3):404-9 (2000), which are hereby incorporated in its entirety. Higher grades indicate more severe disease activity. See Table 2 (below).

TABLE 2
Geboes Scoring System

| Grade 0 | Structural ( Architectural Changes) |
|---|---|
| Subgrades | |
| 0.0 | No abnormality |
| 0.1 | Mild abnormality |
| 0.2 | Mild or moderate diffuse or multifocal abnormalities |
| 0.3 | Severe diffuse or multifocal abnormalities |
| Grade 1 | Chronic Inflammatory Infiltrates |
| Subgrades | |
| 1.0 | No increase |
| 1.1 | Mild but unequivocal increase |
| 1.2 | Moderate increase |
| 1.3 | Marked increase |
| Grade 2 | Lamina Propria Neutrophils and Eosinophils |
| 2A Eosinophils | |
| 2A.0 | No increase |
| 2A.1 | Mild but unequivocal increase |
| 2A.2 | Moderate increase |
| 2A.3 | Marked increase |
| 2B Neutrophils | |
| 2B.0 | None |
| 2B.1 | Mild but unequivocal increase |
| 2B.2 | Moderate increase |
| 2B.3 | Marked increase |
| Grade 3 | Neutrophils in Epithelium |
| 3.0 | None |
| 3.1 | <5% crypts involved |
| 3.2 | <50% crypts involved |
| 3.3 | >50% crypts involved |
| Grade 4 | Crypt Destruction |
| 4.0 | None |
| 4.1 | Probable-local excess of neutrophils in part of crypt |
| 4.2 | Probable-marked attenuati on |
| 4.3 | Unequivocal crypt destraction |
| Grade 5 | Erosion or Ulceration |
| 5.0 | No erosion, ulceration, or granulation tissue |
| 5.1 | Recovering epithelium + adjacent inflammation |
| 5.2 | Probable erosion-focally stripped |
| 5.3 | Unequivocal erosion |
| 5.4 | Ulcer or granulation tissue |

The term “Grade 2B Lamina Propria Neutrophil Infiltration score,” as used herein, refers to one of the grades in the Geboes Scoring System (see Table 2, above).

As used herein, the term “fecal calprotectin” refers to a biochemical measurement of the protein calprotectin in the stool. Calprotectin is a member of the S100 calcium-binding protein family and exists as a heterodimer of S100A8 and S100A9 proteins. Calprotectins are largely produced by neutrophils, and elevated levels of calprotectin have been used as a diagnostic marker for diseases, such as IBD, coeliac disease, infectious colitis, necrotizing enterocolitis, intestinal cystic fibrosis, and colorectal cancer. See Konikoff, M. R., et al., Inflammm Bowel Dis 12(6):524-34 (2006). In some embodiments, reference fecal calprotectin levels (in of feces) are as follows: (i) normal (≤50.0), (ii) borderline (50.1-120.0), and (iii) abnormal (≥120.1). Fecal calprotectin levels can be determined by any methods known in the art (e.g., ELISA, immunofluorescence assay). See Labaere, D., et al., United European Gastroenterol J 2(1): 30-37 (2014).

The term “antibody” as used herein refers to a protein, derived from a germline immunoglobulin sequence, which is capable of specifically binding to an antigen (TREM-1) or a portion thereof. The term includes full length antibodies of any class or isotype (that is, IgA, IgE, IgG, IgM and; or IgY) and any single chain or fragment thereof. An antibody that specifically binds to an antigen, or portion thereof, may bind exclusively to that antigen, or portion thereof, or it may bind to a limited number of homologous antigens, or portions thereof. Full-length antibodies usually comprise at least four polypeptide chains: two heavy (H) chains and two light (L) chains that are interconnected by disulfide bonds. One immunoglobulin sub-class of particular pharmaceutical interest is the IgG family. In humans, the IgG class may be sub-divided into 4 sub-classes: IgG1, IgG2, IgG3 and IgG4, based on the sequence of their heavy chain constant regions. The light chains can be divided into two types, kappa and lambda, based on differences in their sequence composition. IgG molecules are composed of two heavy chains, interlinked by two or more disulfide bonds, and two light chains, each attached to a heavy chain by a disulfide bond. A heavy chain may comprise a heavy chain variable region (VH) and up to three heavy chain constant (CH) regions: CH1, CH2 and CH3. A light chain may comprise a light chain variable region (VL) and a light chain constant region (CL). VH and VL regions can be further subdivided into regions of hypervariability; termed complementarity determining regions (CDRs), interspersed with regions that are more conserved, termed framework regions (FR). VR and VL regions are typically composed of three CDRs and four FRs, arranged from amino-terminus to carboxy-terminus in the following order: FR1, CDR1, FR2, CDR2, FR3, CDR3, FR4. The hypervariable regions of the heavy and light chains form a binding domain that is capable of interacting with an antigen, whilst the constant region of an antibody may mediate binding of the immunoglobulin to host tissues or factors, including but not limited to various cells of the immune system (effector cells), Fc receptors and the first component (C1q) of the classical complement system. Antibodies of the current invention may be isolated. The term “isolated antibody” refers to an antibody that has been separated and/or recovered from (an)other component(s) in the environment in which it was produced and; or that has been purified from a mixture of components present in the environment in which it was produced. Certain antigen-binding fragments of antibodies may be suitable in the context of the current invention, as it has been shown that the antigen-binding function of an antibody can be performed by fragments of a full-length antibody.

The term “antigen-binding portion” of an antibody refers to one or more fragment(s) of an antibody that retain the ability to specifically bind to an antigen; such as TREM-1, as described herein. Examples of antigen-binding fragments include Fab, Fab′, F(ab)2, F(ab′)2, F(ah)S, Fv (typically the VL and VH domains of a single arm of an antibody), single-chain Fv (scFv; see, e.g., Bird et at., Science 242:42S-426 (1988); Huston et al., PNAS 85: 5879-5883 (1988)), dsFv, Fd (typically the YH and CH1 domain), and dAb (typically a VH domain) fragments; VH, VL, VhH, and V-NAR domains; monovalent molecules comprising a single VIII and a single VL chain; minibodies, diabodies, triabodies, tetrabodies, and kappa bodies (see, e.g., Ill et al., Protein Eng 10:949-57 (1997)); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2S:1126-1136 (2005); International Publ. No. WO 2005/040219, and U.S. Publ. Nos. 2005/0238646 and 2002/0161201 These antibody fragments may be obtained using conventional techniques known to those of skill in the art, and the fragments may be screened for utility in the same manner as intact antibodies.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The anti-TREM-1 antibodies described herein can include amino acid residues not encoded by human germline immunoglobulin sequences (e.g., mutations introduced by random or site-specific mutagenesis in vitro or by somatic imitation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to a human/non-human chimeric antibody that contains one or more sequences (CDR regions or parts thereof) that are derived from a non-human immunoglobulin. A humanized antibody is, thus, a human immunoglobulin (recipient antibody) in which at least residues from a hyper-variable region of the recipient are replaced by residues from a hyper-variable region of an antibody from a non-human species (donor antibody) such as from a mouse, rat, rabbit or non-human primate, which have the desired specificity, affinity, sequence composition and functionality. In some instances, FR residues of the human immunoglobulin are replaced by corresponding non-human residues. An example of such a modification is the introduction of one or more so-called back-mutations, which are typically amino acid residues derived from the donor antibody. Humanization of an antibody may be carried out using recombinant techniques known to the person skilled in the art (see, e.g., Antibody Engineering, Methods in Molecular Biology, vol. 248, edited by Benny K. C. Lo). A suitable human recipient framework for both the light and heavy chain variable domain may be identified by, for example, sequence or structural homology. Alternatively, fixed recipient frameworks may be used, e.g.; based on knowledge of structure, biophysical and biochemical properties. The recipient frameworks can be germline derived or derived from a mature antibody sequence. CDR regions from the donor antibody can be transferred by CDR grafting. The CDR grafted humanized antibody can be further optimized for e.g. affinity, functionality and biophysical properties by identification of critical framework positions where re-introduction (backmutation) of the amino acid residue from the donor antibody has beneficial impact on the properties of the humanized antibody. In addition to donor antibody derived backmutations the humanized antibody can be engineered by introduction of germline residues in the CDR or framework regions, elimination of immunogenic epitopes, site-directed mutagenesis, affinity maturation, etc.

Furthermore, humanized antibodies can comprise residues that are not found in the recipient antibody or in the donor antibody. These modifications are made to further refine antibody performance. In general, a humanized antibody will comprise at least one—typically two—variable domains, in which all or substantially all of the CDR regions correspond to those of a non-human immunoglobulin and in which all or substantially all of the FR residues are those of a human immunoglobulin sequence. The humanized antibody can, optionally, also comprise at least a portion of an immunoglobulin constant region (Fc), typically that of a human immunoglobulin. The term “humanized antibody derivative” refers to any modified form of the humanized antibody, such as a conjugate of the antibody and another agent or antibody.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg Nature Biotech. 23(9): 1117-1125 (2005)), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally occurring variants within a specific isotype group, which variants differ in a few amino acids (see, e.g., Jefferis et al., mAbs 1:1 (2009)). Anti-TREM-1 antibodies described herein can be of any allotype. In some embodiments, an anti-TREM-1 antibody of the present disclosure is of “IgG1,317” allotype, which comprises one or more amino acid substitutions selected from the group consisting of L234A, L235E, and G237A, per EU numbering, as compared to a wild-type IgG1 isotype (e.g., SEQ ID NO: 9). In other embodiments, an anti-TREM-1 is of “IgG1.1f” allotype, which comprises one or more amino acid substitutions selected from the group consisting of L234A, L235E, G237A, A3305, and P331S, per EU numbering, as compared to a wild-type IgG1 isotype (e.g., SEQ ID NO: 9). In further embodiments, an anti-TREM-1 antibody disclosed herein is of “IgG1-Aha” allotype, which comprises one or more amino acid substitutions selected from the group consisting of K214R, C226S, C229S, and P238S, per EU numbering, as compared to a wild-type IgG1 isotype (e.g., SEQ ID NO: 9). In some embodiments, an anti-TREM-1 antibody disclosed herein is of “IgG4-Aba” allotype, which comprises the CIE domain of a wild-type IgG4 isotype (e.g., SEQ ID NO: 10) and CH2 and CH3 domains of IgG1. In certain embodiments, the IgG4-Aba allotype antibody comprises one or more amino acid substitutions selected from the group consisting of S131C, K133R, G137E, G138S, Q196K, I199T, N203D, K214R, C2265, C2295, and P238S, per EU numbering, as compared to a wild-type IgG1 isotype (e.g., SEQ ID NO: 9).

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody that has been separated and/or recovered from (an)other component(s) in the environment in which it was produced and/or that has been purified from a mixture of components present in the environment in which it was produced.

An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor: BCR). Such effector functions generally require the Fc region to be combined with a binding domain (e.g., an antibody variable domain). In one embodiment, the anti-TREM-1 antibodies of the current disclosure comprise Fc regions that do not bind to one or more FcγRs and therefore, lack effector function (i.e., effectorless).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to F receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL).

In IgG, the Fc region comprises immunoglobulin domains CH2 and CH3 and the hinge between CH1 and CH2 domains. Although the definition of the boundaries of the Fe region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fe, including any allotypic variant, or a variant Fc (e.g., a non-naturally occurring Fe). Fc can also refer to this region in isolation or in the context of an Fe-comprising protein polypeptide such as a “binding protein comprising an Fc region,” also referred to as an “Fc fusion protein” (e.g., an antibody or immunoadhesion).

A “native sequence Fc region” or “native sequence Fe” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al., mAbs 1: 1 (2009)).

A “variant sequence Fc region” or “non-naturally occurring Fe” comprises a modification, typically to alter one or more of its functional properties, such as serum half-life, complement fixation, Fc-receptor binding, protein stability and/or antigen-dependent cellular cytotoxicity, or lack thereof, among others. In some embodiments, the anti-TREM-1 antibodies of the present disclosure can be chemically modified (e.g., one or more chemical moieties can be attached to the antibody) or be modified to alter its glycosylation, again to alter one or more functional properties of the antibody. In one embodiment, the anti-TREM-1 antibody is an IgG1 isotype and carries a modified Fe domain comprising one or more, and perhaps all of the following mutations that will result in decreased affinity to certain Fc receptors (L234A, L235E, and G237A) and in reduced C1q-mediated complement fixation (A330S and P331S), respectively (residue numbering according to the EU index).

The terms “hinge,” “hinge domain,” “hinge region,” and “antibody hinge region” refer to the domain of a heavy chain constant region that joins the CH1 domain to the CH2 domain and includes the upper, middle, and lower portions of the hinge (Roux et al., J Immunol 161:4083 (1998)). The hinge provides varying levels of flexibility between the binding and effector regions of an antibody and also provides sites for intermolecular disulfide bonding between the two heavy chain constant regions. As used herein, a hinge starts at Glu216 and ends at Gly237 of all IgG isotypes (Roux et al., J Immunol 161:4083 (1998)). The sequences of wildtype IgG1, IgG2, IgG3, and IgG4 hinges are known in the art (e.g., International PCT publication no. WO 2017/087678). In one embodiment, the hinge region of CHI of the anti-TREM-1 antibodies is modified such that the number of cysteine residues in the hinge region is altered, e.g., increased or decreased. This approach is described further for instance in U.S. Pat. No. 5,677,425.

The constant region may be modified to stabilize the antibody, e.g., to reduce the risk of a bivalent antibody separating into two monovalent VH-VL fragments. For example, in an IgG4 constant region, residue 5228 (residue numbering according to the EU index) may be mutated to a proline (P) residue to stabilize inter heavy chain disulphide bridge formation at the hinge (see, e.g., Angal et al., Mol Immunol. 30: 105-8(1995)). Antibodies or fragments thereof can also be defined in terms of their complementarity-determining regions (CDRs). The term “complementarity-determining region” or “hypervariable region”, when used herein, refers to the regions of an antibody in which amino acid residues involved in antigen binding are situated. The region of hypervariability or CDRs can be identified as the regions with the highest variability in amino acid alignments of antibody variable domains. Databases can be used for CDR identification such as the Kabat database, the CDRs e.g., being defined as comprising amino acid residues 24-34 (CDR1), 50-59 (CDR2) and 89-97 (CDR3) of the light-chain variable domain, and 31-35 (CDR1), 50-65 (CDR2) and 95-102 (CDR3) in the heavy-chain variable domain; (Kabat et al. 1991; Sequences of Proteins of Immunological Interest, Fifth Edition, U.S. Department of Health and Human Services, NIH Publication No. 91-3242) Alternatively CDRs can be defined as those residues from a “hypervariable loop” (residues 26-33 (L1), 50-52 (L2) and 91-96 (L3) in the light-chain variable domain and 26-32 (H1), 53-55 (H2) and 96-101 (H3) in the heavy-chain variable domain (Chothia and Lesk, J. Mol. Biol 196: 901-917 (1987)). Typically, the numbering of amino acid residues in this region is performed by the method described in Kabat et al., supra. Phrases such as “Kabat position”, “Kabat residue”, and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a framework (FR) or CDR of the variable domain. For example, a heavy chain variable domain may include amino acid insertions (residue 52a, 52b and 52c according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “epitope” or “antigenic determinant” refers to a site on an antigen (e.g., TREM-1) to which an immunoglobulin or antibody specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epi topes formed by tertiary folding are typically lost on treatment with denaturing solvents. An epitope typically includes at least 3, 4, 5, 6, 7, 8, 9, 10, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from TREM-1) are tested for reactivity with a given antibody (e.g., anti-TREM-1 antibody). Methods of determining spatial conformation of epitopes include techniques in the art and those described herein, for example, x-ray crystallography, antigen mutational analysis, 2-dimensional nuclear magnetic resonance and HDX-MS (see, e.g., Epitope Mapping Protocols in Methods in Molecular Biology, Vol. 66, G. E. Morris, Ed. (1996)).

The term “binds to the same epitope” with reference to two or more antibodies means that the antibodies bind to the same segment of amino acid residues, as determined by a given method. Techniques for determining whether antibodies bind to the “same epitope on TREM-1” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In certain embodiments, an antibody competes with, and inhibits binding of another antibody to a target by at least 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, N.Y., USA 1999. Two antibodies “cross-compete” if antibodies block each other both ways by at least 50%, i.e., regardless of whether one or the other antibody is contacted first with the antigen in the competition experiment.

As used herein, the terms “specific binding,” “selective binding,” “selectively binds,” and “specifically binds,” refer to antibody binding to an epitope on a predetermined antigen. Typically, the antibody (1) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e g, recombinant human TREM-1, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to human TREM-1” refers to an antibody that binds to soluble or cell bound human TREM-1 with a K_{D} of 10⁻⁷ M or less, such as approximately less than 10⁻⁸ M, 10⁻⁹ M or 10⁻¹ M or even lower. An antibody that “cross-reacts with cynomolgus TREM-1” refers to an antibody that binds to cynomolgus TREM-1 with a K_{D} of 10⁻⁷M or less, such as approximately less than 10⁻⁸ M, 10⁻⁹M or 10⁻¹⁰ M or even lower. In certain embodiments, such antibodies that do not cross-react with TREM-1 from a non-human species exhibit essentially undetectable binding against these proteins in standard binding assays.

As used herein, the term “anti-TREM-1 antibody” refers to an antibody (including fragments thereof) that specifically binds to human TREM-1. Unless stated otherwise, anti-TREM-1 antibodies disclosed herein are antagonistic antibodies inhibit or suppress the activity of TREM-1 (i.e., do not agonize upon binding) on cells, e.g., monocytes, macrophages, or neutrophils.

The term “binding specificity” herein refers to the interaction of a molecule such as an antibody, or fragment thereof, with a single exclusive antigen, or with a limited number of highly homologous antigens (or epitopes). In contrast, antibodies that are capable of specifically binding to TREM-1 are not capable of binding dissimilar molecules. Antibodies according to the invention may not be capable of binding Nkp44, the Natural killer cell p44-related protein.

The specificity of an interaction and the value of an equilibrium binding constant can be determined directly by well-known methods. Standard assays to evaluate the ability of ligands (such as antibodies) to bind their targets are known in the art and include, for example, ELISAs, Western blots, RIAs, and flow cytometry analysis. The binding kinetics and binding affinity of the antibody also can be assessed by standard assays known in the art, such as SPR.

Competitive binding assays for determining whether two antibodies compete or cross-compete for binding include: competition for binding to myeloid cells expressing TREM-1, e.g., by flow cytometry, such as described in the Examples. Other methods include: SPR (e.g, BIACORE®) solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al. J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the term “bin” is defined using a reference antibody. If a second antibody is unable to bind to an antigen at the same time as the reference antibody, the second antibody is said to belong to the same “bin” as the reference antibody. In this case, the reference and the second antibody competitively bind the same part of an antigen and are coined “competing antibodies”. If a second antibody is capable of binding to an antigen at the same time as the reference antibody, the second antibody is said to belong to a separate “bin”. In this case, the reference and the second antibody do not competitively bind the same part of an antigen and are coined “non-competing antibodies”.

Antibody “binning” does not provide direct information about the epitope. Competing antibodies, i.e., antibodies belonging to the same “bin” can have identical epitopes, overlapping epitopes, or even separate epitopes. The latter is the case if the reference antibody bound to its epitope on the antigen takes up the space required for the second antibody to contact its epitope on the antigen (“steric hindrance”). Non-competing antibodies generally have separate epitopes.

The term “binding affinity” herein refers to a measurement of the strength of a non-covalent interaction between two molecules, e.g. an antibody, or fragment thereof, and an antigen. The term “binding affinity” is used to describe monovalent interactions (intrinsic activity).

The binding affinity between two molecules, e.g. an antibody, or fragment thereof, and an antigen, through a monovalent interaction may be quantified by determination of the equilibrium dissociation constant (K_{D}). In turn, K_{D} can be determined by measurement of the kinetics of complex formation and dissociation, e.g. by the SPR method. The rate constants corresponding to the association and the dissociation of a, monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/kₐ. Following the above definition, binding affinities associated with different molecular interactions, such as comparison of the binding affinity of different antibodies for a given antigen, may be compared by comparison of the K_{D} values for the individual antibody/antigen complexes.

As used herein, the term “high affinity” for an IgG antibody refers to an antibody having a K_{D} of 10⁻⁸ M or less, 10⁻⁹ M or less, or 10⁻¹⁰ M or less for a target antigen. However, “high affinity” binding can vary for other antibody isotypes. For example, “high affinity” binding for an NM isotype refers to an antibody having a Kr) of 10⁻¹⁰ M or less, or 10⁻⁸M or less.

The term “EC₅₀” in the context of an in vitro or in vivo assay using an antibody or antigen binding fragment thereof, refers to the concentration of an antibody or an antigen-binding portion thereof that induces a response that is 50% of the maximal response, i.e., halfway between the maximal response and the baseline.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., al mine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, a predicted nonessential amino acid residue in an anti-TREM-1 antibody is replaced with another amino acid residue from the same side chain family. Methods of identiFying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Nod. Acad. Sci. USA. 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped. BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into whiCh additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein. “administering” refers to the physical introduction of a composition comprising a therapeutic agent to a subject, using any of the various methods and delivery systems known to those skilled in the art. Different routes of administration for the anti-TREM-1 antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the terms “inhibits” or “blocks” (e.g., referring to inhibition/blocking of binding of TREM-1 ligand to TREM-1 on cells) are used interchangeably and encompass both partial and complete inhibition/blocking. In some embodiments, the anti-TREM-1 antibody inhibits binding of TREM-1 ligand to TREM-1 by at least about 50%, for example, about 60%, 70%, 80%, 90%, 95%, 99%, or 100%, determined, e.g., as further described herein. In some embodiments, the anti-TREM-1 antibody inhibits binding of TREM-1 ligand to TREM-1 by no more than 50%, for example, by about 40%, 30%, 20%, 10%, 5% or 1%, determined, e.g., as further described herein.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, dog, cow, chickens, amphibians, reptiles, etc.

As used herein, the terms “ug” and “uM” are used interchangeably with “μg” and “μM,” respectively.

Various aspects described herein are described in further detail in the following subsections.

### II. Methods of the Present Disclosure

### Methods of Identifying a Subject Suitable for Treatment

Disclosed herein are methods of identifying a subject suffering from a disease or disorder suitable for a treatment with an anti-TREM-1 antibody (i.e., antagonistic anti-TREM-1 antibody). In some embodiments, a method disclosed herein comprises measuring an expression level of a TREM-1 associated gene in a sample of the subject. In some embodiments, a TREM-1 associated gene comprises one or more genes listed in Table 3 (below). In some embodiments, an expression level of a TREM-1 associated gene (e.g., disclosed herein) is increased upon binding of a natural TREM-1 ligand PGLYRP1) to TREM-1 but not upon binding of an agonistic anti-TRIM-1 antibody to TREM-1.

TABLE 3
TREM-1 Associated Genes

| Gene | Description |
|---|---|
| NAMPT | Nicotinamide phosphoribosyltransferase |
| DHRS9 | dehydrogenase/reductase |
| CDKN1A | cyclin dependent kinase inhibitor 1A |
| CD52 | CD52 molecule |
| MTMR11 | Myotubularin related protein 11 |
| EHD1 | EH domain containing 1 |
| SLC27A3 | Solute carrier family 27 member 3 |
| IL24 | Interleukin 24 |
| PIM2 | Pim-2 proto-oncogene, serine/threonine kinase |
| CHI3L1 | chitinase 3 like 1 |
| GALNT6 | Polypeptide N-acetylgalactosaminyltransferase 6 |
| ACOT7 | Acyl-CoA thioesterase 7 |
| CISH | cytokine inducible SH2 containing protein |
| FAM129A | family with sequence similarity 129 member A |
| PLK3 | polo like kinase 3 |
| MFSD12 | major facilitator superfamily domain containing 12 |
| STARD4 | StAR related lipid transfer domain containing 4 |
| CLEC12A | C-type lectin domain family 12 member A |
| CD55 | CD55 molecule (Cromer blood group) |
| IFNLRI | Interferon lambda receptor 1 |

In some embodiments, a subject suitable for a treatment with an anti-TREM-1 antibody exhibits an increased expression level of a TREM-1 associated gene compared to a reference (e.g., a subject not suffering from a disease or disorder, e.g., a healthy subject). In certain embodiments, an expression level of a TREM-1 associated gene (e.g., disclosed herein) in a subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject).

In some embodiments, an increased expression level of a TREM-1 associated gene disclosed herein is correlated with an increase in one or more other biological markers. In some embodiments, the one or more other biological markers comprise a baseline Mayo score, Grade 213 Lamina Propria Neutrophil Infiltration score, a fecal calprotectin level, or combinations thereof. Accordingly, in certain embodiments, a subject who is suitable for a treatment with an anti-TREM-1 antibody exhibits an increased baseline Mayo score, an increased Grade 2B Lamina Propria Neutrophil Infiltration score, and/or an increased fecal calprotectin level compared to a reference (e.g., a subject not suffering from a disease or disorder, e.g., a healthy subject). In some embodiments, a method of identifying a subject suitable for a treatment with an anti-TREM-1 antibody comprises determining a baseline Mayo score, Grade 2B Lamina Propria Neutrophil Infiltration score, and/or fecal calprotectin level in a sample of the subject.

In some embodiments, a baseline Mayo score of a subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject). In some embodiments, a baseline Mayo score of a subject is greater than about 6, about 7, about 8, about 9, about 10, about 11, or about 12.

In some embodiments, a Grade 213 Lamina Propria Neutrophil Infiltration score of a subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject). In some embodiments, a Grade 2B Lamina Propria Neutrophil Infiltration score of a subject is greater than about 0, about 0.1, about 0.2, or about 0.3.

In some embodiments, a fecal calprotectin level of subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject). In some embodiments, a fecal calprotectin level (μg/g) greater than about 1.5 log 10, about 2.0 log 10, about 2.5 log 10, about 3.0 log 10, or about 3.5 log 10.

In some embodiments, a method of identifying a subject suitable for an anti-TREM-1 antibody treatment disclosed herein, further comprises administering an effective dose of an anti-TREM-1 antibody to the subject.

### Methods of Determining Efficacy of Treatment

Also disclosed herein are methods of determining the efficacy of an anti-TREM-1 antibody (i.e., antagonistic anti-TREM-1 antibody) in treating a disease or disorder in a subject in need thereof. In some embodiments, a method disclosed herein comprises administering an anti-TREM-1 antibody to the subject and measuring an expression level of a TREM-1 associated gene in a sample of the subject. In some embodiments, a TREM-1 associated gene comprises one or more genes listed in Table 3 (above). In some embodiments, a TREM-1 associated gene (e.g., disclosed herein) is increased upon binding of a natural TREM-1 ligand (i.e., PGLYRP1) to TREM-1 but not upon binding of an agonistic anti-TREM-1 antibody to TREM-1.

In some embodiments, a subject exhibits a decreased expression level of a TREM-1 associated gene after the administration of the anti-TREM-1 antibody compared to a reference (e.g., corresponding value in the subject prior to the administration). In certain embodiments, an expression level of a TREM-1 associated gene in a subject is decreased after the administration by at least about 5%, at least about 10%, at least about 20%, at least about 30%, at least about 40%, at least about 50%, at least about 60%, at least about 70%, at least about 80%, at least about 90% or more compared to a reference (e.g., corresponding value in the subject prior to the administration). In some embodiments, a decreased expression level of a TREM-1 associated gene after the administration indicates that the anti-TREM-1 antibody is efficacious (e.g., reduces and/or prevents one or more symptoms associated with the disease or disorder) in the subject.

In some embodiments, a decreased expression level of a TREM-1 associated gene is correlated with a decrease in one or more other biological markers. In some embodiments, the one or more other biological markers comprise a baseline Mayo score, Grade 2B Lamina Propria Neutrophil Infiltration score, a fecal calprotectin level, or combinations thereof. Accordingly, in some embodiments, a method of determining the efficacy of an anti-TREM-1 antibody comprises administering an anti-TREM-1 antibody to the subject and determining a baseline Mayo score, Grade 2B Lamina Propria Neutrophil Infiltration score, and/or fecal calprotectin level in a sample of the subject. In some embodiments, an anti-TREM-1 antibody is efficacious (e.g., reduces and/or prevents one or more symptoms associated with the disease or disorder) where a baseline Mayo score. Grade 2B Lamina Propria Neutrophil Infiltration score, and/or fecal calprotectin level is decreased compared to a reference (e.g., corresponding value in the subject prior to the administration of the anti-TREM-1 antibody).

In some embodiments, a baseline Mayo score is decreased by at least about 5%, at least about 10%, at least about 20%, at least about 30%, at least about 40%, at least about 50%, at least about 60%, at least about 70%, at least about 80%, at least about 90% or more compared to a reference (e.g., corresponding value in the subject prior to the administration). In certain embodiments, a Grade 2B Lamina Propria Neutrophil Infiltration score is decreased by at least about 5%, at least about 10%, at least about 20%, at least about 30%, at least about 40%, at least about 50%, at least about 60%, at least about 70%, at least about 80%, at least about 90% or more compared to a reference (e.g., corresponding value in the subject prior to the administration). In some embodiments, a fecal calprotectin level is decreased by at least about 5%, at least about 10%, at least about 20%, at least about 30%, at least about 40%, at least about 50%, at least about 60%, at least about 70%, at least about 80%, at least about 90% or more compared to a reference (e.g., corresponding value in the subject prior to the administration).

In some embodiments, prior to the administration of an anti-TREM-1 antibody, a subject has a Baseline Mayo score of at least about 4, at least about 5, at least about 6, at least about 7, at least about 8, at least about 9, at least about 10, at least about 11, or at least about 12. In some embodiments, prior to the administration of an anti-TREM-1 antibody, a subject has a Grade 2B Lamina Propria Neutrophil Infiltration score of greater than about 0, greater than about 0.1, greater than about 0.2, or greater than about 0.3. In some embodiments, prior to the administration of an anti-TREM-1 antibody, a subject has a fecal calprotectin level that is greater than about 1.5 log 10, greater than about 2.0 log 10, greater than about 2.5 log 10, greater than about 3.0 log 1.0, or greater than about 3.5 log 10.

In some embodiments, measuring an expression level of a TREM-1 associated gene occurs at least 1 day, at least 2 days, at least 3 days, at least 4 days, at least 5 days, at least 6 days, at least 7 days, at least 2 weeks, at least 3 weeks, or at least 4 weeks or more after administering an anti-TREM-1 antibody to a subject. In some embodiments, determining a baseline Mayo score. Grade 2B Lamina Propria Neutrophil Infiltration score, and/or fecal calprotectin level occurs at least 1 day, at least 2 days, at least 3 days, at least 4 days, at least 5 days, at least 6 days, at least 7 days, at least 2 weeks, at least 3 weeks, or at least 4 weeks or more after administering an anti-TREM-1 antibody to a subject. In certain embodiments, measuring an expression level of a TREM-1 associated gene and/or determining a baseline Mayo score, Grade 2B Lamina Propria Neutrophil Infiltration score, and/or fecal calprotectin level occurs at multiple time points after the administration of the anti-TREM-1 antibody.

In some embodiments, a subject continues to receive the anti-TREM-1 antibody, wherein the anti-TREM-1 antibody treatment is determined to be efficacious in the subject. In some embodiments, a subject receives an adjusted dose of an anti-TREM-1 antibody, wherein the initial dose of the anti-TREM-1 antibody is determined not to be efficacious in the subject (e.g., an expression level of a TREM-1 associated gene. Mayo score, Grade 2B Lamina Propria Neutrophil Infiltration score, and/or fecal calprotectin level is not decreased in the subject after the administration).

### Methods of Identifying a Non-Responder to a Standard of Care Treatment

Present disclosure further provides methods of identifying a non-responder to a standard of care treatment for a disease or disorder. As used herein, the term “non-responder” refers to a subject who does not exhibit an improvement in one or more symptoms associated with the disease or disorder. As used herein, the term “standard of care treatment” refers to a treatment that is accepted by medical experts as a proper treatment for a certain type of disease and that is widely used by healthcare professionals. The term can be used interchangeable with any of the following terms: “best practice,” “standard medical care,” and “standard therapy.” In some embodiments, a disease or disorder comprises an inflammatory bowel disease (e.g., ulcerative colitis or Crohn's disease), and a standard of care treatment comprises drugs (e.g., anti-inflammatory agents, immunosuppressants, and antibiotics), nutrition supplements, and surgery. In certain embodiments, a standard of care treatment comprises an anti-TNF-α antibody, in some embodiments, an anti-TNF-α antibody comprises infliximab (REMICADE®), certolizumab pegol (CIMZIA®), etanercept (ENBREL®), adalimumab (HUMIRA®), golimumab (SIMPONI®), or combinations thereof. In other embodiments, a standard of care treatment comprises an oral corticosteroid. In some embodiments, a standard of care treatment comprises an anti-IP-10 antibody.

In some embodiments, a method of identifying a non-responder to a standard of care treatment comprises measuring an expression level of a TREM-1 associated gene in a sample of a subject who has previously received the standard of care treatment. In some embodiments, a TREM-1 associated gene comprises one or more genes listed in Table 3 (above). In some embodiments, a TREM-1 associated gene (e.g., disclosed herein) is increased upon binding of a natural TREM-1 ligand PGLYRP1) to TREM-1 but not upon binding of an agonistic anti-TREM-1 antibody to TREM-1.

In some embodiments, a subject is a non-responder if an expression level of a TREM-1 associated gene in the subject is increased compared to a reference a subject who is not suffering from a disease or disorder, e.g., a healthy subject). In certain embodiments, an expression level of a TREM-1 associated gene (e.g., disclosed herein) in a subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject).

In some embodiments, a subject is a non-responder if an expression level of a TREM-1 associated gene in the subject is not decreased compared to a reference (e.g., subject prior to the administration of the standard of care treatment). In some embodiments, an expression level of a TREM-1 associated gene in a subject is not decreased by at least about 5%, at least about 10%, at least about 20%, at least about 30%, at least about 40%, at least about 50%, at least about 60%, at least about 70%, at least about 80%, at least about 90% or more compared to a reference (e.g., subject prior to the administration of the standard of care treatment).

As described supra, in some embodiments, an expression level of a TREM-1 associated gene (e.g., disclosed herein) is correlated with one or more other biological markers, such as a baseline Mayo score, Grade 2B Lamina Propria Neutrophil Infiltration score, and/or fecal calprotectin level. Accordingly, in some embodiments, a method of identifying a non-responder to a standard of care treatment for a disease or disorder comprises determining a baseline Mayo score, Grade 2B Lamina Propria Neutrophil Infiltration score, and/or fecal calprotectin level in a sample of a subject who has previously received the standard of care treatment.

In some embodiments, a subject is a non-responder if a baseline Mayo score of the subject is increased compared to a reference (e.g., a subject who is not suffering from a disease or disorder, e.g., a healthy subject). In certain embodiments, a Baseline Mayo score of a non-responder is increased by at least 5%, at least 10%, at least 20%, at least 30%, at least 40%, at least 50%, at least 60%, at least 70%, at least 80%, at least 90% or more compared to a reference (e.g., a subject not suffering from the disease or disorder, e.g., a healthy subject). In certain embodiments, a non-responder to a standard of care treatment has a Baseline Mayo score of at least about 4, at least about 5, at least about 6, at least about 7, at least about 8, at least about 9, at least about 10, at least about 11, or at least about 12.

In some embodiments, a subject is a non-responder if a Grade 2B Lamina Propria Neutrophil Infiltration score of the subject is increased compared to a reference (e.g., a subject who is not suffering from a disease or disorder, e.g., a healthy subject). In some embodiments, a Grade 2B Lamina Propria Neutrophil Infiltration score of a non-responder is increased by at least 5%, at least 10%, at least 20%, at least 30%, at least 40%, at least 50%, at least 60%, at least 70%, at least 80%, at least 90% or more compared to the reference (e.g., a subject not suffering from the disease or disorder, e.g., a healthy subject). In certain embodiments, a Grade 2B Lamina Propria. Neutrophil Infiltration score is greater than about 0, greater than about 0.1, greater than about 0.2, or greater than about 0.3.

In some embodiments, a subject is a non-responder if a fecal calprotectin level of the subject is increased compared to a reference (e.g., a subject who is not suffering from a disease or disorder, e.g, a healthy subject). In some embodiments, a fecal calprotectin level of a non-responder disclosed herein is increased by at least 5%, at least 10%, at least 20%, at least 30% at least 40%, at least 50%, at least 60%, at least 70%, at least 80%, at least 90% or more compared to the reference (e.g., a subject not suffering from the disease or disorder, e.g., a healthy subject). In some embodiments, a fecal calprotectin level of a non-responder is greater than about 1.5 log 10, greater than about 2.0 log 10, greater than about 2.5 log 10, greater than about 3.0 log 10, or greater than about 3.5 log 10.

In some embodiments, a subject is a non-responder to a standard care of treatment if a Mayo score of the subject has not decreased after receiving the standard of care treatment. In certain embodiments, the subject's Mayo score has not decreased by at least about 5%, at least about 10%, at least about 20%, at least about 30%, at least about 40%, at least about 50%, at least about 60%, at least about 70%, at least about 80%, at least about 90% or more compared to a reference (e.g., subject prior to the administration of the standard of care treatment).

In some embodiments, a subject is a non-responder if a Grade 2B Lamina Propria Neutrophil Infiltration score of the subject has not decreased after receiving the standard of care treatment. In some embodiments, the subject's Grade 2B Lamina Propria Neutrophil Infiltration score has not decreased by at least about 5%, at least about 10%, at least about 20%, at least about 30%, at least about 40%, at least about 50%, at least about 60%, at least about 70%, at least about 80%, at least about 90% or more compared to a reference (e.g., subject prior to the administration of the standard of care treatment).

In some embodiments, a subject is a non-responder if a fecal calprotectin level in the subject has not decreased after receiving the standard of care treatment. In some embodiments, the subject's fecal calprotectin level has not decreased by at least about 5%, at least about 10%, at least about 20%, at least about 30%, at least about 40%, at least about 50%, at least about 60%, at least about 70%, at least about 80%, at least about 90% or more compared to a reference (e.g., subject prior to the administration of the standard of care treatment).

In some embodiments, a Baseline Mayo score of a non-responder subject prior to the administration of the standard of care treatment is at least about 4, at least about 5, at least about 6, at least about 7, at least about 8, at least about 9, at least about 10, at least about 11, or at least about 12. In some embodiments, a Grade 2B Lamina Propria Neutrophil Infiltration score of a non-responder subject prior to the administration of the standard of care treatment is greater than about 0, greater than about 0.1, greater than about 0.2, or greater than about 0.3. In some embodiments, a fecal calprotectin level of a non-responder subject prior to the administration of a standard of care treatment is greater than about 1.5 log 10, greater than about 2.0 log 10, greater than about 2.5 log 10, greater than about 3.0 log 10, or greater than about 3.5 log 10.

In some embodiments, measuring an expression level of a TREM-1 associated gene occurs at least 1 day, at least 2 days, at least 3 days, at least 4 days, at least 5 days, at least 6 days, at least 7 days, at least 2 weeks, at least 3 weeks, or at least 4 weeks or more after administering an anti-TREM-1 antibody to a subject. In some embodiments, determining a baseline Mayo score, Grade 2B Lamina Propria Neutrophil Infiltration score, and/or fecal calprotectin level occurs at least 1 day, at least 2 days, at least 3 days, at least 4 days, at least 5 days, at least 6 days, at least 7 days, at least 2 weeks, at least 3 weeks, or at least 4 weeks or more after administering an anti-TREM-1 antibody to a subject.

In some embodiments, a method disclosed herein further comprises administering an additional therapeutic agent to a subject who has been identified as a non-responder to the standard of care treatment. In certain embodiments, the additional therapeutic agent comprises an anti-TREM-1 antibody.

### Methods of Treating a Disease or Disorder

Disclosed herein are methods of treating a disease or disorder in a subject in need thereof, comprising administering an effective dose of an anti-TREM-1 antibody (i.e., antagonistic anti-TREM-1 antibody) to the subject, wherein the subject exhibits an increased expression level of a TREM-1 associated gene compared to a reference (e.g., a subject who is not suffering from a disease or disorder, e.g., a healthy subject). In some embodiments, a TREM-1 associated gene comprises one or more genes listed in Table 3 (above). In some embodiments, a TREM-1 associated gene (e.g., disclosed herein) is increased upon binding of a natural TREM-1 ligand (i.e., PGLYRP1) to TREM-1 but not upon binding of an agonistic anti-TREM-1 antibody to TREM-1.

In some embodiments, an expression level of a TREM-1 associated gene in a subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject).

In some embodiments, administering an anti-TREM-1 antibody to the subject decreases an expression level of a TREM-1 associated gene compared to a reference (e.g., corresponding value in the subject prior to the administration). In certain embodiments, an expression level of a MEM-1 associated gene is decreased by at least about 5%, at least about 10%, at least about 20%, at least about 30%, at least about 40%, at least about 50%, at least about 60%, at least about 70%, at least about 80%, at least about 90% or more after the administration.

In some embodiments, prior to the administration of the anti-TREM-1 antibody, a subject exhibits an increased baseline Mayo score, an increased Grade 2B Lamina Propria Neutrophil Infiltration score, and/or an increased fecal calprotectin level compared to a reference (e.g., a subject not suffering from a disease or disorder, e.g., a healthy subject).

In some embodiments, a baseline Mayo score of a subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject). In some embodiments, a baseline Mayo score of a subject is greater than about 6, greater than about 7, greater than about 8, greater than about 9, greater than about 10, greater than about 11, or greater than about 12 prior to the administration of the anti-TREM-1 antibody.

In some embodiments, a Grade 213 Lamina Propria Neutrophil Infiltration score of a subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject). In some embodiments, a Grade 2B Lamina Propria Neutrophil Infiltration score of a subject is greater than about 0, greater than about 0.1, greater than about 0.2, or greater than about 0.3 prior to the administration of the anti-TREM-1 antibody.

In some embodiments, a fecal calprotectin level of a subject is increased by at least about 5%, at least about 10%, at least about 20%, at least about 30%, at least about 40%, at least about 50%, at least about 60%, at least about 70%, at least about 80%, at least about 90% or more compared to a reference (e.g., a subject not suffering from a disease or disorder, e.g., a healthy subject). In some embodiments, a fecal calprotectin level (μg/g) is greater than about 1.5 log 10, greater than about 2.0 log 10, greater than about 2.5 log 10, greater than about 3.0 log 10, or greater than about 3.5 log 10 prior to the administration.

In some embodiments, administering an anti-TREM-1 antibody reduces a baseline Mayo score, Grade 2B Lamina Propria Neutrophil infiltration score, and/or fecal calprotectin level in the subject compared to a reference (e.g., corresponding value in the subject prior to the administration). In some embodiments, a baseline Mayo score is decreased by at least about 5%, at least about 10%, at least about 20%, at least about 30%, at least about 40%, at least about 50%, at least about 60%, at least about 70%, at least about 80%, at least about 90% or more after the administration. In certain embodiments, a Grade 2B Lamina Propria Neutrophil Infiltration score is decreased by at least about 5%, at least about 10%, at least about 20%, at least about 30%, at least about 40%, at least about 50%, at least about 60%, at least about 70%, at least about 80%, at least about 90% or more after the administration. In some embodiments, a fecal calprotectin level is decreased by at least about 5%, at least about 10%, at least about 20%, at least about 30%, at least about 40%, at least about 50%, at least about 60%, at least about 70%, at least about 80%, at least about 90% or more after the administration.

In some embodiments, a method of treating a disease or disorder in a subject in need thereof, further comprises measuring an expression level of a TREM-1 associated gene and/or determining a baseline Mayo score, Grade 2B Lamina Propria Neutrophil Infiltration score, and/or fecal calprotectin level prior to administering an anti-TREM-1 antibody to the subject.

In some embodiments, a method of treating a disease or disorder disclosed herein, comprises measuring an expression level of a TREM-1 associated gene and/or determining a baseline Mayo score. Grade 2B Lamina Propria Neutrophil Infiltration score, and/or fecal calprotectin level after administering an anti-TREM-1 antibody to the subject. In certain embodiments, measuring an expression level of a TREM-1 associated gene and/or determining a baseline Mayo score, Grade 2B Lamina Propria Neutrophil Infiltration score, and/or fecal calprotectin level occurs at least 1 day, at least 2 days, at least 3 days, at least 4 days, at least 5 days, at least 6 days, at least 7 days, at least 2 weeks, at least 3 weeks, or at least 4 weeks or more after administering the anti-TREM-1 antibody.

Methods and compositions disclosed herein can be used for e.g., to treat) a wide variety of diseases or disorders, wherein the diseases or disorders are associated with an increase in TREM-1 activity. In some embodiments, a disease or disorder is associated with increased degranulation, reactive oxygen species formation, and/or release of pro-inflammatory cytokines by neutrophils. In certain embodiments, a disease or disorder is associated with activation of monocytes and/or increased production of inflammatory cytokines and chemokines by monocytes. In some embodiments, a disease or disorder is associated with hypoxia. In some embodiments, a disease or disorder is associated with an increase in cell surface TREM-1 protein expression and/or an increase in level of soluble TREM-1 protein.

Non-limiting examples of such diseases include inflammatory bowel disease (IBD), Crohn's disease (CD), ulcerative colitis (UC), irritable bowel syndrome, rheumatoid arthritis (RA), psoriasis, psoriatic arthritis, systemic lupus erythematosus (SLE), lupus nephritis, type I diabetes, Grave's disease, multiple sclerosis (MS), autoimmune myocarditis, Kawasaki disease, coronary artery disease, chronic obstructive pulmonary disease, interstitial lung disease, autoimmune thyroiditis, scleroderma, systemic sclerosis, osteoarthritis, atopic dermatitis, vitiligo, graft versus host disease, Sjogrens's syndrome, autoimmune nephritis, Goodpasture's syndrome, chronic inflammatory demyelinating polyneuropathy, allergy, asthma and other autoimmune diseases that are a result of either acute or chronic inflammation. In some embodiments, a disease or disorder is an inflammatory bowel disease. In certain embodiments, the inflammatory bowel disease comprises Crohn's disease and ulcerative colitis.

In some embodiments, a sample of a subject in which an expression level of a TREM-1 associated gene is measured comprises a tissue, blood, serum, plasma, saliva, urine, or combinations thereof. In some embodiments, a sample of a subject in which a baseline Mayo score, Grade 2B Lamina Propria Neutrophil Infiltration score, or a calprotectin level is determined comprises a tissue, blood, serum, plasma, saliva, urine, or combinations thereof.

### III. Anti-TREM-1 Antibodies

Certain aspects of the present disclosure comprise administering to a subject in need thereof a therapeutically effective amount of an anti-TREM-1 antibody (i.e., an antagonistic anti-TREM-1 antibody). Anti-TREM-1 antibodies (or VH/VL domains derived therefrom) suitable for use in the present disclosure can be generated using methods known in the art. Alternatively, art recognized anti-TREM-1 antibodies can be used. See, e.g., WO 2016/009086 and WO 2017/152102; each of which is herein incorporated by reference in its entirety.

Anti-TREM-1 antibodies useful in the methods disclosed herein (e.g., fully human monoclonal antibodies) are characterized by particular functional features or properties, which are provided throughout the detailed description. In some embodiments, an anti-TREM-1 antibody that can be used with the present methods exhibit one or more of the following properties:

(a) binds to soluble and/or membrane bound human TREM-1 (e.g., at the site on the extracellular domain to which the TREM-1 ligand (e.g., PGLYRP1) binds);
(b) cross-reacts with TREM-1 from one or more non-human primates cynomolgus TREM-1);
(c) blocks or inhibits the binding of PGLYRP1 to TREM-1;
(d) blocks or inhibits the production of inflammatory cytokines IL-6, TNF-α, IL-8, IL-1β, IL-12, and combinations thereof) by cells (e.g., macrophages, dendritic cells, neutrophils) upon activation;
(e) does not induce the release of proinflammatory cytokines by myeloid cells (e.g., dendritic cells);
(f) does not bind to one or more FcγRs;
(g) has a viscosity profile of less than about 5 cP at a concentration of 80 mg/mL or less than about 10 cP at a concentration of 130 mg/mL, and/or
(h) reduces or prevents the onset of inflammatory cytokine storm after administration to a subject.

In some embodiments, anti-TREM-1 antibodies described herein bind to human TREM-1 with high affinity, e.g., as determined by BIACORE™ (e.g., as described in the Examples), with a Ku of 10⁻⁷M or less, 10⁻⁸M or less, 10⁻⁹M (1 nM) or less, 10⁻¹⁰ M or less, 10⁻¹¹ M or less, 10⁻¹² M or less, 10⁻¹²M to 10⁻⁷M, 10⁻¹¹M to 10⁻⁷M, 10⁻¹⁰ M to 10⁻⁷ M, or 10⁻⁹ M, or 10⁻⁹M to 10⁻⁷ M. In some embodiments, anti-TREM-1 antibodies described herein bind to cyno TREM-1, e.g., as determined by BIACORE™ (e.g., as described in the Examples), with a K_{D} of 10⁻⁷M or less, 10⁻⁸ M or less, 10⁻⁹ M or less, 10⁻¹⁰ M or less, 10⁻¹¹ M or less, 10⁻¹²M or less, 10⁻¹² \1 to 10⁻⁷M, 10⁻¹¹M to 10⁻⁷M, 10⁻¹⁰ M to 10⁻⁷M, or 10⁻⁹ M to 10⁻⁷M.

In some embodiments, an anti-TREM-1 antibody cross-competes with mAb 0170 and/or mAb 0318 for binding to human TREM-1. In certain embodiments, an anti-TREM-1 antibody also cross-competes with mAb 0170 and/or mAb 0318 for binding to cynomolgus TREM-1. In other words, an anti-TREM-1 antibody that can be used with the present methods belong to the same “bin” as mAb 0170 and/or mAb 0318 in certain embodiments.

The mAb 0170 antibody has a heavy chain variable region (VH) and a light chain variable region (VL), wherein the VH comprises amino acids 1 to 121 of SEQ ID NO: 13 and wherein the VL comprises amino acids 1 to 111 of SEQ ID NO: 14. The mAb 0170 antibody also has a heavy chain CDR1, CDR2, and CDR3 and a light chain CDR1, CDR2, and CDR3, wherein (a) the heavy chain CDR1 comprises amino acids 31 to 35 of SEQ ID NO: 13; (b) the heavy chain CDR2 comprises amino acids 50 to 68 of SEQ ID NO: 13; (c) the heavy chain CDR3 comprises amino acids 101 to 110 of SEQ ID NO: 13; (d) the light chain CDR1 comprises amino acids 24 to 38 of SEQ ID NO: 14; (e) the light chain CDR2 comprises amino acids 54 to 60 of SEQ ID NO: 14; and (f) the light chain CDR3 comprises amino acids 93 to 101 of SEQ ID NO: 14. See WO 2016/009086, which is herein incorporated by reference in its entirety.

Accordingly, in some embodiments, an anti-TREM-1 antibody useful for the present disclosure comprises a VH and a VL, wherein the VH comprises the amino acid sequence set forth in SEQ ID NO: 15 (i.e., amino acids 1 to 121 of SEQ ID NO: 13) and wherein the VL comprises the amino acid sequence set forth in SEQ ID NO: 16 (i.e., amino acids 1 to 111 of SEQ ID NO: 14). In some embodiments, the VH of an anti-TREM-1 antibody comprises a CDR1 sequence as set forth in SEQ ID NO: 17 (TYAMH), a CDR2 sequence as set forth in SEQ ID NO: 18 (RIRTKSSNYATYYAASVKG), and a CDR3 sequence as set forth in SEQ ID NO: 19 (DMGIRRQFAY). In some embodiments, the VL of an anti-TREM-1 antibody comprises a CDR1 sequence as set forth in SEQ ID NO: 20 (RASESVDTFDYSFLH), a CDR2 sequence as set forth in SEQ ID NO: 21 (RASNLES), and a CDR3 sequence as set forth in SEQ ID NO: 22 (QQSNEDPYT).

The mAb 0318 antibody has a heavy chain variable region (VH) comprising SEQ ID NO: 15 and a light chain variable region (VL) comprising SEQ ID NO: 23. See International Publ. No. 2016/009086. The mAb 0318 also has a heavy chain CDR1, CDR2, and CDR3, which correspond to amino acids 31-35, 50-68, and 101-110 of SEQ ID NO: 15, respectively. The light chain CDR1, CDR2, and CDR3 of the mAb 0318 antibody correspond to amino acids 24-38, 54-60, and 93-101 of SEQ ID NO: 23.

Accordingly, in some embodiments, an anti-TREM-1 antibody comprises a VH and VL of SEQ ID NOs: 15 and 23, respectively. In some embodiments, the VH of the anti-TREM-1 antibody comprises a CDR1 sequence of amino acids 31-35 (TYAMH) of SEQ ID NO: 15, wherein one of the amino acids can be substituted by a different amino acid. In certain embodiments, the VH of the anti-TREM-1 antibody comprises a CDR2 sequence of amino acids 50-68 (RIRTKSSNYATYYAASVKG) of SEQ ID NO: 15, wherein one, two, or three of the amino acids can be substituted by a different amino acid. In some embodiments, the VH of the anti-TREM-1 antibody comprises a CDR3 sequence of amino acids 101-110 (DMGIRRQFAY) of SEQ ID NO: 15, wherein one, two, or three of the amino acids can be substituted by a different amino acid.

In some embodiments, the VL of the anti-TREM-1 antibody comprises a CDR1 sequence of amino acids 24-38 (RASQSVDTFDYSFLH) of SEQ ID NO: 23, wherein one, two, or three of the amino acids can be substituted by a different amino acid. In other embodiments, the VL of the anti-TREM-1 antibody comprises a CDR2 sequence of amino acids 54-60 (RASNLES) of SEQ ID NO: 23, wherein one or two of the amino acids can be substituted with a different amino acid. In some embodiments, the VL of the anti-MEM-1 antibody comprises a CDR3 sequence of amino acids 93-101 (QQSNQDPYT) of SEQ ID NO: 23, wherein one or two of the amino acids can be substituted with a different amino acid.

Methionine residues in CDRs of antibodies can be oxidized, resulting in potential chemical degradation and consequent reduction in potency of the antibody. Accordingly, the anti-TREM-1 antibodies disclosed herein can have one or more methionine residues in the heavy and/or light chain CDRs replaced with amino acid residues which do not undergo oxidative degradation. In some embodiments, the methionine residues within the heavy chain CDR1 and CDR3 are replaced with amino acid residues that do not undergo oxidative degradation (e.g., glutamine or leucine). Accordingly, in some embodiments, the VH of the anti-TREM-1 antibody comprises a CDR3 sequence of amino acids 101-110 (DQGIRRQFAY) of SEQ ID NO: 26 or amino acids 101-110 (DLGIRRQFAY) of SEQ ID NO: 27. In other embodiments, the VH of the anti-TREM-1 antibody comprises a CDR1 sequence of amino acids 31-35 (TYAQH) of SEQ ID NO: 28 or amino acids 31-35 (TYALH) of SEQ ID NO: 29. Similarly, in some embodiments, deamidation sites can be removed from the anti-TRIM-1 antibodies, particularly in the CDRs.

In some embodiments, the VH and VL of the anti-TREM-1 antibody comprises a VH and VL sequences of anti-TREM-1 antibodies disclosed in International Publ. No. WO 2017/152102 A2, which is herein incorporated by reference in its entirety. In some embodiments, the VL of the anti-TREM-1 antibody comprises a CDR1 sequence selected from the group consisting of SEQ D NOs: 9-27 from WO 2017/152102, a CDR2 sequence selected from the group consisting of SEQ ID NOs: 28-40 from WO 2017/152102, and/or a CDR3 sequence selected from the group consisting of SEQ ID NOs: 41-119 from WO 2017/152102. In one embodiment, the VH of the anti-TREM-1 antibody comprises a CDR1 sequence selected from the group consisting of SEQ ID NOs: 120-143 from WO 2017/152102, a CDR2 sequence selected from the group consisting of SEQ ID NOs: 144-172 from WO 2017/152102, and/or a CDR3 sequence selected from the group consisting of SEQ ID NOs: 173-247 from WO 2017/152102.

In some embodiments, the anti-TREM-1 antibodies of the present disclosure comprise CDR and/or variable region sequences that have at least 80% identity (e.g., at least 85%, at least 95%, at least 95%, or at least 99% identity) to the CDR and/or variable region sequences of the mAb 0318 antibody.

In some embodiments, the anti-TREM-1 antibodies of the present disclosure comprise a heavy chain variable region (VH) selected from the group consisting of SEQ ID NOs: 396-475 from WO 2017/152102 and/or a light chain variable region (VL) selected from the group consisting of SEQ ID NOs: 316-395 from WO 2017/152102.

In some embodiments, the anti-TREM-1 antibodies comprise a heavy chain (HC) and a light chain (LC), wherein the HC comprises SEQ ID NO: 50, SEQ ID NO: 51, SEQ ID NO: 52, or SEQ ID NO: 53. In some embodiments, the LC comprises SEQ ID NO: 54.

In some embodiments, the anti-TREM-1 antibody comprises a heavy chain and a light chain, wherein the heavy chain and light chain comprises amino acid sequences as shown in Table 7. In some embodiments, the anti-TREM-1 antibody comprises a heavy chain and a light chain, wherein the heavy chain comprises the amino acid sequence set forth as SEQ ID NO: 30 and the light chain comprises the amino acid sequence set forth as SEQ ID NO: 34. In some embodiments, the anti-TREM-1 antibody comprises a heavy chain and a light chain, wherein the heavy chain comprises the amino acid sequence set forth as SEQ ID NO: 31 and the light chain comprises the amino acid sequence set forth as SEQ ID NO: 34. In some embodiments, the anti-TREM-1 antibody comprises a heavy chain and a light chain, wherein the heavy chain comprises the amino acid sequence set forth as SEQ ID NO: 32 and the light chain comprises the amino acid sequence set forth as SEQ ID NO: 34. In some embodiments, the anti-TREM-1 antibody comprises a heavy chain and a light chain, wherein the heavy chain comprises the amino acid sequence set forth as SEQ ID NO: 33 and the light chain comprises the amino acid sequence set forth as SEQ ID NO: 34.

Heavy and light chains comprising an amino acid sequence that is at least 99%, 98%, 97%, 96%, 95%, 90%, 85%, or 80% identical to any of the heavy or light chains set forth herein, e.g., SEQ ID NOs: 30 to 34 can be used for forming the anti-TREM-1 antibodies having the desired characteristics, e.g., those further described herein.

In some embodiments, the anti-TREM-1 antibody of the present disclosure comprises a heavy chain and a light chain, wherein the heavy chain comprises an amino acid sequence that is at least 80%, at least 85%, at least 90%, at least 95%, at least 96%, at least 97%, at least 98%, at least 99%, or 100% identical to the amino acid sequence set forth as SEQ ID NO: 30, 31, 32, or 33 and wherein the light chain comprises an amino acid sequence that is at least 80%, at least 85%, at least 90%, at least 95%, at least 96%, at least 97%, at least 98%, at least 99%, or 100% identical to the amino acid sequence set forth as SEQ ID NO: 34.

In some embodiments, an anti-TREM-1 antibody that can be used with the present methods is epitope-steered. As used herein, the term “epitope-steered” refers to anti-TREM-1 antibodies that are selected to bind to epitopes other than D38 to L45, E46 to Q56, and/or Y90 to L96 of human TRIM-1 (SEQ ID NO: 1). In some embodiments, the epitope-steered anti-TREM-1 antibodies bind to one or more epitope selected from the group consisting of (1) ²⁷EKYELKEGQTL³⁷ (SEQ ID NO: 50), (2) ⁸⁸EDYHDHGLLRVRM¹⁰⁰ (SEQ ID NO: 51), (3) ¹²⁰KEPHMLFDR¹²⁸ (SEQ ID NO: 52), and any combination thereof of human TREM-1 (e.g., Isoform 1, SEQ ID NO: 1).

Epitope-steered anti-TREM-1 antibodies described herein can be produced by any method known in the art, such as those described in the Examples. In some embodiments, the epitope-steered anti-TREM-1 antibodies can be generated by immunizing an animal (e.g., mice) with a human TREM-1 polypeptide comprising mutations at one of above epitopes (e.g., amino acids residues 38-48 of SEQ ID NO: 1). Upon immunization, the antibodies generated can be further characterized for binding to human TREM-1. In some embodiments, synthetic peptides that comprise the epitope of interest can be synthesized and used to immunize an animal (e.g., mice). In some embodiments, alternative scaffolds (e.g., tenth human fibronectin type three domain, 10Fn3; or α3D, a highly thermostable three-helix bundle protein) that comprise the epitope of interest can be used.

In some embodiments, an anti-TREM-1 antibody (i.e., epitope-steered) does not cross-compete with mAb 0170 and/or mAb 0318 for binding to TREM-1 (e.g., human or cynomolgus). In other words, in certain embodiments, an anti-TREM-1 antibody disclosed herein belongs to a different “bin” as mAb 0170 and/or mAb 0318.

In some embodiments, an epitope-steered anti-TREM-1 antibody comprises a VII and a VL, wherein:

(a) the VH and VL comprises amino acid sequences set forth as SEQ ID NOs: 53 and 54, respectively;
(b) the VII and VL comprises amino acid sequences set forth as SEQ ID NOs: 55 and 56, respectively;
(c) the VH and VL comprises amino acid sequences set forth as SEQ ID NOs: 55 and 57, respectively;
(d) the VH and VL comprises amino acid sequences set forth as SEQ ID NOs: 58 and 59, respectively;
(e) the VH and VL comprises amino acid sequences set forth as SEQ ID NOs: 60 and 56, respectively.
(f) the VH and VL, comprises amino acid sequences set forth as SEQ ID NOs: 153 and 154, respectively; or
(g) the VII and VL comprises amino acid sequences set forth as SEQ ID NOs: 153 and 155, respectively.

In some embodiments, an epitope-steered anti-TREM-1 antibody disclosed herein comprises CDRs of a heavy chain variable region selected from the group consisting of SEQ ID NOs: 53, 55, 58, 60, and 153. In some embodiments, an epitope-steered anti-TREM-1 antibody disclosed herein comprises CDRs of a light chain variable region selected from the group consisting of SEQ ID NOs: 54, 56, 57, 59, 154, and 155.

In some embodiments, an epitope-steered anti-TREM-1 antibodies that can be used with the present methods comprises a heavy chain variable region (VH) CDR1, CDR2, and CDR3, and a light chain variable region (VL) CDR1, CDR2, and CDR3, wherein:

(a) the VH CDR1 comprises the amino acid sequence set forth as SEQ ID NO: 61, the VH CDR2 comprises the amino acid sequence set forth as SEQ ID NO: 62, and the VH CDR3 comprises the amino acid sequence set forth as SEQ ID NO: 63, the VL CDR1 comprises the amino acid sequence set forth as SEQ ID NO: 64, the VL CDR2 comprises the amino acid sequence set forth as SEQ ID NO: 65, and the VL CDR3 comprises the amino acid sequence set forth as SEQ ID NO: 66;
(b) the VH CDR1 comprises the amino acid sequence set forth as SEQ ID NO: 67, the VH CDR2 comprises the amino acid sequence set forth as SEQ ID NO: 68, and the VH CDR3 comprises the amino acid sequence set forth as SEQ ID NO: 69, the VL CDR1 comprises the amino acid sequence set forth as SEQ ID NO: 70, the VL CDR2 comprises the amino acid sequence set forth as SEQ ID NO: 71, and the VL CDR3 comprises the amino acid sequence set forth as SEQ ID NO: 72;
(c) the VH CDR1 comprises the amino acid sequence set forth as SEQ ID NO: 67, the VH CDR2 comprises the amino acid sequence set forth as SEQ ID NO: 68, and the VH CDR3 comprises the amino acid sequence set forth as SEQ ID NO: 69, the VL CDR1 comprises the amino acid sequence set forth as SEQ ID NO: 64, the VL CDR2 comprises the amino acid sequence set forth as SEQ ID NO: 65, and the VL CDR3 comprises the amino acid sequence set forth as SEQ ID NO: 38;
(d) the VU CDR1 comprises the amino acid sequence set forth as SEQ ID NO: 74, the VH CDR2 comprises the amino acid sequence set forth as SEQ ID NO: 75, and the VH CDR3 comprises the amino acid sequence set forth as SEQ ID NO: 76, the VL CDR1 comprises the amino acid sequence set forth as SEQ ID NO: 70, the VL CDR2 comprises the amino acid sequence set forth as SEQ ID NO: 77, and the VL CDR3 comprises the amino acid sequence set forth as SEQ ID NO: 78;
(e) the VH CDR1 comprises the amino acid sequence set forth as SEQ ID NO: 79, the VH CDR2 comprises the amino acid sequence set forth as SEQ ID NO: 80, and the VH CDR3 comprises the amino acid sequence set forth as SEQ ID NO: 81, the VL CDR1 comprises the amino acid sequence set forth as SEQ ID NO: 70, the VL CDR2 comprises the amino acid sequence set forth as SEQ ID NO: 71, and the VL CDR3 comprises the amino acid sequence set forth as SEQ ID NO: 72;
(f) the VH CDR1 comprises the amino acid sequence set forth as SEQ ID NO: 159, the VH CDR2 comprises the amino acid sequence set forth as SEQ ID NO: 160, and the VH CDR3 comprises the amino acid sequence set forth as SEQ ID NO: 161, the VL CDR1 comprises the amino acid sequence set forth as SEQ ID NO: 70, the VL CDR2 comprises the amino acid sequence set forth as SEQ ID NO: 71, and the VL CDR3 comprises the amino acid sequence set forth as SEQ ID NO: 162; or
(g) the VH CDR1 comprises the amino acid sequence set forth as SEQ ID NO: 159, the VU CDR2 comprises the amino acid sequence set forth as SEQ ID NO: 160, and the VU CDR3 comprises the amino acid sequence set forth as SEQ ID NO: 161, the VL CDR1 comprises the amino acid sequence set forth as SEQ ID NO: 70, the VL CDR2 comprises the amino acid sequence set forth as SEQ ID NO: 71, and the VL CDR3 comprises the amino acid sequence set forth as SEQ ID NO: 133.

In some embodiments, an epitope-steered anti-TREM-1 antibody that can be used with the present methods comprises heavy chain variable region (VH) CDR1, CDR2, and CDR3 and a light chain variable region (VL) CDR1, CDR2, and CDR3, wherein one or more of the CDRs comprise one or more amino acid mutations (e.g., substitution or deletion) relative to an anti-TREM-1 antibody disclosed herein. Accordingly, in certain embodiments, an epitope-steered anti-TREM-1 antibody comprises a VH CDR1 comprising X1, X2, X3, X4, and X5, wherein X1 is S or N; X2 is S, Y, or E; X3 is Y G, or A; X4 is W, M or I; and X5 is S, T, H, or N. In some embodiments, an epitope-steered anti-TREM-1 antibody comprises a VH CDR2 comprising X1, X2, X3, X4, X5, X6, X7, X8, X9, X10, X11, X12, X13, X14, X15, X16, and X17, wherein X1 is Y V, or G; X2 is T or I; X3 is W, I, or none; X4 is H, Y, or P; X5 is Y, D, or I; X6 is S, G, or F; X7 is G, S, or D; X8 is I; Y, N, or T; X9 is S, T, or K; X10 is N or Y; X11 is Y or G; X12 is N or A; X13 is P, D, or Q; X14 is S or K; X15 is L, V, or F; X16 is K or Q; and X17 is S or G. In some embodiments, an epitope-steered anti-TREM-1 antibody comprises a VH CDR3 comprising X1, X2, X3, X4, X5. X6, X7, X8, X9, X10, X11, X12. G, X13, X14, X15, X16, X17, X18, D, and X19, wherein X1 is E, D, M, T, or none; X2 is G, V, or Y; X3 is Y, R, or none; X4 is D, H, G, or none; X5 is I, Y, or none; X6 is L, V, or none; X7 is T, G, N, or none; X8 is G, 5, Y, or none; X9 is Y, V, T, F, or H; X10 is E, L, 5, or Y; X11 is Y, W, F, or H; X12 is Y or F; X13 is E or none; X14 is L or none; X15 is 1, or none; X16 is P or none; X17 is L or none; X18 is M or L; and X19 is V or Y. In certain embodiments, an epitope-steered anti-TREM-1 antibody comprises a VL CDR1 comprising R, A, 5, Q, X1, X2, X3, S, S, X4, L, and A, wherein X1 is S or G; X2 is V or I; X3 is S or none; and X4 is Y or A. In some embodiments, an epitope-steered anti-TREM-1 antibody comprises a VL CDR2 comprising X1, A, S, S, X2, X3, and X4, wherein X1 is G, D or A; X2 is R or L; X3 is A, E, or Q; and X4 is T or S. In certain embodiments, an epitope-steered anti-TREM-1 antibody comprises a VL CDR3 comprising Q, Q, XL X2, S, X3, P, X4, and T, wherein X1 is Y or F; X2 is (3 or N; X3 is S or Y; and X4 is L, Y, I, or none.

In some embodiments, an anti-TREM-1 antibody useful for the present disclosure comprises a heavy chain variable region (VH) and a light chain variable region (VL), wherein

(a) the VH comprises the amino acid sequence set forth in SEQ ID NO: 82 and the VL comprises the amino acid sequence set forth in SEQ ID NO: 83;
(b) the VH comprises the amino acid sequence set forth in SEQ ID NO: 84 and the VL comprises the amino acid sequence set forth in SEQ ID NO: 85;
(c) the VH comprises the amino acid sequence set forth in SEQ ID NO: 86 and the VL comprises the amino acid sequence set forth in SEQ ID NO: 87;
(d) the VH comprises the amino acid sequence set forth in SEQ ID NO: 88 and the VL comprises the amino acid sequence set forth in SEQ ID NO: 89;
(e) the VH comprises the amino acid sequence set forth in SEQ ID NO: 88 and the VL comprises the amino acid sequence set forth in SEQ ID NO: 90;
(f) the VH comprises the amino acid sequence set forth in SEQ ID NO: 88 and the NIL comprises the amino acid sequence set forth in SEQ ID NO: 83;
(g) the VH comprises the amino acid sequence set forth in SEQ ID NO: 91 and the VL comprises the amino acid sequence set forth in SEQ ID NO: 90;
(h) the NTH comprises the amino acid sequence set forth in SEQ ID NO: 88 and the VL comprises the amino acid sequence set forth in SEQ ID NO: 92;
(i) the VH comprises the amino acid sequence set forth in SEQ ID NO: 93 and the VL comprises the amino acid sequence set forth in SEQ ID NO: 94;
(j) the VH comprises the amino acid sequence set forth in SEQ ID NO: 95 and the VL comprises the amino acid sequence set forth in SEQ ID NO: 96;
(k) the VH comprises the amino acid sequence set forth in SEQ ID NO: 97 and the VL comprises the amino acid sequence set forth in SEQ ID NO: 83;
(l) the VH comprises the amino acid sequence set forth in SEQ ID NO: 97 and the VL comprises the amino acid sequence set firth in SEQ ID NO: 98;
(m) the VH comprises the amino acid sequence set forth in SEQ ID NO: 97 and the NIL comprises the amino acid sequence set forth in SEQ ID NO: 99;
(n) the VH comprises the amino acid sequence set forth in SEQ ID NO: 97 and the VL comprises the amino acid sequence set forth in SEQ ID NO: 100;
(o) the VH comprises the amino acid sequence set forth in SEQ ID NO: 97 and the VL comprises the amino acid sequence set forth in SEQ ID NO: 101;
(p) the VH comprises the amino acid sequence set forth in SEQ ID NO: 97 and the VL comprises the amino acid sequence set forth in SEQ ID NO: 89;
(q) the VH comprises the amino acid sequence set forth in SEQ ID NO: 102 and the VL comprises the amino acid sequence set forth in SEQ ID NO: 83;
(r) the VH comprises the amino acid sequence set forth in SEQ ID NO: 102 and the VL comprises the amino acid sequence set forth in SEQ ID NO: 92;
(s) the VH comprises the amino acid sequence set forth in SEQ ID NO: 103 and the VL comprises the amino acid sequence set forth in SEQ ID NO: 83;
(t) the VH comprises the amino acid sequence set forth in SEQ ID NO: 104 and the VL comprises the amino acid sequence set forth in SEQ ID NO: 83;
(u) the VH comprises the amino acid sequence set forth in SEQ ID NO: 105 and the VL comprises the amino acid sequence set forth in SEQ ID NO: 106;
(v) the VH comprises the amino acid sequence set forth in SEQ ID NO: 107 and the VL comprises the amino acid sequence set forth in SEQ ID NO: 108;
(w) the VH comprises the amino acid sequence set forth in SEQ ID NO: 109 and the VL comprises the amino acid sequence set forth in SEQ ID NO: 83;
(x) the VH comprises the amino acid sequence set forth in SEQ NO: 110 and the VL comprises the amino acid sequence set forth in SEQ ID NO: 111;
(y) the VH comprises the amino acid sequence set forth in SEQ ID NO: 112 and the VL comprises the amino acid sequence set forth in SEQ ID NO: 89;
(z) the VH comprises the amino acid sequence set forth in SEQ ID NO: 156 and the VL comprises the amino acid sequence set forth in SEQ ID NO: 157;
(aa) the comprises the amino acid sequence set forth in SEQ ID NO: 0.156 and the VL comprises the amino acid sequence set forth in SEQ ID NO: 83; or
(bb) the VH comprises the amino acid sequence set forth in SEQ ID NO: 88 and the VL comprises the amino acid sequence set forth in SEQ ID NO: 158.

In some embodiments, an anti-TREM-1 antibody useful for the present disclosure is not epitope-steered (i.e., can cross-compete with mAb 0170 and/or mAb 0318 for binding to TREM-1 (human or cynomolgus). In some embodiments, a non-epitope-steered anti-TREM-1 antibody comprises CDRs of a heavy chain variable region selected from the group consisting of SEQ ID NOs: 82, 84, 86, 88, 91, 93, 95, 97, 102, 103, 104, 105, 107, 109, 110, 112, and 156. In some embodiments, the non-epitope-steered anti-TREM-1 antibodies comprise CDRs of a light chain variable region selected from the group consisting of 83, 87, 85, 90, 92, 94, 96, 98, 99, 100, 101, 106, 108, 111, 157, and 158.

In some embodiments, the non-epitope-steered anti-TREM-1 antibodies of the present disclosure comprise heavy chain variable region (VH) CDR1, CDR2, and CDR3 and a light chain variable region (VL) CDR1, CDR2, and CDR3, wherein

(a) the VH CDR1 comprises an amino acid sequence selected from the group consisting of SEQ ID NOs: 74, 113, 118, 122, 128, 136, 139, 142, and 163;
(b) the VH CDR2 comprises an amino acid sequence selected from the group consisting of SEQ ID NOs: 114, 119, 123, 126, 127, 129, 131, 134, 137, 140, 143, 146, 149, and 164;
(c) the VH CDR3 comprises an amino acid sequence selected from the group consisting of SEQ ID NOs: 115, 120, 124, 130, 135, 138, 141, 144, 145, 147, 150, and 165;
(d) the VL CDR1 comprises an amino acid sequence selected from the group consisting of SEQ ID NOs: 116 and 42;
(e) the VL CDR2 comprises an amino acid sequence selected from the group consisting of SEQ ID NOs: 77 and 65; and/or
(f) the VL CDR3 comprises an amino acid sequence selected from the group consisting of SEQ ID NOs: 73, 78, 117, 121, 125, 133, 148, and 166.

In some embodiments, an anti-TREM-1 antibody (non-epitope steered) comprises a VH CDR1, CDR2, and CDR3 and VL CDR1, CDR2, and CDR3, wherein:

(a) the VH CDR1 comprises the amino acid sequence set forth as SEQ ID NOs: 113, the VH CDR2 comprises the amino acid sequence set forth as SEQ ID NO: 114, and the VII CDR3 comprises the amino acid sequence set forth as SEQ ID NO: 115, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 117;
(b) the VH CDR1 comprises the amino acid sequence set forth as SEQ ID NOs: 118, the VH CDR2 comprises the amino acid sequence set forth as SEQ ID NO: 119, and the VH CDR3 comprises the amino acid sequence set forth as SEQ ID NO: 120, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 121;
(c) the VH CDR1 comprises the amino acid sequence set forth as SEQ. ID NOs: 122, the VH CDR2 comprises the amino acid sequence set forth as SEQ ID NO: 123, and the VH CDR3 comprises the amino acid sequence set forth as SEQ ID NO: 124, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 125;
(d) the VH CDR1 comprises the amino acid sequence set forth as SEQ ID NOs: 122, the VH CDR2 comprises the amino acid sequence set forth as SEQ ID NO: 126, and the VR CDR3 comprises the amino acid sequence set forth as SEQ ID NO: 124, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 78;
(e) the VH CDR1 comprises the amino acid sequence set forth as SEQ ID NOs: 122, the VII CDR2 comprises the amino acid sequence set forth as SEQ ID NO: 126, and the VH CDR3 comprises the amino acid sequence set forth as SEQ ID NO: 124, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 117;
(f) the VH CDR1 comprises the amino acid sequence set forth as SEQ ID NO: 122, the VH CDR2 comprises the amino acid sequence set forth as SEQ ID NO: 127, and the VH CDR3 comprises the amino acid sequence set forth as SEQ. ID NO: 124, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 78;
(g) the VH CDR1 comprises the amino acid sequence set forth as SEQ ID NO: 128, the VH CDR2 comprises the amino acid sequence set forth as SEQ ID NO: 129, and the VR CDR3 comprises the amino acid sequence set forth as SEQ ID NO: 130, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 78;
(h) the VH CDR1 comprises the amino acid sequence set forth as SEQ ID NO: 128, the VH CDR2 comprises the amino acid sequence set forth as SEQ ID NO: 131, and the VH CDR3 comprises the amino acid sequence set forth as SEQ ID NO: 132, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 117;
(i) the VII CDR1 comprises the amino acid sequence set forth as SEQ ID NO: 128, the VH CDR2 comprises the amino acid sequence set forth as SEQ ID NO: 131, and the VH CDR3 comprises the amino acid sequence set forth as SEQ ID NO: 132, the CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 133;
(j) the VH CDR1 comprises the amino acid sequence set forth as SEQ ID NO: 128, the VH CDR2 comprises the amino acid sequence set forth as SEQ ID NO: 131, and the VH CDR3 comprises the amino acid sequence set forth as SEQ ID NO: 132, the VL CDR1 comprises the amino acid sequence se forth in SEQ ID NO: 116, the VL CDR2 comprises the amino acid sequence set forth in SEQ ID NO: 77, and the VL CDR3 comprises the amino acid sequence set forth in SEQ ID NO: 78;
(k) the VH CDR1 comprises the amino acid sequence se forth as SEQ ID NO: 122, the VH CDR2 comprises the amino acid sequence set forth as SEQ ID NO: 131, and the VH CDR3 comprises the amino acid sequence set forth as SEQ ID NO: 124, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 117;
(l) the VH CDR1 comprises the amino acid sequence set forth as SEQ ID NO: 74, the VH CDR2 comprises the amino acid sequence sett forth as SEQ ID NO: 134, and the VH CDR3 comprises the amino acid sequence set forth as SEQ ID NO: 135, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence se forth in SEQ ID NO: 117;
(m) the VH CDR1 comprises the amino acid sequence set forth as SEQ ID NO: 136, the VH CDR2 comprises the amino acid sequence set forth as SEQ ID NO: 137, and the VH CDR3 comprises the amino acid sequence sett forth as SEQ ID NO: 138, the VL CDR3 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 117;
(n) the VIA CDR1 comprises the amino acid sequence set forth as SEQ ID NO: 139, the VH CDR2 comprises the amino acid sequence set forth as SEQ ID NO: 140, and the VH CDR3 comprises the amino acid sequence set forth as SEQ. ID NO: 141, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 78;
(o) the VH CDR1 comprises the amino acid sequence set forth as SEQ ID NO: 142, the VH CDR2 comprises the amino acid sequence set forth as SEQ ID NO: 143, and the VH CDR3 comprises the amino acid sequence set forth as SEQ ID NO: 144, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 125;
(p) the VH CDR1 comprises the amino acid sequence set forth as SEQ ID NO: 142, the VH CDR2 comprises the amino acid sequence set forth as SEQ ID NO: 143, and the VH CDR3 comprises the amino acid sequence set forth as SEQ ID NO: 145, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 117;
(q) the VH CDR1 comprises the amino acid sequence set forth as SEQ ID NO: 74, the VH CDR2 comprises the amino acid sequence set forth as SEQ ID NO: 146, and the VH CDR3 comprises the amino acid sequence set forth as SEQ ID NO: 147, the VL CDR1 comprises the amino acid sequence set forth in SEQ ID NO: 116, the VL CDR2, comprises the amino acid sequence set forth in SEQ ID NO: 77, and the VL CDR3 comprises the amino acid sequence set forth in SEQ ID NO: 148;
(r) the VH CDR1 comprises the amino acid sequence set forth as SEQ ID NO: 74, the VH CDR2 comprises the amino acid sequence set forth as SEQ ID NO: 149, and the VH CDR3 comprises the amino acid sequence set forth as SEQ ID NO: 150, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 78;
(s) the CDR1 comprises the amino acid sequence set forth as SEQ ID NO: 163; the VH CDR2 comprises the amino acid sequence set forth as SEQ ID NO: 164, and the VH CDR3 comprises the amino acid sequence set forth as SEQ ID NO: 165, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 166;
(t) the VH CDR1 comprises the amino acid sequence set forth as SEQ ID NO: 163, the VH CDR2 comprises the amino acid sequence set forth as SEQ ID NO: 164, and the VH CDR3 comprises the amino acid sequence set forth as SEQ ID NO: 165, the VL CDR1 comprises the amino acid sequence set forth in SEQ ID NO: 116, the VL CDR2 comprises the amino acid sequence set forth in SEQ ID NO: 77, and the VL CDR3 comprises the amino acid sequence set forth in SEQ ID NO: 117; or
(t) the VH CDR1 comprises the amino acid sequence set forth as SEQ ID NO: 122, the VH CDR2 comprises the amino acid sequence set forth as SEQ ID NO: 126, and the VH CDR3 comprises the amino acid sequence set forth as SEQ ID NO: 124, the VL CDR1 comprises the amino acid sequence set forth in SEQ ID NO: 167, the VL CDR2 comprises the amino acid sequence set forth in SEQ ID NO: 65, and the VL CDR3 comprises the amino acid sequence se forth in SEQ ID NO: 73.

In some embodiments, an anti-TREM-1 antibody that can be used with the present methods comprises CDR and/or variable region sequences that have at least 80% identity (e.g., at least 85%, at least 95%, at least 95%, or at least 99% identity) to the CDR and/or variable region sequences disclosed herein (e.g., Table 9).

In some embodiments, an anti-TREM-1 antibody comprises a heavy chain and a light chain, wherein the heavy chain comprises a VH domain disclosed herein (e.g., those provided in Table 9) fused to a heavy chain constant region described herein (e.g., SEQ ID NOs: 47, 48, 11, or 12). In some embodiments, the anti-TREM-1 antibody disclosed herein comprises a heavy chain and a light chain, wherein the light chain comprises a VL domain disclosed herein (e.g., those provided in Table 9) fused to a light chain constant region described herein (e.g., SEQ ID NO: 35).

In some embodiments, an anti-TREM-1 antibody comprises a heavy chain and a light chain, wherein the heavy chain comprises an amino acid sequence selected from the group consisting of SEQ ID NOs: 168-202, and/or wherein the light chain comprises an amino acid sequence selected from the group consisting of SEQ ID NOs: 203-210.

Heavy and light chains comprising an amino acid sequence that is at least 99%, 98%, 97%, 96%, 95%, 90%, 85%, or 80% identical to any of the heavy or light chains described herein can be used for forming the anti-TREM-1 antibodies having the desired characteristics, e.g., those further described herein.

In some embodiments, an anti-TREM1 antibody comprises a heavy chain constant region, wherein the heavy chain constant region comprises one or more amino acid substitutions selected from the group consisting of K214R, L234A, L235E, G237A, D356E, L358M, and any combination thereof per EU numbering. In some embodiments, an anti-TREM-1 antibody comprises a heavy chain constant region, wherein the heavy chain constant region comprises one or more amino acid substitutions selected from the group consisting of K214R, L234A, L235E, G237A, A3305, P331S, D356E, L358M, and any combination thereof per EU numbering. In some embodiments, an anti-TREM-1 antibody comprises a heavy chain constant region, wherein the heavy chain constant region comprises one or more amino acid substitutions selected from the group consisting of K214R, C226S, C229S, P2385, and any combination thereof per EU numbering. In some embodiments, an anti-TREM-1 antibody comprises a heavy chain constant region, wherein the heavy chain constant region comprises one or more amino acid substitutions selected from the group consisting of S131C, K133R, G137E, G138S, Q196K, I199T, N203D, K214R, C2265, C2295, P2385, and any combination thereof per EU numbering.

In some embodiments, an antibody disclosed herein binds anti-TREM-1 at one or more of the same epitopes as the mAb 0318 antibody. In some embodiments, the anti-TREM-1 antibody is capable of specifically binding (i) at least one amino acid residue selected from the group consisting of the A21. T22, K23, L24, T25, E26, and any combination thereof and (ii) at least one amino acid residue selected from the group consisting of the A49, S50, S51, Q52, K53, A54, W55, Q56, 157, 158, R59, D60, G61, E62, M63, P64, K65, T66, L67, A68, C69, T70, E71, R72, P73, S74, K75, N76, S77, H78, P79, V80, Q81, V82, G83, R84, 185, and any combination thereof and (iii) at least one amino acid residue selected from the group consisting of the C113, V114, 1115, Y116, Q117, P118, P119, and any combination thereof of human TREM-1 (e.g., Isoform 1, SEQ ID NO: 1). See WO 2016/009086.

In some embodiments, an anti-TREM-1 antibody is capable of specifically binding to amino acids D38 to F48 of SEQ ID NO: 1 (human TREM-1), as determined using, e.g., HX-M5 or X-ray diffraction. In some embodiments, the anti-TREM-1 antibody has an epitope comprising one, two, three, four, five, six, seven, or all of the amino acid residues D38, V39, K40, C41, D42, Y43, T44, and L45 of SEQ ID NO: 1 (human TREM-1) and one, two, or all of the amino acid residues selected from the group consisting of the E46, K47, and F48 of SEQ ID NO: 1 (human TREM-1), as determined using, e.g., HX-MS or X-ray diffraction. In certain embodiments, the anti-TREM-1 antibody has an epitope comprising one, two, three, or all of the amino acid residues selected from the group consisting of the D42, E46, D92, and H93 of SEQ ID NO: 1 (human TREM-1), as determined using variants of TREM-1 and surface plasmon resonance.

In some embodiments, the anti-TREM-1 antibody of the present disclosure has an epitope comprising at least the amino acid residues E46 and/or D92 of SEQ ID NO: 1 (human TREM-1), as determined using variants of TREM-1 and surface plasmon resonance. In some embodiments, the anti-TREM-1 antibody comprises one, two, or all of the amino acid residues selected from the group consisting of L31, 186, and V101 of SEQ ID NO: 1 (human TREM-1). In certain embodiments, the anti-TREM-1 antibody is capable of specifically binding a polypeptide comprising amino acid residues E19 to L26 of cynomolgus monkey TREM-1 (SEQ ID NO: 7), as determined using, e.g., HX-MS or X-ray diffraction.

In some embodiments, the anti-TREM-1 antibody is capable of specifically binding human MEM-1, wherein the epitope of the antibody comprises one, two, three, four, five, six, seven, eight, nine, or all of the amino acid residues selected from the group consisting of the V39, K40, C41, D42, Y43, L45, E46, K47, F48, and A49 of SEQ NO: 1.

In some embodiments, the anti-TREM-1 antibody is capable of specifically binding human TREM-1, wherein the epitope of the antibody comprises the D42 of SEQ ID NO: 1. In other embodiments, the anti-TREM-1 antibody is capable of specifically binding human TREM-1, wherein the epitope of the antibody comprises the E46 of SEQ ID NO: 1. In some embodiments, the epitope of the antibody can comprise the V39, C41, D42, Y43, L45 of SEQ ID NO: 1. In further embodiments, the epitope of the antibody can comprise the E46, K47 and A49 of SEQ ID NO: 1. In a specific embodiment, the epitope of the anti-TREM-1 antibody can further comprise the F48 of SEQ ID NO: 1.

In some embodiments, the anti-TREM-1 antibody of the present disclosure comprises mutations, wherein one or more negatively charged residues in the light CDR1 and CDR3 regions of the antibody are substituted with uncharged residues. In some embodiments, the anti-TREM-1 antibody comprises a substitution at one or more of amino acid residues D1, D30, D33, D74, D98, E27, and E97 of SEQ ID NO: 23 with an amino acid residue selected from the group consisting of glycine, alanine, serine, asparagine, glutamine, threonine, cysteine, and tyrosine. These mutations are referred to herein as “charge patch” mutations.

In some embodiments, the anti-TREM-1 antibody of the present disclosure comprises mutations in the Fab-Fab interaction area of SEQ ID NO: 15 to reduce Fab-Fab dimerization. It was previously shown with the mAb 0318 antibody that because antibodies comprise two Fabs, multimerization could impact viscosity. These mutations are referred to as “Fab-Fab interaction” mutations. In certain embodiments, the anti-TREM-1 antibody comprises a mutation at any one of residues Y32, R52, S55, S56, N57, A59, M102, I104 and R106 of SEQ ID NO: 15 or F32, D33, Y34, Y53, R54, and D98 of SEQ ID NO: 23 with an amino acid residue selected from the group consisting of glycine, alanine, serine, asparagine, glutamine, threonine, cysteine, lysine, arginine, tryptophan, histidine and tyrosine.

In some embodiments, the anti-TREM-1 antibody as disclosed herein comprises a mutation at position 32 of SEQ ID NO: 23, wherein the phenylalanine is mutated to an amino acid selected from amino acid residues glycine, serine, threonine, cysteine, alanine, valine, leucine, isoleucine, and methionine. Such mutation is based on the observation that an Ala substitution in position Y90 of SEQ ID NO: 1 improved the affinity of SEQ ID NO: 3 to TREM-1. The Y90 was found to interact with a phenylalanine residue of SEQ ID NO: 23. Mutation of SEQ ID NO: 23 in order to improve the Fab-TREM-1 interaction are referred to as “Fab-TREM-1 interaction” mutations. Provided herein are anti-TREM-1 antibodies whose variable regions are linked (e.g., covalently linked or fused) to an Fc, e.g., an IgG1, IgG2, IgG3 or IgG4 Fc, which can be of any allotype or isoallotype, e.g., for IgG1, G1m, G1m1(a), G1m2(x), G1m3(f), G1m17(z); for IgG2: G2m, G2m23(n); for IgG3: G3m, G3m21(g1), G3m28(g5), G3m11(b0), G3m5(b1), G3m13(b3), G3m14(b4), G3m10(b5), G3m15(s), G3m16(t), G3m6(c3), G3m24(c5), G3m26(u), G3m27(v); and for K: Km, Km1, Km2, Km3 (see, e.g., Jeffries et al. (2009) mAbs 1:1). In some embodiments, the variable regions of the anti-TREM-1 antibodies disclosed herein are linked to an effectorless or mostly effectorless Fc, e.g., IgG1. In some embodiments, the variable regions of the anti-TREM-1 antibodies are linked to an Fc that has reduced binding or is incapable of binding to one or more FcγRs.

In some embodiments, the VH domain of the anti-TREM-1 antibody described herein can be fused to the constant domain of a human IgG (i.e., Fc), e.g., IgG1, IgG2, IgG3, or IgG4, which is either naturally-occurring or modified, e.g., as further described herein. For example, a VH domain can comprise the amino acid sequence of any VH domain described herein fused to a human IgG, e.g., an IgG1, constant region, such as the following wild-type human IgG1 constant domain amino acid sequence:

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVGNVFSCSVMHEALHNHYTQK
SLSLSPGK

or that of an allotypic variant of SEQ ID NO: 9 and have the following amino acid sequences:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHIAKTKPREEQYNSTYRVVSVLTVLHQDWLNGK
EYKCKVSNKALPAPIEKTISKAKGQPREIQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

allotype specific amino acid residues are in bold and underlined).

In some embodiments, the VH domain of the anti-TREM-1 antibody described herein can comprise the amino acid sequence of any VH domain described herein fused to an effectorless constant region, e.g., the following effectorless human IgG4 constant domain amino acid sequences:

(SEQ ID NO: 47
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPSSIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK;

“IgG1.1f,” comprising substitutions L234A, L235E, G237A, A330S and P331S, per EU numbering, which are underlined)
or

(SEQ ID NO: 48
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK;

“IgG1.3f”, comprising substitutions L234A, L235E and G237A, per EU numbering, which are underlined).

For example, an allotypic variant of IgG1 comprises an K97R, D239E, and/or L241M (underlined and bolded above) and numbering according to that in SEQ ID NOs: 46-48. Within the full length heavy region and according to EU numbering, these amino acid substitutions are numbered K214R, D356E, and L358M. In some embodiments, the constant region of an anti-TREM-1 antibody further comprises one or more mutations or substitutions at amino acids L117, A118, G120, A213, and P214 (underlined above) as numbered in SEQ ID NO: 46-48, or L234, A235, G237, A330 and P331, per EU numbering. In further embodiments, the constant region of the anti-TREM-1 antibody comprises one or more mutations or substitutions at amino acids L117A, A118E, G120A, A213S, and P214S of SEQ ID NO: 46-48, or L234A, L235E, G237A, A330S and P331S, per EU numbering. The constant region of the anti-TREM-1 antibody may also comprise one or more mutations or substitutions L117A, A118E and G120A of SEQ ID NO: 9, or L234A, L235E and G237A, per EU numbering.

In some embodiments, the VR domain of the anti-TREM-1 antibodies described herein comprises the amino acid sequence of any WI domain described herein fused to an IgG1 constant domain comprising the following amino acid sequences:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTSPPSPAPELLGGSSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKITPPVLDSDGSFFLYSKLTVDKSRW
QQGNVFSCS

“IgG1-Aba”, comprising substitutions K214R, C226S, C229S, and P238S, per EU numbering, which are underlined); or

(SEQ ID NO: 12
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVEP
KSCDKTHTSPPSPAPELLGGSSVFLFPPKPKDTLMISRTPEVTCVVVDVS
HEDPEVKENWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK;

“IgG4-Aba”, comprising substitutions S131C, K133R, G137E, G1385, Q196K, I199T, N203D, K214R, C226S, C229S, and P238S, per EU numbering, which are underlined).

A VL domain described herein can be fused to the constant domain of a human Kappa or Lambda light chain. For example, a VL domain of an anti-TREM-1 antibody can comprise the amino acid sequence of any VL domain described herein fused to the following human IgG1 kappa light chain amino acid sequence:

(SEQ ID NO: 35)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In certain embodiments, the heavy chain constant region comprises a lysine or another amino acid at the C-terminus, e.g., it comprises the following last amino acids: LSPGK (SEQ ID NO: 151) in the heavy chain. In certain embodiments, the heavy chain constant region is lacking one or more amino acids at the C-terminus, and has, e.g., the C-terminal sequence LSPG (SEQ ID NO: 152) or LSP.

In some embodiments, the variable region of the anti-TREM-1 antibody is linked to an effectorless or mostly effector less Fc. In certain embodiments, the variable region of the anti-TREM-1 antibody is linked to a Fc selected from the group consisting of IgG1.1f, IgG1.3f, IgG1-Aba, and IgG4-Aha, as described herein.

Generally, variable regions described herein can be linked to an Fc comprising one or more modification, typically to alter one or more functional properties of the antibody, such as Fc receptor binding, inflammatory cytokine release, serum half-life, complement fixation, and/or antigen-dependent cellular cytotoxicity. Furthermore, an antibody described herein can be chemically modified (e.g., one or more chemical moieties can be attached to the antibody) or be modified to alter its glycosylation, to alter one or more functional properties of the antibody. Each of these embodiments is described in further detail below. The numbering of residues in the Fc region is that of the EU index of Kabat.

The Fc region encompasses domains derived from the constant region of an immunoglobulin (e.g., IgG1, IgG2, IgG3, IgG4, and other classes such as IgA, IgD, IgE and IgM), including a fragment, analog, variant, mutant or derivative of the constant region. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination.

Ig molecules interact with multiple classes of cellular receptors. For example IgG molecules interact with three classes of Fey receptors (FcγR) specific for the IgG class of antibody, namely FcγRI, FcγRII, and FcγRIII. The important sequences fir the binding of IgG to the FcγR receptors have been reported to be located in the CH2 and CH3 domains. The serum half-life of an antibody is influenced by the ability of that antibody to bind to an Fc receptor (FcR).

In some embodiments, the Fc region of the anti-TREM-1 antibodies is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity.

For example, one can make modifications in the Fc region in order to generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fc. Such Fc region variants will generally comprise at least one amino acid modification in the Fc region. Combining amino acid modifications is thought to be particularly desirable. For example, the variant Fc region can include two, three, four, five, etc. substitutions therein, e.g., of the specific Fc region positions identified herein.

A variant Fc region can also comprise a sequence alteration wherein amino acids involved in disulfide bond formation are removed or replaced with other amino acids. Such removal can avoid reaction with other cysteine-containing proteins present in the host cell used to produce the anti-TREM-1 antibodies described herein. Even when cysteine residues are removed, single chain Fc domains can still form a dimeric Fe domain that is held together non-covalently. In other embodiments, the Fc region can be modified to make it more compatible with a selected host cell. For example, one can remove the PA sequence near the N-terminus of a typical native Fc region, which can be recognized by a digestive enzyme in E. coli such as proline iminopeptidase. In other embodiments, one or more glycosylation sites within the Fc domain can be removed. Residues that are typically glycosylated (e.g., asparagine) can confer cytolytic response. Such residues can be deleted or substituted with unglycosylated residues (e.g., alanine). In other embodiments, sites involved in interaction with complement, such as the C1q binding site, can be removed from the Fc region. For example, one can delete or substitute the EKK sequence of human IgG1. In certain embodiments, sites that affect binding to Fc receptors can be removed, preferably sites other than salvage receptor binding sites. In other embodiments, an Fc region can be modified to remove an ADCC site. ADCC sites are known in the art; see, e.g., Sarmay et al., Molec. Immunol. 29 (5): 633-9 (1992) with regard to ADCC sites in IgG1. Specific examples of variant Fe domains are disclosed, for example, in WO 97/34631 and WO 96/32478.

In some embodiments, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, 322, 330, and/or 331 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In some embodiments, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In some embodiments, one or more amino acid residues within amino acid positions 231 and 239 are altered to thereby alter the ability of the antibody to fix complement. This approach is described further in PCT Publication WO 94/29351 by Bodmer et al.

In some embodiments, the Fc region can be modified to decrease antibody dependent cellular cytotoxicity (ADCC) and/or to decrease the affinity for an Fey receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or X139. Exemplary substitutions include 236A, 239D, 239E, 268D, 267E, 268E, 268F, 324T, 332D, and 332E. Exemplary variants include 239D/332E, 236A/332E, 236A/239D/332E, 268F/3241, 267E/268F, 267E/324T, and 267E/268F/324T. Other modifications for enhancing FcγR and complement interactions include but are not limited to substitutions 298 A, 333A, 334A, 326A, 2471, 339D, 339Q, 280H, 2905, 298D, 298V, 243L, 292P, 300L, 396L, 3051, and 396L. These and other modifications are reviewed in Strohl, 2009, Current Opinion in Biotechnology 20:685-691.

Other Fc modifications that can be made to Fcs are those for reducing or ablating binding to FcγR and/or complement proteins, thereby reducing or ablating Fe-mediated effector functions such as ADCC, ADCP, and CDC. Exemplary modifications include but are not limited substitutions, insertions, and deletions at positions 234, 235, 236, 237, 267, 269, 325, 328, 330, and/or 331 (e.g., 330 and 331), wherein numbering is according to the EU index. Exemplary substitutions include but are not limited to 234A, 235E, 236R, 237A, 267R, 269R, 325L, 328R, 330S, and 331S (e.g., 330S, and 331S), wherein numbering is according to the EU index. An Fc variant can comprise 236R/328R. Other modifications for reducing FcγR. and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl. 2009, Current Opinion in Biotechnology 20:685-691.

Optionally, the Fc region can comprise a on-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; International Publ. Nos. WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925, and WO 06/020114).

The affinities and binding properties of an Fc region for its ligand can be determined by a variety of in vitro assay methods (biochemical or immunological based assays) known in the art including but not limited to, equilibrium methods (e.g., enzyme-linked immunoabsorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In certain embodiments, the anti-TREM-1 antibodies of the present disclosure comprise an Fc that has reduced binding or is incapable of binding to FcγRs. In some embodiments, the anti-TREM-1 antibody has a decreased binding affinity to FcγRI (CD64), FcγRIIA (CD32), FcγRIIB (CD32), FcγRIIIA (CD16a), FcγRIIIB (CD16b), or any combination thereof compared to an antibody comprising a heavy chain consisting of the amino acid sequence as set forth in SEQ ID NO: 30 and a light chain consisting of the amino acid sequence as set forth in SEQ ID NO: 34. In some embodiments, the anti-TREM-1 antibody has a decreased binding affinity to FcγRI (CD64) by at least two fold, at least three fold, at least four fold, at least five fold, at least six fold, at least seven fold, at least eight fold, at least nine fold, or at least 10 fold compared to an antibody comprising a heavy chain consisting of the amino acid sequence as set forth in SEQ ID NO: 30 and a light chain consisting of the amino acid sequence as set forth in SEQ ID NO: 34.

In some embodiments, the anti-TREM-1 antibodies comprise an IgG1. Fc variant comprising: (a) one or more amino acid substitutions selected from the group consisting of L234A, L235E, G237A, and any combination thereof, per EU numbering; (b) one or more amino acid substitutions selected from the group consisting of L234A, L235E, G237A, A3305, P331S, and any combination thereof per EU numbering; (c) one or more amino acid substitutions selected from the group consisting of K214R, C2265, C229S, P238S, and any combination thereof per EU numbering; or (d) one or more amino acid substitutions selected from the group consisting of S131C, K133R, G137E, G138S, Q196K, I199T, N203D, K214R, C2265, C229S, P238S, and any combination thereof per EU numbering.

In some embodiments, the anti-TREM-1 antibody, as disclosed herein, has (a) an IgG1 isotype and comprises one or more amino acid substitutions in the Fc region at an amino acid residue selected from the group consisting of: N297A, N297Q, D270A, D265A, L234A, L235A, C226S, C229S, P238S, E233P, L234V, P238A, A327Q, A327G, P329A, K322A, L234F, L235E, P331S, T394D, A330L, M252Y, S254T, T256E, L328E, P238D, S267E, L328F, E233D, G237D, H268D, P271G, A330R, and any combination thereof, wherein the numbering of the residues is according to EU or Kabat numbering, or comprises an amino acid deletion in the Fc region at a position corresponding to glycine 236; (b) an IgG2 isotype and comprises one or more amino acid substitutions in the Fe region at an amino acid residue selected from the group consisting of: P238S, V234A, G237A, H268A, H268Q, H268E, V309L, N297A, N297Q, A330S, P331S, C232S, C233S, M252Y, S254T, T256E, and any combination thereof, wherein the numbering of the residues is according to EU or Kabat numbering; or (c) an IgG4 isotype and comprises one or more amino acid substitutions in the Fc region at an amino acid residue selected from the group consisting of: E233P, F234V, L234A/F234A, L235A, G237A, E318A, S228P, L236E, S241P, L248E, T394D, M252Y, S254T, T256E, N297A, N297Q, and any combination thereof, wherein the numbering of the residues is according to EU or Kabat numbering. In some embodiments, (a) the Fc region further comprises one or more additional amino acid substitutions at an amino acid residue selected from the group consisting of A330L, L234F; L235E, P331S, and any combination thereof, wherein the numbering of the residues is according to EU or Kabat numbering; (b) the Fc region further comprises one or more additional amino acid substitutions at a position selected from the group consisting of M252Y, S254T, T256E, and any combination thereof, wherein the numbering of the residues is according to EU or Kabat numbering; or (c) the Fe region further comprises a S228P amino acid substitution according to EU or Kabat numbering. See WO 2017/152102.

In certain embodiments, an Fc is chosen that has reduced complement fixation. An exemplary Fe, e.g. IgG1 Fc, with reduced complement fixation has the following two amino acid substitutions: A330S and P331S.

In certain embodiments, an Fc is chosen that has essentially no effector function, i.e., it has reduced binding to FcγRs and reduced complement fixation. An exemplary Fc, IgG1 Fc, that is effectorless comprises the following five mutations: L234A, L235E, G237A, A3305 and P331S.

### IV. Nucleic Acids, Vectors, and Cells

Another aspect described herein pertains to nucleic acid molecules that encode the anti-TREM-1 antibodies described herein. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids other chromosomal DNA. e.g., the chromosomal DNA that is linked to the isolated DNA in nature) or proteins, by standard techniques, including alkaline; SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In some embodiments, the nucleic acid is a cDNA molecule.

Nucleic acids described herein can be obtained using standard molecular biology techniques. For antibodies expressed by hybridomas (e.g. hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

In some embodiments, the nucleic acids described herein are those encoding the VH and VL sequences of the anti-TREM-1 antibodies of the present disclosure. Exemplary DNA sequences encoding the VH and VL sequences are set forth in SEQ ID NOs: 36-39, 226-260 and 40, 261-295 respectively.

A method for making an anti-TREM-1 antibody as disclosed herein can comprise expressing the heavy chain and the light chains in a cell line comprising the nucleotide sequences encoding the heavy and light chains with a signal peptide, SEQ ID NOs: 36-39, 226-260 and 40, 261-295, respectively. Host cells comprising these nucleotide sequences are encompassed herein.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2 and/or CH3). The sequences of human heavy chain constant region genes are known in the art (see, e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, for example, an IgG2 and/or IgG 4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see, e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

Another aspect described herein pertains to cells (e.g., host cells) expressing (e.g., recombinantly) anti-TREM-1 antibodies described herein and related polynucleotides and expression vectors. Provided herein are also vectors comprising polynucleotides comprising nucleotide sequences encoding anti-TREM-1 antibodies or a fragment thereof. In some embodiments, the vectors can be used for recombinantly expressing anti-TREM-1 antibodies described herein in host cells, e.g., in mammalian cells. In some embodiments, the vectors can be used for gene therapy.

Suitable vectors for the disclosure include expression vectors, viral vectors, and plasmid vectors. In some embodiments, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

Expression vectors of the disclosure can include polynucleotides encoding the antibody or antigen binding portion thereof described herein. In some embodiments, the coding sequences for the antibody or antigen binding portion thereof is operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired antibody or antigen binding portion thereof.

Viral vectors include, but are not limited to, nucleic acid sequences from the following viruses: retrovirus, such as Moloney murine leukemia virus, Harvey murine sarcoma virus, murine mammary tumor virus, and Rous sarcoma virus lentivirus; adenovirus; adeno-associated virus; SV40-type viruses; polyomnaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991).

In some embodiments, the virus is an adeno-associated virus, a double-stranded DNA virus. The adeno-associated virus can be engineered to be replication-deficient and is capable of infecting a wide range of cell types and species. It further has advantages such as heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

### V. Immunoconjugates

The present disclosure also provides immunoconjugates comprising any of the anti-TREM-1 antibodies disclosed herein. In some embodiments, the immunoconjugate comprises an antibody or an antigen binding portion linked to an agent. In some embodiments, the immunoconjugate comprises a bispecific molecule disclosed herein linked to an agent (e.g., as therapeutic agent or a diagnostic agent).

For diagnostic purposes, appropriate agents are detectable labels that include radioisotopes, for whole body imaging, and radioisotopes, enzymes, fluorescent labels and other suitable antibody tags for sample testing. The detectable labels that can be linked to any anti-TREM-1 antibody described herein can be any of the various types used currently in the field of in vitro diagnostics, including particulate labels including metal sols such as colloidal gold; isotopes such as I¹²⁵ or Tc⁹⁹ presented for instance with a peptidic chelating agent of the N₂S₂, N₃S or N₄ type, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1.3,7}decan}-4-yl) phenyl phosphate (CSPD), as well as CDP and CDP-STAR® or other luminescent substrates well-known to those in the art, for example the chelates of suitable lanthanides such as Terbium(III) and Europium(III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter; and the like, all in accordance with standard practice.

In some embodiments, conjugation methods result in linkages which are substantially (or nearly) non-immunogenic, e.g., peptide- (i.e., amide-), (sterically hindered), disulfide-, hydrazone-, and ether linkages. These linkages are nearly non-immunogenic and show reasonable stability within serum (see, e.g., Senter, P. D., Curr. Opin. Chem. Biol. 13 (2009) 235-244; WO 2009/059278; WO 95/17886).

Depending on the biochemical nature of the moiety and the antibody, different conjugation strategies can be employed. In case the moiety is naturally-occurring or recombinant of between 50 to 500 amino acids, there are standard procedures in text books describing the chemistry for synthesis of protein conjugates; which can be easily followed by the skilled artisan (see, e.g, Hackenberger, C. P. R., and Schwarzer, D., Angew. Chem. Int. Ed. Engl. 47 (2008) 10030-10074). In some embodiments the reaction of a maleinimido moiety with a cysteine residue within the antibody or the moiety is used. This is an especially suited coupling chemistry in case e.g., a Fab or Fab′-fragment of an antibody is used. Alternatively, in some embodiments, coupling to the C-terminal end of the antibody or moiety is performed. C-terminal modification of a protein; e.g., of a Fab-fragment, can be performed as described (Sunbul, M. and Yin, J., Org. Biomol. Chem. 7 (2009) 3361-3371).

In general, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, Taki, et al., Prot. Eng. Des. Set. 17 (2004) 119-126; Gamier, A, et al. Chem. Biol. 15 (2008) 128-136; and Protease-catalyzed formation of C N bonds is used by Bordusa, F. Highlights in Bioorganic Chemistry (2004) 389-403). Site specific reaction and covalent coupling can also be achieved by the selective reaction of terminal amino acids with appropriate modifying reagents.

The reactivity of an N-terminal cysteine with benzonitrils (see Ren, H. et al. Angew. Chem. Int. Ed. Engl. 48 (2009) 9658-9662) can be used to achieve a site-specific covalent coupling.

Native chemical ligation can also rely on C-terminal cysteine residues (Taylor, E. Vogel; Imperiali, B, Nucleic Acids and Molecular Biology (2009), 22 (Protein Engineering), 65-96).

US6437095 B1 describes a conjugation method which is based on the faster reaction of a cysteine within a stretch of negatively charged amino acids with a cysteine located in a stretch of positively charged amino acids.

The moiety can also be a synthetic peptide or peptide mimic. In case a polypeptide is chemically synthesized, amino acids with orthogonal chemical reactivity can be incorporated during such synthesis (see, e.g., de Graaf, A. J. et al., Bioconjug. Chem. 20 (2009) 1281-1295). Since a great variety of orthogonal functional groups is at stake and can be introduced into a synthetic peptide, conjugation of such peptide to a linker is standard chemistry.

In order to obtain a mono-labeled polypeptide, the conjugate with 1:1 stoichiometry can be separated by chromatography from other conjugation side-products. This procedure can be facilitated by using a dye labeled binding pair member and a charged linker. By using this kind of labeled and highly negatively charged binding pair member, mono conjugated polypeptides are easily separated from non-labeled polypeptides and polypeptides whiCh carry more than one linker, since the difference in charge and molecular weight can be used for separation. The fluorescent dye can be useful for purifying the complex from un-bound components, like a labeled monovalent binder.

In some embodiments, the moiety attached to an anti-TREM-1 antibody is selected from the group consisting of a binding moiety, a labeling moiety, and a biologically active moiety.

Anti-TREM-1 antibodies described herein can also be conjugated to a therapeutic agent to form an immunoconjugate such as an antibody-drug conjugate (ADC). Suitable therapeutic agents include antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. In some embodiments, the linker is a peptidyl linker such as Val-Cit, Ala-Val, Val-Ala-Val, Lys-Lys, Pro-Val-Gly-Val-Val (SEQ ID NO: 49), Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038658; WO 07/051081; WO 07/059404; WO 08/083312; and WO 08/103693; U.S. Patent Publications 20060024317; 20060004081; and 20060247295.

Anti-TREM-1 antibodies, e.g., those described herein, can also be used for detecting TREM-1, such as human TREM-1, e.g., human TREM-1 in tissues or tissue samples. The antibodies can be used, e.g., in an ELISA assay or in flow cytometry. In some embodiments, an anti-TREM-1 antibody is contacted with cells, e.g., cells in a tissue, for a time appropriate for specific binding to occur, and then a reagent, e.g., an antibody that detects the anti-TREM-1 antibody, is added. Exemplary assays are provided in the Examples. The anti-TREM-1 antibody can be a fully human antibody, or it can be a chimeric antibody, such as an antibody having human variable regions and murine constant regions or a portion thereof. Exemplary methods for detecting TREM-1, e.g., human TREM-1, in a sample (cell or tissue sample) comprise (i) contacting a sample with an anti-TREM-1 antibody, for a time sufficient for allowing specific binding of the anti-TREM-1 antibody to TREM-1 in the sample, and (2) contacting the sample with a detection reagent, e.g., an antibody, that specifically binds to the anti-TREM-1 antibody, such as to the Fc region of the anti-TREM-1 antibody, to thereby detect TREM-1 bound by the anti-TREM-1 antibody. Wash steps can be included after the incubation with the antibody and/or detection reagent. Anti-TREM-1 antibodies for use in these methods do not have to be linked to a label or detection agents, as a separate detection agent can be used.

Other uses for anti-TREM-1 antibodies, e.g., as monotherapy or combination therapy, are provided elsewhere herein, e.g., in the section pertaining to combination treatments.

### VI. Bispecific Molecules

Anti-TREM-1 antibodies described herein can be used for forming bispecific molecules. An anti-TREM-1 antibody, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. For example, an anti-TREM-1 antibody can be linked to an antibody or scFv that binds specifically to any protein that can be used as potential targets for combination treatments, such as the proteins described herein (e.g., antibodies to IP-10 or TNF-α). The antibody described herein can in fact be derived or linked to more than one other functional molecule to generate multispecific molecules that bind to more than two different binding sites and/or target molecules such multispecific molecules are also intended to be encompassed by the term “bispecific molecule” as used herein. To create a bispecific molecule described herein, an antibody described herein can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, provided herein are bispecific molecules comprising at least one first binding specificity for TREM-1 and a second binding specificity for a second target epitope. In some embodiments described herein in which the bispecific molecule is multispecific, the molecule can further include a third binding specificity.

In some embodiments, the bispecific molecules described herein comprise as a binding specificity at least one antibody, or an antibody fragment thereof, including, e.g., an Fab, Fab′, F(ab′)2, Fv, or a single chain Fv (scFv). The antibody can also be a light chain or heavy chain dimer, or any minimal fragment thereof such as a Fv or a single chain construct as described in Ladner et al. U.S. Pat. No. 4,946,778.

While human monoclonal antibodies are preferred, other antibodies which can be employed in the bispecific molecules described herein are murine, chimeric and humanized monoclonal antibodies.

The bispecific molecules described herein can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide. N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see, e.g., Karpovsky et al. (1984) J. Exp. Med. 160: 1686; Liu, M A et al. (1985) Proc. Natl. Acad. Sd. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Some conjugating agents are SATA and suffo-SMC, both available from Pierce Chemical Co. (Rockford, Ill.).

When the binding specificities are antibodies, they can be conjugated via sulfhydryl bonding of the C-terminus hinge regions of the two heavy chains. In some embodiments, the hinge region is modified to contain an odd number of sulfhydryl residues; preferably one, prior to conjugation.

Alternatively, both binding specificities can be encoded in the same vector and expressed and assembled in the same host cell. This method is particularly useful where the bispecific molecule is a mAb×mAb, mAb×Fab, mAb×(scFv)₂, Fab×F(ab′)₂ or ligand×Fab fusion protein. A bispecific antibody can comprise an antibody comprising an scFv at the C-terminus of each heavy chain. A bispecific molecule described herein can be a single chain molecule comprising one single chain antibody and a binding determinant, or a single chain bispecific molecule comprising two binding determinants. Bispecific molecules can comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g. an antibody) specific for the complex of interest.

### VII. Kits

Provided herein are kits comprising one or more anti-TREM-1 antibodies described herein, or antigen-binding portions thereof, bispecific molecules, or immunoconjugates thereof. In some embodiments, provided herein is a pharmaceutical pack or kit comprising one or more containers filled with one or more of the ingredients of the pharmaceutical compositions described herein, such as one or more antibodies provided herein or an antigen-binding portion thereof, optional an instructing for use. In some embodiments, the kits contain a pharmaceutical composition described herein and any prophylactic or therapeutic agent, such as those described herein.

### VIII. Compositions and Formulations

Further provided herein are compositions (e.g., pharmaceutical compositions) and formulations comprising one or more of the anti-TREM-1 antibodies (including polynucleotides, vectors, and cells that encode and/or express the anti-TREM-1 antibodies) disclosed herein. For example, in one embodiment, the present disclosure provides a pharmaceutical composition comprising one or more anti-TREM-1 antibodies as disclosed herein, formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, can be coated in a material to protect the compound from the action of acids and other natural conditions that can inactivate the compound.

Accordingly, one object of the present disclosure is to provide a pharmaceutical formulation, which improves the stability of the anti-TREM-1 antibodies and thus, allows for their long-term storage. In some embodiments, the pharmaceutical formulation disclosed herein comprises: (a) an anti-TREM-1 antibody; (b) a buffering agent; (c) a stabilizing agent; (d) a salt; (e) a bulking agent; and/or (f) a surfactant. In some embodiments, the pharmaceutical formulation is stable for at least 1 month, at least 2 months, at least 3 months, at least 6 months, at least 1 year, at least 2 years, at least 3 years, at least 5 years or more. In some embodiments, the formulation is stable when stored at 4° C., 25° C., or 40° C.

Buffering Agent

Buffering agents useful for the present invention can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also dependent on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between about 2 and about 10, or between about 4 and about 8.

Stabilizing Agent

Stabilizing agents are added to a pharmaceutical product in order to stabilize that product. Such agents can stabilize proteins in a number of different ways. Common stabilizing agents include, but are not limited to, amino acids such as glycine, alanine, lysine, arginine, or threonine, carbohydrates such as glucose, sucrose, trehalose, raffinose, or maltose, polyols such as glycerol, mannitol, sorbitol, cyclodextrins or destrans of any kind and molecular weight, or PEG. In one aspect of the invention, the stabilizing agent is chosen in order to maximize the stability of FIX polypeptide in lyophilized preparations. In certain embodiments, the stabilizing agent is sucrose and/or arginine.

Bulking Agent

Bulking agents can be added to a pharmaceutical product in order to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactant

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

In some embodiments, the pharmaceutical formulation of the present disclosure comprises:

(a) about 0.25 mg/mL to 250 mg/mL (e.g., 10 to 200 mg/mL) of an anti-TREM-1 antibody;
(b) about 20 mM histidine;
(c) about 150 mM sucrose;
(d) about 25 mM arginine; and
(e) about 50 mM NaCl.

The formulation can further comprise one or more of a buffer system, a preservative, a tonicity agent, a chelating agent, a stabilizer and/or a surfactant, as well as various combinations thereof. The use of preservatives, isotonic agents; chelating agents, stabilizers and surfactants in pharmaceutical compositions is well-known to be skilled person. Reference may be made to Remington: The Science and Practice of Pharmacy, 19th edition, 1995.

In some embodiments, the pharmaceutical formulation is an aqueous formulation.

Such a formulation is typically a solution or a suspension, but may also include colloids, dispersions, emulsions, and multi-phase materials. The term “aqueous formulation” is defined as a formulation comprising at least 50% w/w water. Likewise, the term “aqueous solution” is defined as a solution comprising at least 50% water, and the term “aqueous suspension” is defined as a suspension comprising at least 50% w/w water.

In some embodiments, the pharmaceutical formulation is a freeze-dried formulation, to which the physician or the patient adds solvents and/or diluents prior to use.

Pharmaceutical compositions described herein also can be administered in combination therapy, i.e., combined with other agents. For example, the combination therapy can include an anti-TREM-1 antibody described herein combined with at least one other therapeutic agent. Examples of therapeutic agents that can be used in combination therapy can include other compounds, drugs, and/or agents used for the treatment of a disease or disorder (e.g., an inflammatory disorder). Such compounds, drugs, and/or agents can include, for example, anti-inflammatory drugs or antibodies that block or reduce the production of inflammatory cytokines. In some embodiments, therapeutic agents can include an anti-IP-10 antibody, an anti-TNF-α antibody (e.g., adalimumab (HUMIRA®), golimumab (SIMPONI®), infliximab (REMICADE®), certolizumab pegol (CIMZIA®)), interferon beta-1a (e.g., AVONEX®, REBIF®), interferon beta-1b (e.g., BETASERON®, EXTAVIA®), glatiramer acetate (e.g., COPAXONE®, GLATOPA®), mitoxantrone (e.g., NOVANTRONE®), non-steroidal anti-inflammatory drugs (NSAIDs), analgesics, corticosteroids, and combinations thereof.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein can also include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition can comprise a preservative or can be devoid of a preservative. Supplementary active compounds can be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms described herein are dictated by and directly dependent on (a) the unique characteristics of the active compound and the particular therapeutic effect to be achieved, and (b) the limitations inherent in the art of compounding such an active compound for the treatment of sensitivity in individuals.

For administration of an anti-TREM-1 antibody, e.g., described herein, the dosage ranges from about 0.0001 to 100 mg/kg, and more usually 0.01 to 5 or 10 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Exemplary dosage regimens for an anti-TREM-1 antibody described herein include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some embodiments, the anti-TREM-1 antibody is administered at a flat dose (flat dose regimen). In other embodiments, the anti-TREM-1 antibody is administered at a fixed dose with another antibody. In certain embodiments, the anti-TREM-1 antibody is administered at a dose based on body weight.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

An antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

Actual dosage levels of the active ingredients in the pharmaceutical compositions described herein can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A composition described herein can be administered via one or more routes of administration using one or more of a variety of methods known in the art. As will be appreciated by the skilled artisan, the route and/or mode of administration will vary depending upon the desired results. Routes of administration for the anti-TREM-1 antibodies described herein can include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody described herein could potentially be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a particular embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules for use with anti-TREM-1 antibodies described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In some embodiments, the anti-TREM-1 antibodies described herein can be formulated to ensure proper distribution in vivo. For example, the blood-brain barrier (BBB) excludes many highly hydrophilic compounds. To ensure that the therapeutic compounds described herein cross the BBB (if desired, e.g., for brain cancers), they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa, et al., (1988) Biochem. Biophys. Res. Common. 153: 1038); antibodies (P. G. Bloeman et al. (1995) FEBS Lett. 357: 140; M. Owais et al. (1995) Antimicrob. Agents Chemother. 39: 180); surfactant protein A receptor (Briscoe et al. (1995) Am. J Physiol. 1233: 134); p120 (Schreier et al. (1994) J. Biol. Chem. 269:9090); see also K. Keinanen; M. L. Laukkanen (1994) FEBS Lett. 346: 123; J. J. Killion; I. J. Fidler (1994) Immunomethods 4:273.

The following examples are offered by way of illustration and not by way of limitation. The contents of all references cited throughout this application are expressly incorporated herein by reference.

### EXAMPLES

### Example 1: Comparison of TREM-1 Activation with PGLYRP1 Alone or in Combination with Different PGN

With the identification of PGLYRP1 as the TREM-1 ligand, the differential expression of genes following TREM-1 receptor ligation in both monocytes and neutrophils was evaluated. Monocytes and neutrophils were isolated from whole blood peripheral blood mononuclear cells (PBMCs) of healthy human donors. To isolate the cells, whole blood from healthy donors was layered over a ficoll gradient. Monocytes were extracted from the peripheral blood mononuclear cell (PBMC) layer, while neutrophils were isolated from the red blood cell (RBC) containing layer. Next, the neutrophil layer was resuspended in HETASEP™ solution (Stem Cell Technologies) and incubated at 37° C. for 1 hour to precipitate the RBC. Isolated neutrophils were then resuspended in sterile water for 30 seconds, followed by addition of 0.611 KCl. For monocyte isolation, ficoll purified PBMC's were washed, and then, monocytes were purified using the EASYSEP™ human monocyte enrichment kit without CD16 depletion (Stem Cell Technologies).

Once isolated, monocytes and neutrophils (1×10⁶ cells/well) were plated onto a 24-well plate. Then, the cells were stimulated with the following: (i) no stimulation, (ii) PGLYRP1 (soluble), (iii) PGLYRP1 (plate-bound), or (iv) PGLYRP1+PGN. Previous studies had demonstrated that PGLYRP1 in combination with bacterial component peptidoglycan (PGN) can efficiently induce TREM-1 signaling. PGN derived from Staphylococcus aureus (PGN-SA), Escherichia coli (PGN-EK), Bacillus subtilus (PGN-BS), or lacking TLR2 activity (PGN-ECndss) was used. Recombinant PGRP was plate coated on Nunc Maxisorp plate at 5 ug/ml O/N. Plates were washed and PGN was added to a final concentration of 10 ug/ml with purified monocytes and neutrophils. Cultures were incubated overnight at 37° C., 5% CO2 and media removed to measure cytokine production. To measure TREM-1 activity, the levels of TNF-α produced by the monocytes and neutrophils were measured by AlphaLISA assay.

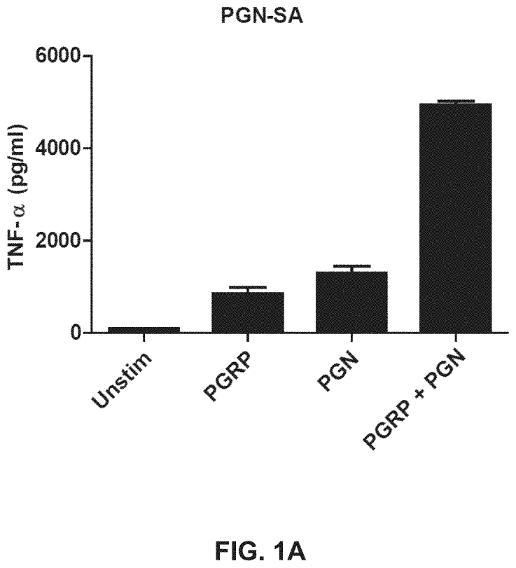

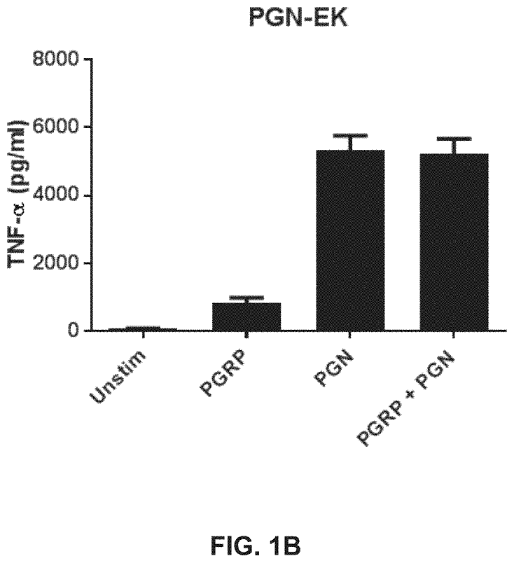

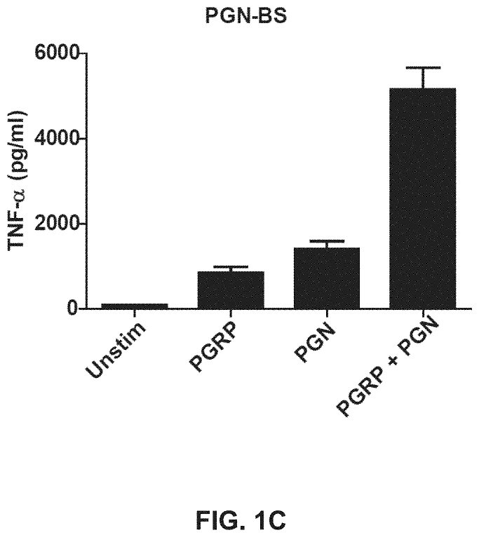

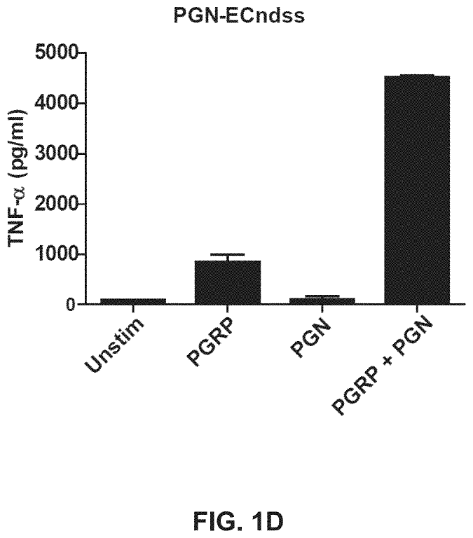

Soluble PGLYRP1 was unable to stimulate TNF-α expression in purified human monocytes (data not shown) while plate-immobilized PGLYRP1 induced TNF-α production (FIG. 1A). PGN derived from Staphylococcus aureus (PGN-SA) induced TNF-α secretion by human monocytes to a similar levels as plate-bound PGLYRP1, while addition of PGLYRP1 and PGN-SA demonstrated synergy as shown in FIG. 1A. Similar PGN-dependent monocyte activation was observed with PGN extracted from Escherichia coli (PGN-EK) and Bacillus subtilus (PGN-BS) through TLR2 receptor ligation. See FIGS. 1B and 1C. PGN-ECndss (i.e., lacking TLR2 activity) was unable to induce TNF-α production alone but potently augmented PGLYRP1 mediated response (FIG. 1D). Thus, to reduce background from PGN signaling, PGN-ECndss was used for subsequent examples.

### Example 2: Evaluation of TREM-1 Gene Signature after TREM-1 Ligation

To generate a robust TREM-1 gene signature via gene expression analysis, the transcriptome profiling pattern following TREM-1 ligation was evaluated. Briefly, peripheral blood monocytes and neutrophils were isolated from healthy donors and plated onto a 24-well plate as described above in Example 1. The isolated monocytes and neutrophils were stimulated with the following for 6 or 24 hours: (i) no stimulation, (ii) PGLYRP1 alone (PGRP), (iii) PGN-ECndss alone (PGN), or (iv) a combination of PGLYRP1 and PGN-ECndss (PGN+PGRP), PGN-ECndss, PGN-EK, PGN-SA and PGN-BS were obtained through Invivogen. To ensure that any genes induced were specific to TREM-1 pathway engagement, some of the monocytes and neutrophils were stimulated with PGLYRP1+PGN-ECndss in the presence of a TREM-1 blocking antibody or an isotype antibody control. After stimulation, RNA was isolated from the cells using RNeasy Micro Kit (Qiagen, Valencia, Calif.). RNA quality was monitored using Agilent 2100 Bioanalyzer (Agilent Technologies, Palo Alto, Calif.) and RNA quantity was measured with NanoDrop (NanoDrop Technologies), 50 ng of RNA was amplified and labeled with Nugen WT-Pico Ovation System and Encore Biotin Module assay (NuGEN Technologies Inc.). Labeled cRNA/cDNA were hybridized on Affymetrix GeneChip Human Genome U219 Array Plate (Affymetrix) and processed according to manufacturer's recommendation.

A custom CDF BrainArray was used to annotate Affy based mRNA expression data (.cel files from U219 and U133Plus platforms), with Entrez gene ID as the unit. Log 2 RMA was used to normalize expression values. To identify differentially expressed genes and construct TREM-1 module, a linear mixed model was fitted with ligand stimulation or antibody treatment as fixed effect, and donor as random effect. Single sample gene set enrichment (ssGSEA) was used to apply gene module in tissue or blood mRNA profiling data from patients and derive a quantitative score representing enrichment of the gene module. Bioinformatics and statistical analyses e.g. multiple regression to identify biomarkers associated with TREM-1 signature, were performed in R with relevant Bioconductor packages (e.g. LIMMA) and Omicsoft ArrayStudio. Visualization is performed in R ggplot framework and Omicsoft Array Studio.

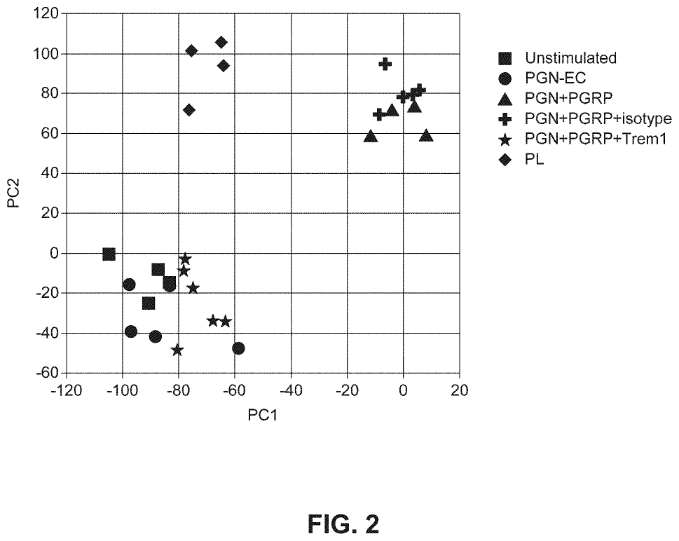

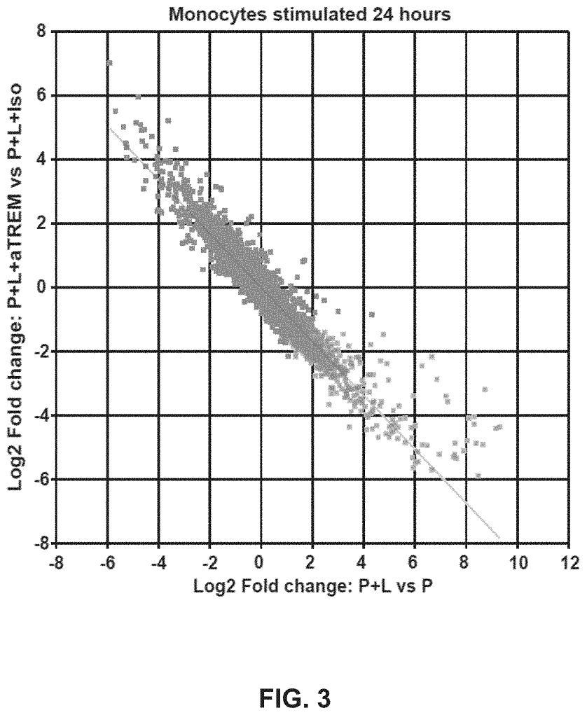

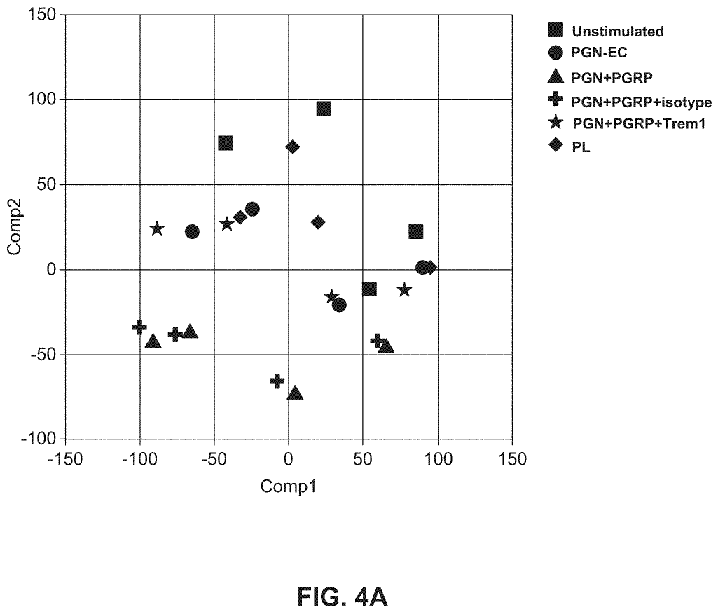

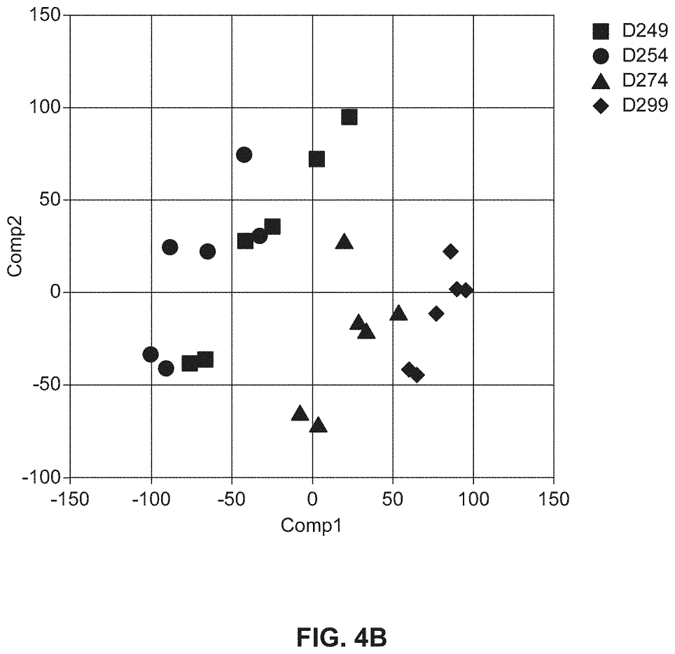

As shown in FIG. 2, the principal component analysis showed that in monocytes at 24 h, PGN-ECndss alone (Media vs P) did not significantly enhance or inhibit global gene expression pattern compared to unstimulated. However, PGN-ECndss together with PGLYRP1 (PGN PG-RP) induced a substantial number of genes, making a separate cluster shown in PCA analysis (upper right quadrant, FIG. 2), PGLYRP1 alone enhanced a subset of genes which was further augmented when PGN-ECndss was added. Addition of an anti-TREM-1 blocking antibody (but not an isotype control antibody) inhibited genes induced by PGLYRP1+PGN-ECndss treatment, confirming that the induced genes are specific to the TREM-1 pathway (lower left quadrant, FIG. 2; see also FIG. 3). In neutrophils, we observed less robust expression pattern changes on PGLYRP1+PGN-ECndss stimulation in 6 hours, and high donor to donor variation as shown in FIGS. 4A and 4B. Therefore, gene expression in monocytes after 24 his of stimulation was used as the primary source for generating TREM-1 module.

Selection of genes for the TREM-1 module was based on the following: 1) significant upregulation upon PGN-ECndss+PGLYRP1 stimulation vs PGN-ECndss alone (P+L vs P, fold change >4, FDR <0.05, 292 genes in total); 2) significant down regulation upon PGN-ECndss+PGLYRP1+TREM-1 antibody vs PGN-ECndss+PGLYRP1 (P-+L) (P+L+TREM-1 vs P+L, fold change <−2, FDR <0.05, 286 out of 292 genes in 1) and 3) non-significant differential expression in PGN-ECndss stimulation (P vs media, fold FDR >0.05, 180 out of 286 genes in 2) and non-significant differential expression in PGN-ECndss+PGLYRP1 vs PGN-ECndss+isotype controls (P+L vs P+L+iso, FDR >0.05, all 180 genes).

A total of 180 genes passed the defined selection criteria. GO functional annotation of genes in the TREM-1 module showed enrichment in extracellular space and plasma membrane localized proteins (Table 4, below) (LOD=logarithm of odds; pVal=probability value; Pcor=probability of correlation). Metacore pathway enrichment analysis of the TREM-1 module includes included gene networks involved in chemotaxis, inflammatory responses (THL7-derived and innate inflammation), and cell proliferation (Table 5, below) (FDR=false discovery rate). Of the 180 gene module, the top 20 genes ordered by the magnitude of PGLYRP1 specific stimulation are listed in Table 6 (below).

TABLE 4

| Term | Description | LOD | Pval | Pcor |
|---|---|---|---|---|
| GO:0005615 | Extracellular | 34.8 | 1.51E−35 | 3.94E−33 |
| | space | | | |
| GO:0016020 | Membrane | 18.1 | 2.27E−19 | 5.91E−17 |
| GO:0044444 | Cytoplasmic part | 15.8 | 1.95E−16 | 5.08E−14 |
| GO:0009986 | Cell surface | 11.4 | 4.76E−12 | 1.24E−09 |
| GO:0031012 | Extracellular | 10.2 | 6.56E−11 | 1.71E−08 |
| | matrix | | | |
| GO:0031982 | Vesicle | 9.12 | 1.07E−09 | 2.79E−07 |
| GO:0012505 | Endomembrane | 6.81 | 2.33E−07 | 6.05E−05 |
| | system | | | |
| GO:0044422 | Organelle part | 5.84 | 2.62E−06 | 6.81E−04 |
| GO:0005634 | Nucleus | 5.19 | 1.17E−05 | 0.00303 |
| GO:0031252 | Cell leading edge | 3.88 | 0.000158 | 0.041 |

TABLE 5

| Metacore Pathways | Pval | FDR | NMember | NPathway |
|---|---|---|---|---|
| Immune response Thl7-derived | 1.22E−06 | 1.44E−04 | 10 | 98 |
| cytokines | | | | |
| Chemotaxis | 3.83E−06 | 2.26E−04 | 11 | 137 |
| Cell adhesion Leucocyte chemotaxis | 6.74E−06 | 2.65E−04 | 13 | 205 |
| Inflammation MIF signaling | 3.03E−05 | 8.93E−04 | 10 | 140 |
| Proliferation Positive regulation cell | 7.27E−05 | 1.72E−03 | 12 | 221 |
| proliferation | | | | |
| Cell cycle G1-S growth factor regulation | 1.05E−04 | 2.07E−03 | 11 | 137 |
| Cell adhesion Platelet-endothelium- | 1.89E−04 | 3.19E−03 | 10 | 174 |
| leucocyte interactions | | | | |
| Inflammation IL-10 anti-inflammatory | 2.4E−04 | 3.54E−03 | 7 | 87 |
| response | | | | |
| Inflammation Innate inflammatory | 1.10E−03 | 1.44E−02 | 9 | 180 |
| response | | | | |

TABLE 6

| | | | | Log2Fc aTREM1 | |
|---|---|---|---|---|---|
| Gene | Inhibition | Log2Fc.P + L vs P | FDR | vs isotype | Description |
| MMP1 | 95.28% | 9.3108 | 9.73E−21 | −4.36 | Matrix |
| | | | | | metallopeptidase |
| | | | | | 1 |
| IL6 | 95.44% | 9.1585 | 1.93E−17 | −4.4022 | Interleukin 6 |
| CCL20 | 89.18% | 8.7289 | 2.29E−14 | −3.1808 | Chemokine (C-C |
| | | | | | motif) ligand 20 |
| TNFSF15 | 96.91% | 8.6506 | 1.32E−18 | −4.9092 | Tumor necrosis |
| | | | | | factor |
| | | | | | superfamily, |
| | | | | | member 15 |
| MMP10 | 98.58% | 8.4687 | 1.43E−23 | −5.8835 | Matrix |
| | | | | | metallopeptidase |
| | | | | | 10 |
| CCL1 | 96.86% | 8.3941 | 1.19E−23 | −4.8681 | Chemokine (C-C |
| | | | | | motif) ligand 1 |
| PTGS2 | 94.18% | 8.3159 | 3.68E−21 | −4.0304 | Prostaglandin- |
| | | | | | endoperoxide |
| | | | | | synthase 2 |
| MMP7 | 95.10% | 8.3155 | 1.09E−20 | −4.2644 | Matrix |
| | | | | | metallopeptidase |
| | | | | | 7 |
| SCG5 | 94.45% | 8.089 | 3.04E−25 | −4.085 | Secretogranin V |
| INHBA | 96.72% | 8.0408 | 3.99E−22 | −4.7757 | Inhibin, beta A |
| IL36G | 97.50% | 7.9082 | 3.12E−21 | −5.1068 | Interleukin 36, |
| | | | | | gamma |
| OCSTA | 98.02% | 7.6205 | 6.36E−22 | −5.3321 | Osteoclast |
| MP | | | | | stimulatory |
| | | | | | transmembrane |
| | | | | | protein |
| TFPI2 | 97.08% | 7.5662 | 6.56E−24 | −4.8659 | Tissue factor |
| | | | | | pathway |
| | | | | | inhibitor 2 |
| F3 | 97.83% | 7.5493 | 1.22E−17 | −5.2146 | Coagulation |
| | | | | | factor III |
| RGS16 | 97.99% | 7.5197 | 2.85E−23 | −5.2955 | Regulator of G- |
| | | | | | protein signaling |
| | | | | | 16 |
| ILIRN | 92.14% | 7.2775 | 9.36E−19 | −3.5635 | Interleukin1 |
| | | | | | receptor |
| | | | | | antagonist |
| CXCL3 | 91.08% | 7.2144 | 4.13E−21 | −3.3912 | Chemokine (C- |
| | | | | | X-Cmotif) |
| | | | | | ligand 3 |
| IL23A | 98.10% | 6.9593 | 6.56E−22 | −5.2173 | Interleukin 23, |
| | | | | | alpha subunit |
| | | | | | P19 |
| ILIA | 87.02% | 6.856 | 4.22E−15 | −2.8645 | Interleukin 1, |
| | | | | | alpha |
| IL24 | 99.05% | 6.6649 | 5.23E−21 | −5.6969 | Interleukin 24 |

### Example 3: Comparison of TREM-1 Gene Signature Using Different TREM4 Agonists

To understand the overlap in gene expression between an agonistic anti-TREM-1 antibody and the natural TREM-1 ligand (PGLYRP1), gene modules generated following ligand (891 genes, P+L, vs P, fold change >2, FDR (0.05) and agonist antibody (331 genes, agTREM-1 vs isotype, fold change >2, FDR<0.05) stimulation were evaluated. Agonist antibody was described in Dower K et. al., Journal of immunology 180: 3520-3534 (2008).

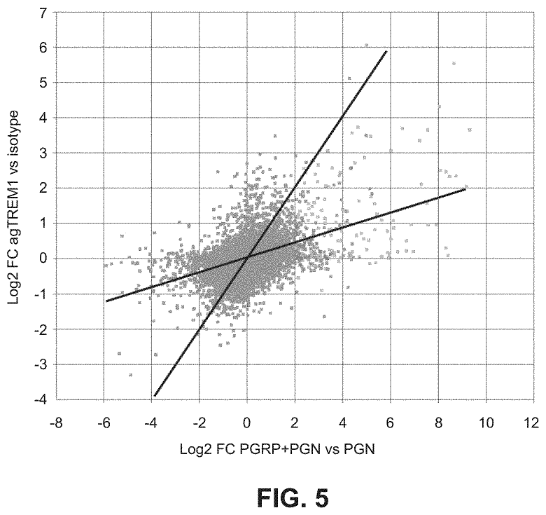

As shown in FIG. 5, only a small subset of genes overlapped between these two TREM-1 agonists.

### Example 4: Evaluation of TREM-1 Gene Signature in Peripheral Blood Mononuclear Cells (PBMCs)

One of the critical components of clinical development is development of robust pharmacodynamic (PD) assay that can be measured in the clinical trial setting. To this end, whether TREM-1 specific gene changes can be recapitulated in PBMCs, which are easier to obtain in the clinic than monocytes, was evaluated. Briefly, PBMCs from healthy human donors were isolated by ficoll purification. PBMCs were then plated onto a 24-well plate (1×10⁶ cells/well) and stimulated for 24 hours using the following: (i) media alone (i.e., no stimulation), (ii) PGN-ECndss alone, (iii) PGN-ECndss PGRP, and (iv) PGN-ECndss+PGRP+agonistic anti-TREM-1 antibody. Then, the expression of several genes (i.e., CCL20, IL-1β, IL-12p40, and IL-23β) that were highly induced (and inhibited with TREM-1 blocking antibody) from the 180 gene signature were selected, and their expression patterns assessed using RT-PCR and; or cytokine expression analysis.

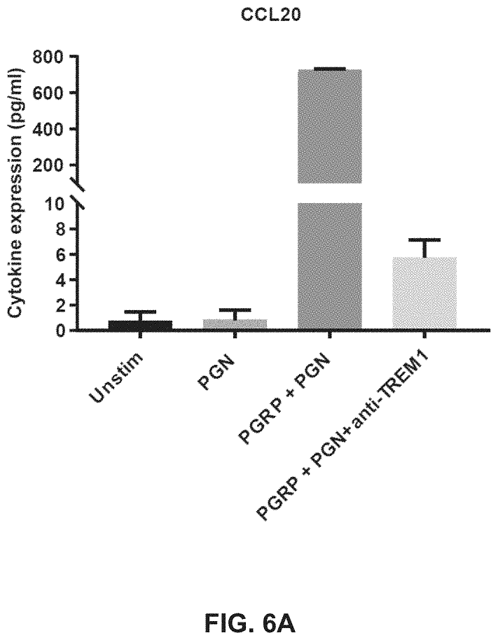

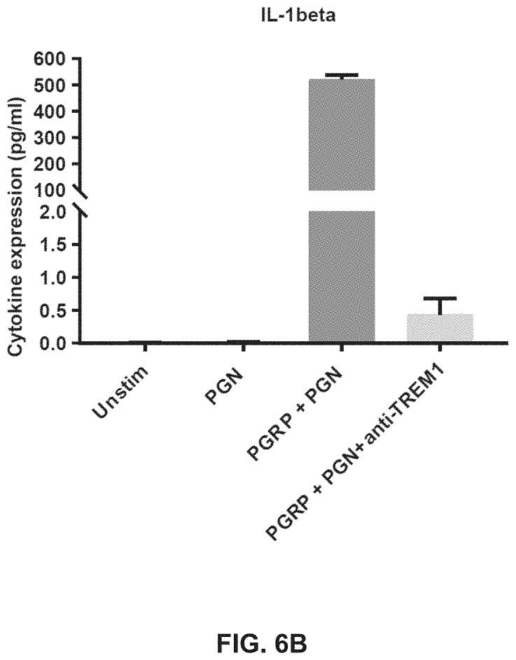

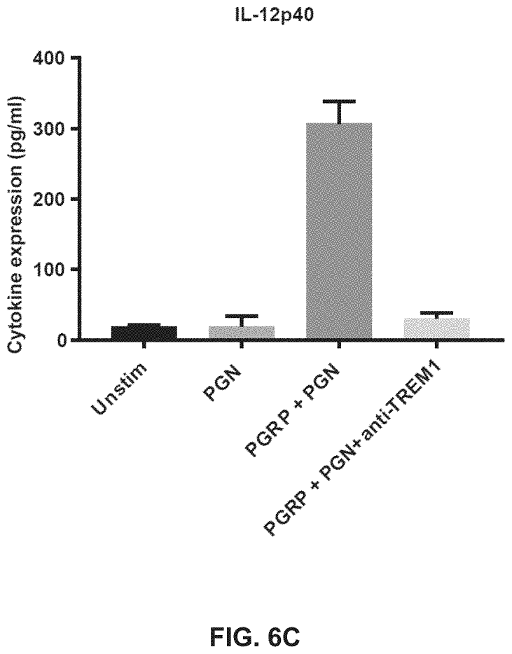

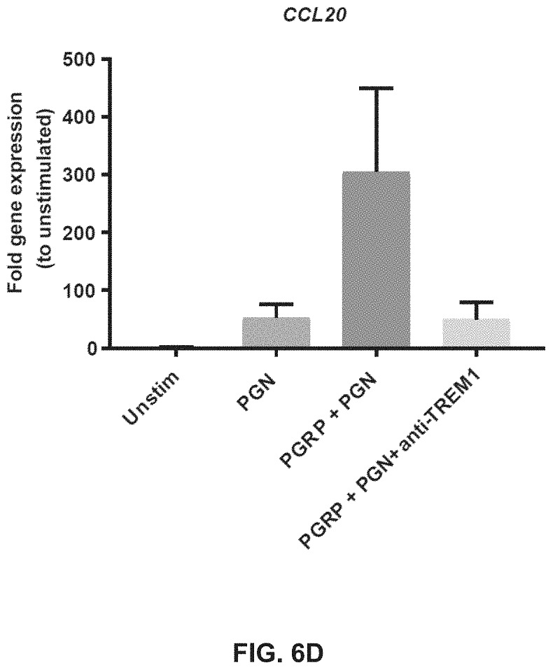

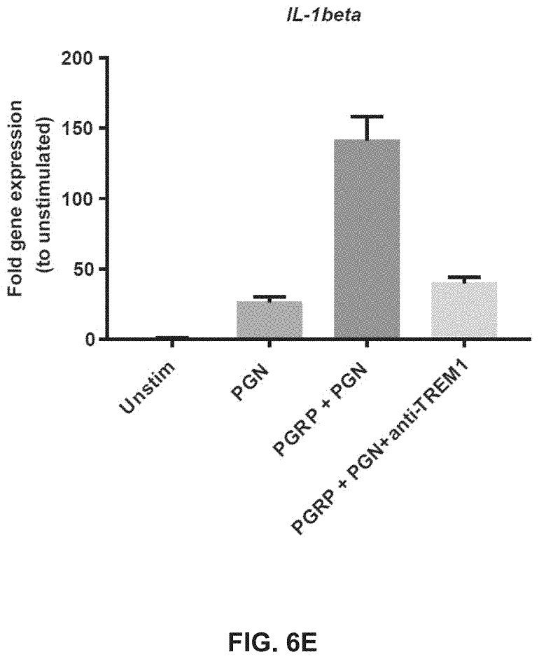

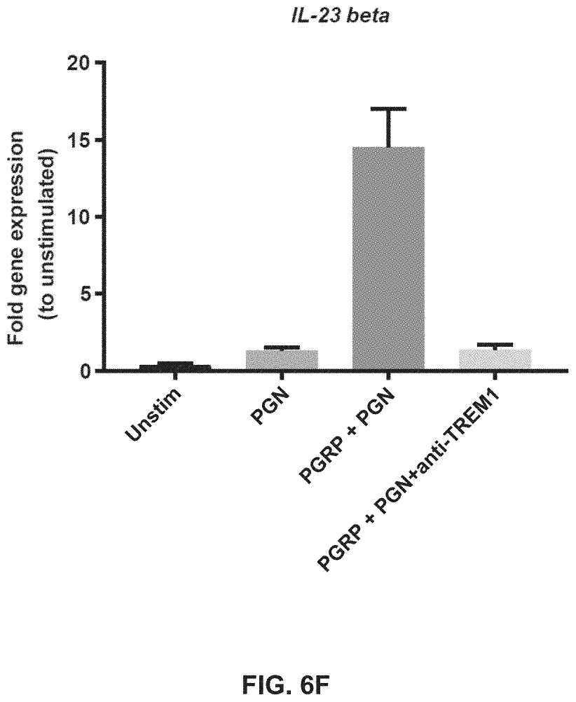

Similar to purified monocytes (see FIGS. 1A-1D), PGN-ECndss induced very little expression of CCL20, IL1β and IL12p40 (both at the protein and mRNA levels). See FIGS. 6A-6F. In contrast, PGLYRP1 and PGN-ECndss robustly induced both mRNA and protein expression of these cytokines and chemokine, which was blocked with TREM-1 blocking antibody (FIGS. 6A-6F). These results confirm that the findings in monocytes that TREM-1-induced changes can be blocked with anti-TREM-1 antibody can be also expanded to PBMCs and can potentially be used as a PD marker in clinical trials.

### Example 5: Distribution of TREM-1 Signature Score in IBD Biopsy Expression Profiling Datasets, at Baseline and after Treatment

The expression pattern of the TREM-1 gene module generated in the Examples above in colon biopsies from IBD patients was assessed. Gene expression in data from a phase 2 trial evaluating the efficacy of anti-IP10 antibody in UC patients (ClinicalTrials.gov identifier NCT00656890) were used as primary data source. At baseline (D1), matched lesional and non-lesional biopsies and whole blood samples from 78 UC patients were profiled using the Affymetrix U219 platform. As a secondary data source, a public dataset (GSE16879) was used to investigate gene expression before and after Infliximab (IFX) treatment in both CD and UC patients. This dataset contained gene expression profile (Affymetrix platform) from baseline and 4-6 weeks post-Infliximab treatment biopsies from 61 IBD patients (24 UC, 19 CD colon and 18 CD ileum) complete with clinical annotations and treatment response and from 12 non-IBD controls (6 colon, 6 ileum). For each patient, a ssGSEA score, a rank based score summarizing the collective expression enrichment for all genes in TREM-1 module, was calculated using the GSVA Bioconductor package in R.

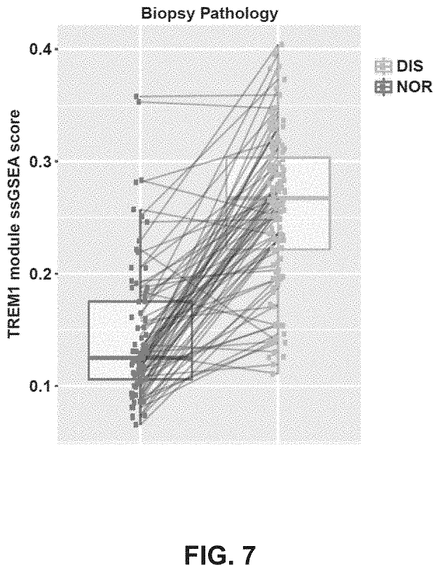

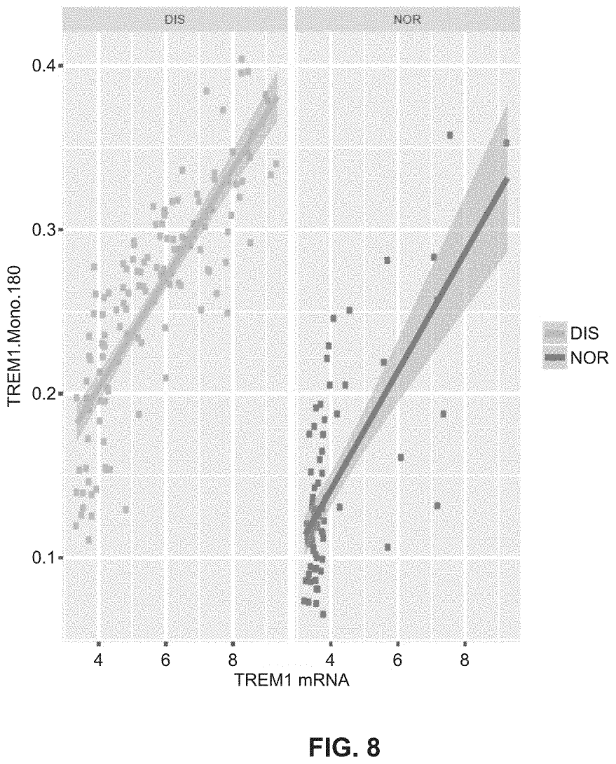

As shown in FIG. 7, in the anti-IP10 trial dataset, we found that TREM-1 module score was elevated in lesional compared to non-lesional biopsies at baseline (P value <0.001). The TREM-1 module score was also positively correlated with TREM-1 expression in UC lesional biopsies (Rho=0.85, Pvalue <0.001, FIG. 8), suggesting activation of TREM-1 at baseline in this patient population.

Next, how standard of care (SOC), e.g., oral steroids or use of anti-TNF agents, affects changes in TREM-1 module score in tissues was assessed. All patients from anti-IP10 trial with prior history of anti-TNF therapy were considered non-responders/inadequate responders (NR/IR). Patients without record of anti-TNF use were considered anti-TNF naïve.

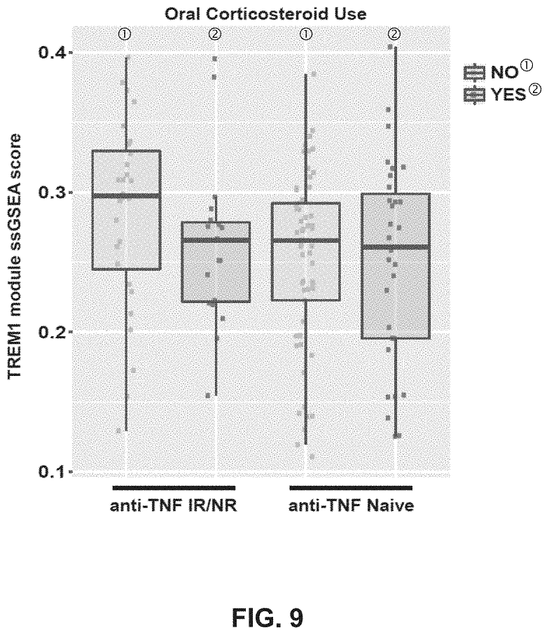

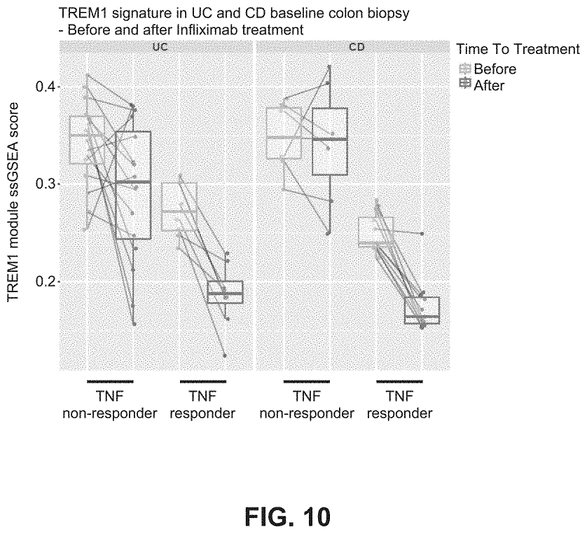

The TREM-1 gene signature score showed no statistical difference between anti-TNF naive and anti-TNF NR/IR IBD patients regardless of whether or not they used oral corticosteroids (FIG. 9). Analysis of the GSE16879 dataset revealed that TREM-1 module score remained elevated after treatment with IFX in those patients who were considered treatment non-responders, but decreased in TNF-responders (FIG. 10). Moreover, at baseline. IFX non-responders had significantly higher TREM-1 module scores compared to IFX responders (FIG. 10). These findings were applicable to UC and CD patients with colon involvement. Taken together, these data suggested that TREM-1 pathway was more active and remained elevated in the TNF non-responder population.

### Example 6: Application of TREM-1 Gene Module as Potential Blood Pharmacodynamics Biomarker Candidates

Since the genes in the TREM-1 gene module reflect the transcriptome change after TREM-1 pathway activation, whether such genes could be used as potential PD biomarker candidates was evaluated. To select blood-based PD marker candidates, genes in the TREM-1 gene module were filtered according to the following criteria: a) expression in UC baseline blood (mean log 2RMA >5); b) lower variation in UC baseline blood (IQR <0.7); c) expressed in extracellular space (GO functional annotation). The percentage inhibition after treatment with anti-TREM-1 was calculated by the magnitude of fold Change upon in PGLYRP1+PGN-ECndss divided by the fold change of PGLYRP1+PGN-ECndss vs PGN-ECndss stimulation. TREM-1 ligand stimulation was the log 2 fold change of PGLYRP1+PGN-ECndss stimulation, and these candidates were ranked in decreasing order. The top 20 blood PD candidates with mean and IQR of these mRNAs in IM129-005 UC baseline blood and internal healthy and UC blood are shown in Table 7. These genes will be evaluated in clinical trials in the future.

TABLE 7

| | | | IM129005 | IM129005 | | | |
|---|---|---|---|---|---|---|---|
| | | | Baseline | Baseline | | | |
| | | Log2FC | Blood | Blood | NHV | | |
| Gene | Inhibition | P + L vs P | Mean | IQR | Mean | UC Mean | Input Description |
| NAMPT | 86.14% | 3.4703 | 9.6758 | 0.801 | 10.6492 | 10.6395 | Nicotinamide |
| | | | | | | | phosphoribosyltrans |
| | | | | | | | ferase |
| DHRS9 | 94.62% | 3.2242 | 6.5785 | 1.301 | 6.557 | 7.7048 | Dehydrogenase/ |
| | | | | | | | reductase 9 |
| CDKN1A | 94.63% | 2.2972 | 6.9684 | 0.887 | 6.2774 | 6.367 | Cyclin dependent |
| | | | | | | | kinase inhibitor 1A |
| CD52 | 89.98% | 2.2767 | 9.2441 | 1.101 | 11.0883 | 11.1892 | CD52 molecule |
| MTMR11 | 94.83% | 2.583 | 4.5349 | 0.473 | 5.6913 | 5.4345 | Myotubularin |
| | | | | | | | related protein 11 |
| EHD1 | 92.46% | 3.3677 | 4.3785 | 0.426 | 3.7682 | 3.6972 | EH domain |
| | | | | | | | containing 1 |
| SLC27A3 | 95.98% | 2.743 | 5.5945 | 0.493 | 5.9187 | 5.8385 | Solute carrier |
| | | | | | | | family 27 member 3 |
| IL24 | 99.05% | 6.6649 | 8.0356 | 0.667 | 8.8407 | 8.717 | Interleukin 24 |
| PIM2 | 101.22% | 2.7146 | 8.7782 | 0.518 | 9.7709 | 9.819 | Pim-2 proto- |
| | | | | | | | oncogene, serine/ |
| | | | | | | | threonine kinase |
| CHI3L1 | 98.52% | 5.6198 | 10.6182 | 1.108 | 10.1509 | 10.016 | Chitinase 3 like 1 |
| GALNT6 | 95.82% | 2.0865 | 3.7005 | 0.374 | 3.9992 | 3.8948 | Polypeptide N- |
| | | | | | | | acetylgalactosamin- |
| | | | | | | | yltranserase |
| ACOT7 | 100.66% | 2.2271 | 4.2338 | 0.601 | 3.4673 | 3.5765 | Acyl-CoA |
| | | | | | | | thioesterase 7 |
| CISH | 93.27% | 3.283 | 7.424 | 0.741 | 7.9219 | 8.1807 | Cytokine inducible |
| | | | | | | | SH2 containing |
| | | | | | | | protein |
| FAM129A | 102.41% | 2.3118 | 9.6624 | 0.485 | 9.939 | 9.8118 | Family with |
| | | | | | | | sequence similarity |
| | | | | | | | 129 member A |
| PLK3 | 95.25% | 2.6663 | 4.4553 | 0.518 | 4.1541 | 4.0795 | Polo like kinase 3 |
| MFSD12 | 93.00% | 3.0698 | 4.8703 | 0.511 | 4.6705 | 4.4945 | Major facilitator |
| | | | | | | | superfamily |
| | | | | | | | domain containing |
| | | | | | | | 12 |
| STARD4 | 99.92% | 2.5623 | 3.144 | 0.21 | 3.8456 | 3.999 | StAR related lipid |
| | | | | | | | transfer domain |
| | | | | | | | containing 4 |
| CLEC12A | 103.28% | 2.9364 | 8.3115 | 1.705 | 9.3787 | 9.2685 | C-type lectin |
| | | | | | | | domain family 12 |
| | | | | | | | member A |
| CD55 | 80.39% | 2.3904 | 10.4693 | 0.541 | 10.183 | 10.215 | CD55 molecule |
| | | | | | | | (Cromer blood |
| | | | | | | | group) |
| IFNLR1 | 87.59% | 3.0729 | 6.071 | 0.97 | 7.2704 | 7.1378 | Interferon lambda |
| | | | | | | | receptor 1 |

### Example 7: Application of TREM-1 Module to Generate UC Specific TREM-1 Signature

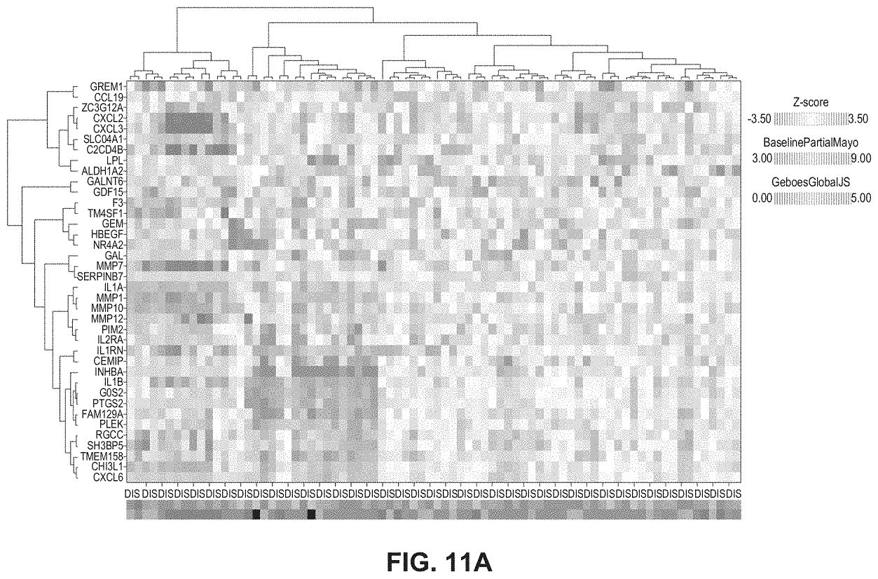

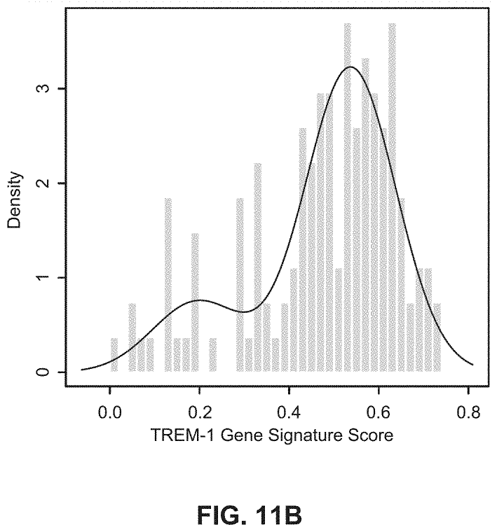

Based on TREM-1 gene module generated in the Examples above, a UC specific TREM-1 signature was generated using the following criteria: a) associated with TREM-1 pathway (genes from TREM-1 module); b) relevance in UC by expression in lesional biopsy (mean log 2RMA >4); c) relevance in UC by up-regulation in lesional biopsy (lesional vs non-lesional FDR <0.05); d) variably expressed in UC lesional biopsy samples (IQR >1). This filtering resulted in a signature with 38 genes, which represented potential patient stratification biomarker candidates. We then examined the expression pattern of these 38 genes in baseline UC colon biopsy from anti-IP10 trial (FIG. 11A).

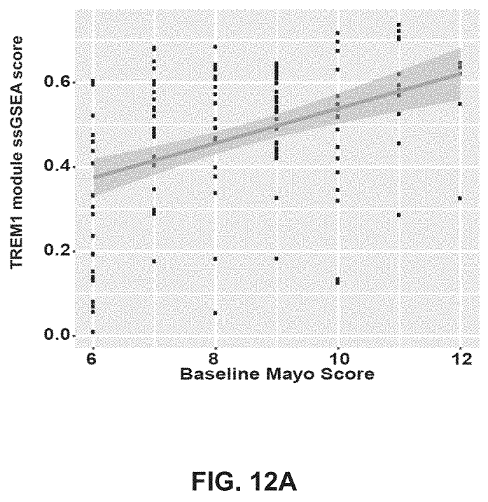

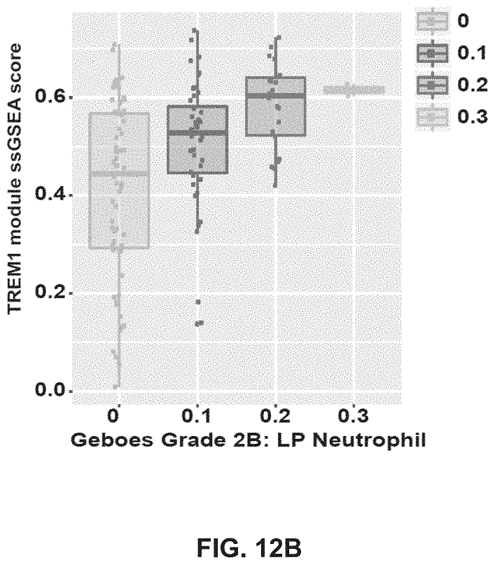

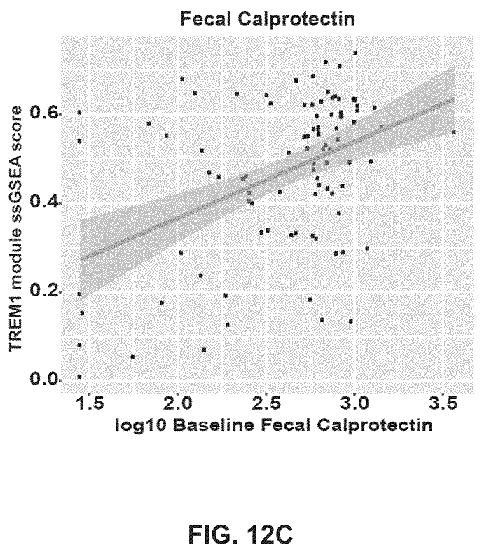

As shown in FIG. 11B, although there was substantial heterogeneity in expression of these genes across patients, two distinct clusters of patients were identified. This bi-modal distribution suggested the existence of a UC patient population with high TREM-1 module score (>0.33; FIG. 12B), which represented a majority of patient populations in this dataset. To further support the heterogeneity of TREM-1 module in UC patients, matched non-lesional tissue were used as a reference. Using the TREM-1 module score to predict lesional or non-lesional tissues, it was estimated that the score (0.25) achieving highest AUC could be an optimal value for separating patient population with positive or negative TREM-1 signature.

### Example 8: Application of TREM-1 Gene Signature to Identify Surrogate Biomarkers for Patient Stratification Via Correlation Analysis of Clinical Parameters

Assuming TREM-1 gene signature in lesional colon biopsy reflects pathway activation in IBD patients, the correlation of the TREM-1 gene signature in UC colon biopsy to other potential biomarkers was investigated. Since measuring TREM-1 gene signature requires lesional biopsy in disease tissue (e.g. colon) and either a dedicated gene panel or global RNASeq profiling, this investigation allowed for the identification of potential biomarkers as surrogate of TREM-1 pathway activation. These biomarkers could be easier to measure in the clinic.

A regression model was fitted to investigate the association of multiple clinical and biomarker factors with TREM-1 module score from patient stratification candidates (Table 8).

TABLE 8

| Predictive Factors | Effect Size | P Value |
|---|---|---|
| Baseline Mayo Score | 0.0315 | 0.00087 |
| Grade 2B LP Neutrophil | 0.732 | 0.00094 |
| Fecal Calprotectin | 7.92E−05 | 0.0152 |
| Duration of disease | −0.0033 | 0.142 |
| Imm Suppressant Use | 0.0349 | 0.256 |
| Grade 3 | 0.285 | 0.257 |
| Neutrophil Epithelium | | |
| Prior TNF Status | −0.0236 | 0.471 |
| Oral Corticosteroid | 0.00758 | 0.803 |

As shown in FIGS. 12A-12C, Baseline Mayo score, Grade 2B Lamina Propria Neutrophil Infiltration score (one component of Geboes score) and fecal calprotectin were among the factors significantly associated with TREM-1 gene signature in UC patients. The positive association signal other correcting with other factors suggested the existence of a UC patient population with both high fecal calprotectin (FC) and high TREM-1 score. In other words, a majority of UC patients with high FC levels can have high TREM-1 pathway activation. Since FC is easier to measure in the clinic, it may serve as a surrogate biomarker of TREM-1 gene signature.

TABLE 9
Exemplary Anti-TREM-1 Antibody Sequences

| SEQ | | |
|---|---|---|
| ID | Description | Sequences |
| 30 | 318-IgG1.3f | EVQLVESGGGLVQPGGSLKLSCAASGFTFSTYAMHWVRQASGKGLEWVGRIRTKSSNYA |
| | Heavy Chain | TYYAASVKGRFTTSRDDSKNTAYLQMNSLKTEDTAVYYCTRDMGIRRQFAYWGQGTLVT |
| | | VSSASTKGPSVFPLAPSSKSTSGGTAALGCLVKDYFPEPVTVSWNSGALTSGVHTFPAV |
| | | LQSSGLYSLSSVVTVPSSSLGTQTYTCNVNHKPSNTKVDKRVEPKSCDKTHTCPPCPAP |
| | | EAEGAPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 31 | 318-IgG1.1f | EVQLVESGGGLVQPGGSLKLSCAASGETFSTYAMHWVRQASGKGLEWVGRIRTKSSNYA |
| | Heavy Chain | TYYAASVKGRFTISRDDSKNTAYLQMNSLKTEDTAVYYCTRDMGIRRQFAYWGQGTLVT |
| | | VSSASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTCPPCPAP |
| | | EAEGAPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPSSIEKTISKAKGQPREPQVYT |
| | | LPPSREEMTKNQVSLTCLVKGFYPSDIAVEWESNGQPENNYKTTPPVLDSDGSFFLYSK |
| | | LTVDKSRWQQGNVFSCSVMHEALHNHYTQKSLSLSPG |
| 32 | 318-IgG1-Aba | EVQLVESGGGLVQPGGSLKLSCAASGFTFSTYAMHWVRQASGKGLEWVGRIRTKSSNYA |
| | Heavy Chain | TYYAASVKGRFTISRDDSKNTAYLQMNSLKTEDTAVYYCTRDMGIRRQFAYWGQGTLVT |
| | | VSSASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTSPPSPAP |
| | | ELLGGSSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 33 | 318-IgG4-Aba | EVQLVESGGGLVQPGGSLKLSCAASGFTFSTYAMHWVRQASGKGLEWVGRIRTKSSNYA |
| | Heavy Chain | TYYAASVKGRFTISRDDSKNTAYLQMNSLKTEDTAVYYCTRDMGIRRQFAYWGQGTLVT |
| | | VSSASTKGPSVFPLAPCSRSTSESTAALGCLVKDYFPEPVTVSWNSGALTSGVHTFPAV |
| | | LQSSGLYSLSSVVTVPSSSLGTKTYTCNVDHKPSNTKVDKRVEPKSCDKTHTSPPSPAP |
| | | ELLGGSSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 34 | 318-IgG1.3f, | DIVLTQSPDSLAVSLGERATINCRASQSVDTFDYSFLHWYQQKPGQPPKLLIYRASNLE |
| | IgG1.1f, IgG1- | SGVPDRFSGSGSGTDFTLTISSLQAEDVAVYYCQQSNQDPYTFGQGTKLEIKRTVAAPS |
| | Aba, IgG4-Aba | VFIFPPSDEQLKSGTASVVCLLNNFYPREAKVQWKVDNALQSGNSQESVTEQDSKDSTY |
| | Light Chain | SLSSTLTLSKADYEKHKVYACEVTHQGLSSPVTKSFNRGEC |
| 13 | mAb 0170 HC | EVQLVESGGGLVQPGGSLKLSCAASGFTFSTYAMHWVRQASGKGLEWVGRIRTKSSNYA |
| | | TYYAASVKGRFTISRDDSKNTAYLQMNSLKTEDTAVYYCTRDMGIRRQFAYWGQGTLVT |
| | | VSSASTKGPSVFPLAPCSRSTSESTAALGCLVKDYFPEPVTVSWNSGALTSGVHTFPAV |
| | | LQSSGLYSLSSVVTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGVEVHNAKTKPREE |
| | | QFNSTYRVVSVLTVLHQDWLNGKEYKCKVSNKGLPSSIEKTISKAKGQPREPQVYTLPP |
| | | SQEEMTKNQVSLTCLVKGFYPSDTAVEWESNGQPENNYKTTPPVLDSDGSFFLYSRLTV |
| | | DKSRWQEGNVFSCSVMHEALHNHYTQKSLSLSLGK |
| 14 | mAb 0170 LC | DIVLTQSPDSLAVSLGERATINCRASESVDTFDYSFLHWYQQKPGQPPKLLIYRASNLE |
| | | SGVPDRFSGSGSGTDFTLTISSLQAEDVAVYYCQQSNEDPYTFGQGTKLEIKRTVAAPS |
| | | VFTFPPSDEQLKSGTASVVCLLNNFYPREAKVQWKVDNALQSGNSQESVTEQDSKDSTY |
| | | SLSSTLTLSKADYEKHKVYACEVTHQGLSSPVTKSFNRGEC |
| 15 | mAb 0318 VH | EVQLVESGGGLVQPGGSLKLSCAASGFTFSTYAMHWVRQASGKGLEWVGRTRTKSSNYA |
| | | TYYAASVKGRFTISRDDSKNTAYLQMNSLKTEDTAVYYCTRDMGIRRQFAYWGQGTLVT |
| | | VSS |
| 23 | mAb 0318 VL | DIVLTQSPDSLAVSLGERATINCRASQSVDTFDYSFLHWYQQKPGQPPKLLIYRASNLE |
| | | SGVPDRFSGSGSGTDFTLTISSLQAEDVAVYYGQQSNQDPYTFGQGTKLEIK |
| 26 | mAb 0318 | EVQLVESGGGLVQPGGSLKLSCAASGFTFSTYAMHWVRQASGKGLEWVGRIRTKSSNYA |
| | mutant #1 VH | TYYAASVKGRFTISRDDSKNTAYLQMNSLKTEDTAVYYCTRDQGIRRQFAYWGQGTLVT |
| | | VSS |
| 27 | mAb 0318 | EVQLVESGGGLVQPGGSLKLSCAASGFTFSTYAMHWVRQASGKGLEWVGRIRTKSSNYA |
| | mutant #2 VH | TYYAASVKGRFTISRDDSKNTAYLQMNSLKTEDTAVYYCTRDLGIRRQFAYWGQGTLVT |
| | | VSS |
| 28 | mAb 0318 | EVQLVESGGGLVQPGGSLKLSCAASGFTFSTYAQHWVRQASGKGLEWVGRIRTKSSNYA |
| | mutant #3 VH | TYYAASVKGRFTISRDDSKNTAYLQMNSLKTEDTAVYYCTRDMGIRRQFAYWGQGTLVT |
| 29 | mAb 0318 | EVQLVESGGGLVQPGGSLKLSCAASGFTFSTYALHWVRQASGKGLEWVGRIRTKSSNYA |
| | mutant #4 VH | TYYAASVKGRETISRDDSKNTAYLQMNSLKTEDTAVYYCTRDMGIRRQFAYWGQGTLVT |
| 53 | P1-047248 VH | QVQLQESGPGLVKPSETLSLTCTVSGGSISSSYWSWVRQPPGKGLEWIGYTHYSGISNY |
| | | NPSLKSRVTISVDTSKNQFSLKLSSVTAADTAVYYCAREGYDILTGYEYYGMDVWGQGT |
| | | TVTVSS |
| 54 | P1-047248 VL | EIVLTQSPGTLSLSPGERATLSCRASQSVSSSYLAWYQQKPGQAPRLLIYGASSRATGI |
| | | PDRFSGSGSGTDFTLTISRLEPEDFAVYYCQQYGSSPTFGGGTKVEIK |
| 55 | P1-047246 VH | QVQLQESGPGLVKPSETLSLTCTVSGGSITNYYWTWIRQPPGKGLEWIGYIYDSGYTNY |
| | | NPSLKSRVTLSIDTSKNQFSLKLSSVTAADTAVYYCARGVLWFGELLPLLDYWGQGTLV |
| | | TVSS |
| 56 | P1-047246 VL | AIQLTQSPSSLSASVGDRVTITCRASQGISSALAWYQQKPGKAPKLLIYDASSLESGVP |
| | | SRFSGSGSGTDFTLTISSLQPEDFATYYCQQFNSYPYTFGQGTKLEIK |
| 55 | P1-047247 VH | QVQLQESGPGLVKPSETLSLTCTVSGGSITNYYWTWIRQPPGKGLEWIGYIYDSGYTNY |
| | | NPSLKSRVTLSIDTSKNQFSLKLSSVTAADTAVYYCARGVLWFGELLPLLDYWGQGTLV |
| | | TVSS |
| 55 | P1-047247 VL | EIVLTQSPGTLSLSPGERATLSCRASQSVSSSYLAWYQQKPGQAPRLLIYGASSRATGI |
| | | PERFSGSGSGTDFTLTISRLEPEDFAVYYCQQYGSSPLTFGGGTKVEIK |
| 58 | P1-047334 VH | QVQLVQSGAEVKRPGSSVKVSCKASGGTFSSSAISWVRQAPGQGLEWMGGIIPIFGTTN |
| | | GAQKFQGRVTITADESTSTAYMELSSLRSEDTAVYYCAAMVRGNYFYFYGMDVWGQGTT |
| | | VTVSS |
| 59 | P1-047334 VL | AIQLTQSPSSLSASVGDRVTITCRASQGISSALAWYQQKPGKAPKLLIYAASSLQSGVP |
| | | SRFSGSGSGTDFTLTISSLQPEDFATYYCQQYNSYPLTFGGGTKVEIK |
| 60 | P1-047239 VH | QVQLVESGGGVVQPGRSLRLSCAATEFTFSNYGMHWVRQAPGKGLEWVAVIWYDGSNKY |
| | | YADSVKGRFTTSRDNSKNTLYLQLNSLSAEDSAVYYCARDGRHYYGSTSYFGMDVWGQG |
| | | TTVTVSS |
| 56 | P1-047239 VL | AIQLTQSPSSLSASVGDRVTITCRASQGISSALAWYQQKPGKAPKLLIYDASSLESGVP |
| | | SRFSGSGSGTDFTLTISSLQPEDFATYYCQQFNSYPYTFGQGTKLEIK |
| 153 | P1-047323 VH | QVQLVQSGAEVKKPGSSVKVSCKASGGTFINSEAINWVRQAPGQGLEWMGGIIPIFDIT |
| | | NYAQKFQGRVTITADESMSTAYMELSSLRSEDTAVYYCAKTYYDILTYHYHYGMDVWGQ |
| | | GTTVTVSS |
| 154 | P1-047323 VL | AIQLTQSPSSLSASVGDRVTITCRASQGISSALAWYQQKPGKAPKLLIYDASSLESGVP |
| | | SRFSGSGSGTDFTLTISSLQPEDFATYYCQQFNSYPITFGQGTRLEIK |
| 153 | PL047328 VH | QVQLVQSGAEVKKPGSSVKVSCKASGGTFTNSEAINWVRQAPGQGLEWMGGIIPIFDIT |
| | | NYAQKFQGRVTITADESMSTAYMELSSLRSEDTAVYYCAKTYYDILTYHYHYGMDVWGQ |
| | | GTTVTVSS |
| 155 | P1-047328 VL | AIQLTQSPSSLSASVGDRVTITCRASQGISSALAWYQQKPGKAPKLLIYDASSLESGVP |
| | | SRFSGSGSGTDFTLTISSLQPEDFATYYCQQYNSYPITFGQGTRLEIK |
| 82 | P1-047305 VH | QVQLVQSGAEVKKPGSSVKVSCKTSGGTFSSSAVSWVRQAPGQGLEWMGGITPIFGTAD |
| | | YAQKFQGRVTITADASTSTGYMELSSLRSEDTAVYYCAFTPRYRGSSHHYYYALGVWGQ |
| | | GTTVTVSS |
| 83 | P1-047305 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPYTFGQGTKLEIK |
| 84 | P1-047309 VH | QVQLVQSGAEVKKPGSSVKVSCNPSGGTFSTYAISWVRQAPGQGLEWMGGINPIFGTAN |
| | | YAQKFQGRVTTTADESTSPGYLELSSLRSEDTAVYYCARGGAVGFAYWGQGTLVTVSS |
| 85 | P1-047309 VL | DIQMTQSPSSVSASVGDRVTITCRASQGISSWLAWYQHKPGKAPKLLTYAASSLQSGVP |
| | | SRFSGSGSGTDFTLTISSLQPEDFATYYCQQANSFPFTFGGGTKVEIK |
| 86 | P1-047313 VH | QVQLVQSGAEVKRPGSSVKVSCKASGGTFSIYVISWVRQAPGQGLEWMGGIIPLFGTPN |
| | | YAQRFQDRVTITADESTRTAYMELSSLRSEDTAVYYCARGHGPGSSHYSYYGLDVWGQG |
| | | TTVTVSS |
| 87 | P1-047313 VL | DIQMTQSPTSLSASVGDRVTITCRASQGISSWLAWYQHKPGKAPKLLIYAASSLQSGVP |
| | | SRFSGSGSGTDFTLTISSLQPEDFATYYCQQYNSYPWTFGQGTKVEIK |
| 88 | P1-047307 VH | QVQLVQSGAEVKRPGSSVKVSCKASGGTFSIYVISWVRQAPGQGLEWMGGIIPLHGTPN |
| | | YAQQFQDRVTITADESTRTAYMELNSLKSEDTAVYYCARGHGPGSSHYSYYGLDVWGQG |
| | | TTVTVSS |
| 89 | P1-047307 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPLTFGGGTKVEIK |
| 88 | P1-047312 VH | QVQLVQSGAEVKRPGSSVKVSCKASGGTFSIYVISWVRQAPGQGLEWMGGIIPLFGTPN |
| | | YAQQFQDRVTITADESTRTAYMELNSLKSEDTAVYYCARGHGPGSSHYSYYGLDVWGQG |
| | | TVTVSS |
| 90 | P1-047312 VL | DIQMTQSPTSLSASVGDRVTITCRASQGISSWLAWYQHKPGKAPKLLIYAASSLQSGVP |
| | | SRFSGSGSGTDFTLTISSLQPEDFATYYCQQYNSYPYTFGQGTKLEIK |
| 88 | P1-047314 VH | QVQLVQSGAEVKRPGSSVKVSCKASGGTFSIYVISWVRQAPGQGLEWMGGIIPLFGTPN |
| | | YAQQFQDRVTITADESTRTAYMELNSLKSEDTAVYYCARGHGPGSSHYSYYGLDVWGQG |
| | | TTVTVSS |
| 83 | P1-047314 VL | DIQMTQSPTSLSASVGDRVTITCRASQGISSWLAWYQQKPEKAPKSLIYAASSLQSGVP |
| | | SRFSGSGSGTDFTLTTSSLQPEDFATYYCQQYNSYPYTFGQGTKLETK |
| 91 | P1-047318 VH | QVQLVQSGAEVKRPGSSVKVSCKASGGTFSIYVISWVRQAPGQGLEWMGGIIPLFGTPN |
| | | YAQQFQDRVTITADESTRTAYMELSSLRSEDTAVYYCARGHGPGSSHYSYYGLDVWGQG |
| | | TTVTVSS |
| 90 | P1-047318 VL | DIQMTQSPTSLSASVGDRVTITCRASQGISSWLAWYQHKPGKAPKLLIYAASSLQSGVP |
| | | SRFSGSGSGTDFTLTISSLQPEDFATYYCQQYNSYPYTFGQGTKLEIK |
| 88 | P1-047320 VH | QVQLVQSGAEVKRPGSSVKVSCKASGGTFSIYVISWVRQAPGQGLEWMGGIIPLHGTPN |
| | | YAQQFQDRVTITADESTRTAYMELNSLKSEDTAVYYCARGHGPGSSHYSYYGLDVWGQG |
| | | TTVTVSS |
| 92 | P1-047320 VL | DIQMTQSPTSLSASVGDRVTITCRASQGISSWLAWYQHKPGKAPKSLTYAASSLQSGVP |
| | | SRFSGSGSGTDFTLTISSLQPEDFATYYCQQYNSYPYTFGQGTKLEIK |
| 93 | P1-047311VH | QVQLVQSGAEVKKPGSSVKVSCKASGGTFSIYVISWVRQAPGQGLEWMGGIIPLFGTAN |
| | | YAQQFQDRVTITADESTRTAYMELNSLKSEDTAVYYCARGHGPGSSHYSYYGLDVWGQG |
| | | TVTVSS |
| 94 | P1-047311 VL | DIQMTQSPTSLSASVGDRVTITCRASQGISSWLAWYQQKPGKAPKSLIYAASSLQSGVP |
| | | SRFSGSGSGTDFTLTISSLQPEDFATYYCQQYNSYPLTFGGGTKVEIK |
| 95 | P1-047294 VH | QVQLVQSGAEVKKPGSSVKVSCKASGGTFNRHAISWVRQAPGQGLEWMGGIIPLFGTAN |
| | | YAQEFQGRVTIAADEPTSTTYMELRSLRSEDTAVYYCASSYFYGSGSSNYYYYGLDVWG |
| | | QGTTVTVSS |
| 96 | P1-047294 VL | DIQMTQSPTSLSASVGDRVTITCRASQGISSWLAWYQQKPEKAPKSLIYAASSLQSGVP |
| | | SRFSGGGSGTDFTLTISSLQPEDFATYYCQQYNSYPLTFGGGTKVEIK |
| 97 | P1-047290 VH | QVQLVQSGAEVKKPGSSVKVSCKASGGTFNRHAISWVRQAPGQGLEWMGGIIPLFGTAN |
| | | YAQKFQGRVTIAADEPTSTTYMELRSLRSEDTAVYYCASSYFYGSGSSNYYYYGLDVWG |
| | | QGTTVTVSS |
| 83 | P1-047290 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPYTFGQGTKLEIK |
| 97 | P1-047291 VH | QVQLVQSGAEVKKPGSSVKVSCKASGGTFNRHAISWVRQAPGQGLEWMGGIIPLFGTAN |
| | | YAQKFQGRVTIAADEPTSTTYMELRSLRSEDTAVYYCASSYFYGSGSSNYYYYGLDVWG |
| | | QGTTVTVSS |
| 98 | P1-047291 VL | DIQMTQSPTSLSASVGDRVTITCRASQGISSWLAWYQQKPEKAPKSLTYAASSLQSGVP |
| | | SRFSGSGSGTDFTLTISSLQPDDFATYYCQQYNSYPYTFGQGTKLEIK |
| 97 | P1-047296 VH | QVQLVQSGAEVKKPGSSVKVSCKASGGTFNRHAISWVRQAPGQGLEWMGGIIPLFGTAN |
| | | YAQKFQGRVTIAADEPTSTTYMELRSLRSEDTAVYYCASSYFYGSGSSNYYYYGLDVWG |
| | | QGTTVTVSS |
| 99 | P1-047296 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPITFGQGTRLEIK |
| 97 | P1-047297 VH | QVQLVQSGAEVKKPGSSVKVSCKASGGTFNRHAISWVRQAPGQGLEWMGGIIPLFGTAN |
| | | YAQKFQGRVTIAADEPTSTTYMELRSLRSEDTAVYYCASSYFYGSGSSNYYYYGLDVWG |
| | | QGTTVTVSS |
| 100 | P1-047297 VL | DIQMTQSPTSLSASVGDRVTITCRASQGISSWLAWYQHKPGKAPKSLIYAASSLQSGVP |
| | | SRFSGSGSGTDFTLTISSLQPEDFATYYCQQYNSYPLTFGGGTKVEIK |
| 97 | P1-047300 VH | QVQLVQSGAEVKKPGSSVKVSCKASGGTFNRHAISWVRQAPGQGLEWMGGIIPLFGTAN |
| | | YAQKFQGRVTIAADEPTSTTYMELRSLRSEDTAVYYCASSYFYGSGSSNYYYYGLDVWG |
| | | QGTTVTVSS |
| 101 | P1-047300 VL | DIQMTQSPTSLSASVGDRVTITCRASQGISSWLAWYQQKPEKAPKSLIHAASSLQSGVP |
| | | SRFSGSGSGTDFTLTISSLQPEDFATYYCQQYNSYPLTFGGGTKVEIK |
| 97 | P1-047302 VH | QVQLVQSGAEVKKPGSSVKVSCKASGGTFNRHAISWVRQAPGQGLEWMGGIIPLFGTAN |
| | | YAQKFQGRVTIAADEPTSTTYMELRSLRSEDTAVYYCASSYFYGSGSSNYYYYGLDVWG |
| | | QGTTVTVSS |
| 89 | P1-047302 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPLTFGGGTKVEIK |
| 102 | P1-047308 VH | QVQLVQSGAEVKRPGSSVKVSCKASGGTFSIYVISWVRQAPGQGLEWMGGIIPLFGTAN |
| | | YAQKFQGRVTTTADESTNTAYMELSSLRSEDTAVYYCARGHGPGSSHYSYYGLDVWGQG |
| | | TTVTVSS |
| 83 | P1-047308 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPYTFGQGTKLEIK |
| 102 | P1-047319 VH | QVQLVQSGAEVKRPGSSVKVSCKASGGTFSIYVISWVRQAPGQGLEWMGGIIPLFGTAN |
| | | YAQKFQGRVTITADESTNTAYMELSSLRSEDTAVYYCARGHGPGSSHYSYYGLDVWGQG |
| | | TTVTVSS |
| 92 | P1-047319 VL | DIQMTQSPTSLSASVGDRVTITCRASQGISSWLAWYQHKPGKAPKSLIYAASSLQSGVP |
| | | SRFSGSGSGTDFTLTISSLQPEDFATYYCQQYNSYPYTFGQGTKLEIK |
| 103 | P1-047292 VH | QVQLVQSGAEVKKPGSSVKVSCKASGGTFSSSAISWVRQAPGQGLEWMGGIIPIFSTGN |
| | | YAQKFQGRVTITADESTNTAYMDLSSLRSEDTAVYYCARSTRVRGVSHYYYYGLDVWGQ |
| | | GTTVTVSS |
| 83 | P1-047292 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPYTFGQGTKLEIK |
| 104 | P1-047322 VH | QVQLVQSGAEVKKPGSSVKVSCKSSGGTFSSYAFTWVRQAPGQGLEWMGGIIPIFRTAN |
| | | YAQKFQGRVTITADESTSTAYMELSSLRSEDTAVYYCASSHFSGSGSSHYYYYGMHVWG |
| | | QGTTVTVSS |
| 83 | P1-047322 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPYTFGQGTKLEIK |
| 105 | P1-047310 VH | QVQLVQSGAEVKKPGSSVKVSCKASGGTFSRYAISWVRQAPGQGLEWMGGIIPIFGTSN |
| | | YAQKFQGRVTTKADESTSTAYMELSSLRSEDTAVYYCARGGNSWTTSLYYYGMDVWGQG |
| | | TTVTVSS |
| 106 | P 1-047310 VL | DIQMTQSPTSLSASVGDRVTITCRASQGISSWLAWYQQKPEKAPKSLIYAASSLQSGVP |
| | | SRFSGSGSGTDYTLTISSLQPEDFATYYCQQYNSYPLTFGGGTKVEIK |
| 107 | P1-047299 VH | QVQLVQSGAEVKKPGSSVKVSCKASGGTFNRYAFSWVRQAPGQGLEWMGGIIPIFGTPN |
| | | YAQKFQGRVTITADESTSTAYMELSSLISEDTAVYYCASSHFYGSGSSHFYYYGMHVWG |
| | | QGTTVTVSS |
| 108 | P1-047299 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPWTFGQGTKVEIK |
| 109 | P1-047301 VH | QVQLVQSGAEVKKPGSSVKVSCKASGGTFNRYAFSWVRQAPGQGLEWMGGIIPIFGTPN |
| | | YAQKFQGRVTITADESTSTAYMELSSLISEDTAVYYCASSHFYGSGSSNYYYYGLDVWG |
| | | QGTTVTVSS |
| 83 | P1-047301 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPYTFGQGTKLEIK |
| 110 | P1-047289 VH | QVQLVQSGAEVKKPGSSVKVSCKASGGTFSSSAISWVRQAPGQGLEWMGGIIPIFGTAD |
| | | SAQKFQGRVTITADESTSTAYMELNSLRSEDTAVYYCAFTPRYRGSSHHYFYALGVWGQ |
| | | GTTVTVSS |
| 111 | P1-047289 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQFNSYPLTFGGGTKVEIK |
| 112 | P1-047306 VH | QVQLVQSGAEVKKPGSSVKVSCKASGGTFSSSAISWVRQAPGQGLEWMGGIIPIFGTAN |
| | | YAQKFQGRVTTTADESTSTAYMELSSLRSEDTAVYYCARASQSRSSNYYYYGLDVWGQG |
| | | TTVTVSS |
| 89 | P1-047306 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPLTFGGGTKVEIK |
| 156 | P1-047263 VH | QVQLVQSGAEVKKPGASVKVSCKASGYTFPTYDINWVRQATGQGLEWMGWVNPNSGNTG |
| | | YAQKFQDRVTMTRNTSISTAYMELSSLRSEDTAVYYCASDGLNMVRGVHNYYGMDVWGQ |
| | | GTTVTVSS |
| 157 | P1-047263 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPPTFGQGTKLEIK |
| 156 | P1-047265 VH | QVQLVQSGAEVKKPGASVKVSCKASGYTFPTYDINWVRQATGQGLEWMGWVNPNSGNTG |
| | | YAQKFQDRVTMTRNTSISTAYMELSSLRSEDTAVYYCASDGLNMVRGVHNYYGMDVWGQ |
| | | GTTVTVSS |
| 83 | P1-047265 VL | DIQMTQSPTSLSASVGDRVTITCRASQGISSWLAWYQQKPEKAPKSLIYAASSLQSGVP |
| | | SRFSGSGSGTDFTLTISSLQPEDFATYYCQQYNSYPYTFGQGTKLEIK |
| 88 | P1-047317 VH | QVQLVQSGAEVKRPGSSVKVSCKASGGTFSIYVISWVRQAPGQGLEWMGGIIPLFGTPN |
| | | YAQQFQDRVTITADESTRTAYMELNSLKSEDTAVYYCARGHGPGSSHYSYYGLDVWGQG |
| | | TVTVSS |
| 158 | P1-047317 VL | EIVLTQSPGTLSLSPGERATLSCRASQSVSSYLAWYQQKPGQAPRLLIYGASSRATGIP |
| | | DRFSGSGSGTDPTLTISRLEPEDFAVYYCQQYGSSPLTFGGGTKVEIK |

1. A method of treating a disease or disorder in a subject in need thereof, comprising administering a therapeutically effective dose of an antagonistic anti-TREM-1 antibody to the subject, wherein the subject exhibits an increase in an expression level of a TREM-1 associated gene,
wherein the TREM-1 associated gene comprises Nicotinamide phosphoribosyltransferase (NAMPT); dehydrogenase/reductase 9 (DHRS9); cyclin dependent kinase inhibitor 1A (CDKN1A); CD52 molecule (CD52); Myotubularin related protein 11 (MTMR11); EH domain containing 1 (EHD1); Solute carrier family 27 member 3 (SLC27A3); Interleukin 24 (IL24); Pim-2 proto-oncogene, serine/threonine kinase (PIM2); chitinase 3 like 1 (CHI3L1); Polypeptide N-acetylgalactosaminyltransferase 6 (GALNT6); Acyl-CoA thioesterase 7 (ACOT7); cytokine inducible SH2 containing protein (CISH); family with sequence similarity 129 member A (FAM129A); polo like kinase 3 (PLK3); major facilitator superfamily domain containing 12 (MFSD12); StAR related lipid transfer domain containing 4 (STARD4); C-type lectin domain family 12 member A (CLEC12A); CD55 molecule (Cromer blood group) (CD55); Interferon lambda receptor 1 (IFNLR1), or combinations thereof.

2. The method of claim 1, wherein the subject was previously treated with a standard of care treatment for the disease or disorder and did not respond to the treatment, preferably wherein the standard of care treatment comprises an anti-TNF-α antibody, preferably wherein the anti-TNF-α antibody comprises infliximab (REMICADE®), certolizumab pegol (CIMZIA®), etanercept (ENBREL®), adalimumab (HUMIRA®), golimumab (SIMPONI®), or combinations thereof.

3. A method of identifying a subject suffering from a disease or disorder suitable for a treatment with an antagonistic anti-TREM-1 antibody, comprising
measuring an expression level of a TREM-1 associated gene in a sample of the subject,
wherein the TREM-1 associated gene comprises Nicotinamide phosphoribosyltransferase (NAMPT); dehydrogenase/reductase 9 (DHRS9); cyclin dependent kinase inhibitor 1A (CDKN1A); CD52 molecule (CD52); Myotubularin related protein 11 (MTMR11); EH domain containing 1 (EHD1); Solute carrier family 27 member 3 (SLC27A3); Interleukin 24 (IL24); Pim-2 proto-oncogene, serine/threonine kinase (PIM2); chitinase 3 like 1 (CHI3L1); Polypeptide N-acetylgalactosaminyltransferase 6 (GALNT6); Acyl-CoA thioesterase 7 (ACOT7); cytokine inducible SH2 containing protein (CISH); family with sequence similarity 129 member A (FAM129A); polo like kinase 3 (PLK3); major facilitator superfamily domain containing 12 (MFSD12); StAR related lipid transfer domain containing 4 (STARD4); C-type lectin domain family 12 member A (CLEC12A); CD55 molecule (Cromer blood group) (CD55); Interferon lambda receptor 1 (IFNLR1), or combinations thereof.

4. The method of claim 3, further comprising administering a therapeutically effective dose of the antagonistic anti-TREM-1 antibody to a subject who exhibits an increase in the expression level of the TREM-1 associated gene compared to a reference, wherein the reference comprises a subject not suffering from the disease or disorder (e.g., healthy subject).

5. A method of identifying a non-responder to a standard of care treatment for a disease or disorder, comprising
measuring an expression level of a TREM-1 associated gene in a sample of a subject who has received the standard of care treatment, preferably wherein the standard of care treatment comprises an anti-TNF-α antibody (e.g., INFLIXIMAB®),
wherein the subject exhibits an increase in the expression level of the TREM-1 associated gene and
wherein the TREM-1 associated gene comprises Nicotinamide phosphoribosyltransferase (NAMPT); dehydrogenase/reductase 9 (DHRS9); cyclin dependent kinase inhibitor 1A (CDKN1A); CD52 molecule (CD52); Myotubularin related protein 11 (MTMR11); EH domain containing 1 (EHD1); Solute carrier family 27 member 3 (SLC27A3); Interleukin 24 (IL24); Pim-2 proto-oncogene, serine/threonine kinase (PIM2); chitinase 3 like 1 (CHI3L1); Polypeptide N-acetylgalactosaminyltransferase 6 (GALNT6); Acyl-CoA thioesterase 7 (ACOT7); cytokine inducible SH2 containing protein (CISH); family with sequence similarity 129 member A (FAM129A); polo like kinase 3 (PLK3); major facilitator superfamily domain containing 12 (MFSD12); StAR related lipid transfer domain containing 4 (STARD4); C-type lectin domain family 12 member A (CLEC12A); CD55 molecule (Cromer blood group) (CD55); Interferon lambda receptor 1 (IFNLR1), or combinations thereof.

6. The method of claim 5, further comprising administering an additional therapeutic agent to a subject who has been identified as a non-responder to the standard of care treatment, preferably wherein the additional therapeutic agent comprises an antagonistic anti-TREM-1 antibody.

7. A method of determining efficacy of an antagonistic anti-TREM-1 antibody in treating a disease or disorder in a subject in need thereof, comprising administering the antagonistic anti-TREM-1 antibody to the subject and measuring an expression level of a TREM-1 associated gene in a sample of the subject, wherein the subject exhibits a decrease in the expression level of the TREM-1 associated gene after the administration,
wherein the TREM-1 associated gene comprises Nicotinamide phosphoribosyltransferase (NAMPT); dehydrogenase/reductase 9 (DHRS9); cyclin dependent kinase inhibitor 1A (CDKN1A); CD52 molecule (CD52); Myotubularin related protein 11 (MTMR11); EH domain containing 1 (EHD1); Solute carrier family 27 member 3 (SLC27A3); Interleukin 24 (IL24); Pim-2 proto-oncogene, serine/threonine kinase (PIM2); chitinase 3 like 1 (CHI3L1); Polypeptide N-acetylgalactosaminyltransferase 6 (GALNT6); Acyl-CoA thioesterase 7 (ACOT7); cytokine inducible SH2 containing protein (CISH); family with sequence similarity 129 member A (FAM129A); polo like kinase 3 (PLK3); major facilitator superfamily domain containing 12 (MFSD12); StAR related lipid transfer domain containing 4 (STARD4); C-type lectin domain family 12 member A (CLEC12A); CD55 molecule (Cromer blood group) (CD55); Interferon lambda receptor 1 (IFNLR1), or combinations thereof, optionally wherein the subject is continued with the antagonistic anti-TREM-1 antibody treatment.

8. The method of claim 1, wherein the subject also exhibits one or more of an increased Baseline Mayo score, an increased Grade 2B Lamina Propria Neutrophil Infiltration score, and an increased fecal calprotectin level, prior to the administration of the antagonistic anti-TREM-1 antibody, wherein
(a) the subject exhibits an increased Baseline Mayo score by at least about 5%, at least about 10%, at least about 20%, at least about 30%, at least about 40%, at least about 50%, at least about 60%, at least about 70%, at least about 80%, at least about 90% or more compared to the reference;
(b) the subject exhibits a Baseline Mayo score greater than about 6, 7, 8, 9, 10, 11, or 12 prior to the administration;
(c) the subject exhibits an increased Grade 2B Lamina Propria Neutrophil Infiltration score by at least about 5%, at least about 10%, at least about 20%, at least about 30%, at least about 40%, at least about 50%, at least about 60%, at least about 70%, at least about 80%, at least about 90% or more compared to the reference;
(d) the subject exhibits a Grade 2B Lamina Propria Neutrophil Infiltration score greater than about 0, about 0.1, about 0.2, or about 0.3;
(e) the subject exhibits an increased fecal calprotectin level by at least about 5%, at least about 10%, at least about 20%, at least about 30%, at least about 40%, at least about 50%, at least about 60%, at least about 70%, at least about 80%, at least about 90% or more compared to the reference; and/or
(f) the subject exhibits a fecal calprotectin level (μg/g of feces) greater than about 1.5 log 10, greater than about 2.0 log 10, greater than about 2.5 log 10, greater than about 3.0 log 10, or greater than about 3.5 log 10.

9. The method of claim 1, further comprising measuring one or more scores of a Baseline Mayo score, a Grade 2B Lamina Propria Neutrophil Infiltration score, and a fecal calprotectin level, prior to, concurrently, or after the measuring the expression level of the TREM-1 associated gene and/or administering the antagonistic anti-TREM-1 antibody.

10. The method of claim 1, wherein the administering the antagonistic anti-TREM-1 antibody reduces the expression of the TREM-1 associated gene, preferably wherein the administering the antagonistic anti-TREM-1 antibody also reduces a Baseline Mayo score, Grade 2B Lamina Propria Neutrophil Infiltration score, and/or fecal calprotectin level of the subject, wherein
(a) the Baseline Mayo score is decreased by at least about 5%, at least about 10%, at least about 20%, at least about 30%, at least about 40%, at least about 50%, at least about 60%, at least about 70%, at least about 80%, at least about 90% or more;
(b) the Grade 2B Lamina Propria Neutrophil Infiltration score is decreased by at least about 5%, at least about 10%, at least about 20%, at least about 30%, at least about 40%, at least about 50%, at least about 60%, at least about 70%, at least about 80%, at least about 90% or more; and/or
(c) the fecal calprotectin level is decreased by at least about 5%, at least about 10%, at least about 20%, at least about 30%, at least about 40%, at least about 50%, at least about 60%, at least about 70%, at least about 80%, at least about 90% or more.

11. The method of claim 1, wherein the expression level of the TREM-1 associated gene is increased in the presence of a natural ligand for TREM-1 but not in the presence of an agonistic anti-TREM-1 antibody.

12. The method of claim 3, wherein
the sample comprises a tissue, blood, serum, plasma, saliva, urine, or combinations thereof.

13. The method of claim 1, wherein
(a) the disease or disorder is associated with increased degranulation, reactive oxygen species formation, and/or release of pro-inflammatory cytokines by neutrophils;
(b) the disease or disorder is associated with activation of monocytes and/or increased production of inflammatory cytokines and chemokines by monocytes;
(c) the disease or disorder is associated with hypoxia; and/or
(d) the disease or disorder is associated with an increase in cell surface TREM-1 protein expression and/or an increase in level of soluble TREM-1 protein.

14. The method of claim 1, wherein the disease or disorder comprises an inflammatory bowel disease (IBD), Crohn's disease (CD), ulcerative colitis (UC), irritable bowel syndrome, rheumatoid arthritis (RA), psoriasis, psoriatic arthritis, systemic lupus erythematosus (SLE), lupus nephritis, vasculitis, sepsis, systemic inflammatory response syndrome (SIRS), type I diabetes, Grave's disease, multiple sclerosis (MS), autoimmune myocarditis, Kawasaki disease, coronary artery disease, chronic obstructive pulmonary disease, interstitial lung disease, autoimmune thyroiditis, scleroderma, systemic sclerosis, osteoarthritis, atopic dermatitis, vitiligo, graft versus host disease, Sjogrens's syndrome, autoimmune nephritis, Goodpasture's syndrome, chronic inflammatory demyelinating polyneuropathy, allergy, asthma, other autoimmune diseases that are a result of either acute or chronic inflammation, chronic kidney disease, or combinations thereof, preferably wherein the disease or disorder is inflammatory bowel disease, preferably wherein the inflammatory bowel disease comprises Crohn's disease and ulcerative colitis.

15. The method of claim 1, wherein the antagonistic anti-TREM-1 antibody comprises a heavy chain CDR1, CDR2, and CDR3 and a light chain CDR1, CDR2, and CDR3, wherein
(a) the light chain CDR1 comprises RASQSVDTFDYSFLH (SEQ ID NO: 24) or RASQSVDTFDYSFLH (SEQ ID NO: 24) except one or two substitutions,
(b) the light chain CDR2 comprises RASNLES (SEQ ID NO: 21) or RASNLES (SEQ ID NO: 21) except one or two substitutions,
(c) the light chain CDR3 comprises QQSNQDPYT (SEQ ID NO: 25) or QQSNQDPYT (SEQ ID NO: 25) except one or two substitutions,
(d) the heavy chain CDR1 comprises TYAMH (SEQ ID NO: 17) or TYAMH (SEQ ID NO: 17) except one or two substitutions,
(e) the heavy chain CDR2 comprises RIRTKSSNYATYYAASVKG (SEQ ID NO: 18 or RIRTKSSNYATYYAASVKG (SEQ ID NO: 18) except one or two substitutions, and
(f) wherein the heavy chain CDR3 comprises DMGIRRQFAY (SEQ ID NO: 19) or DMGIRRQFAY (SEQ ID NO: 19) except one or two substitutions, preferably wherein the heavy chain CDR3 comprises DQGIRRQFAY (SEQ ID NO: 72).

16. The method of claim 15, wherein the antagonistic anti-TREM-1 antibody comprises a heavy chain variable region (VH) and a light chain variable region (VL), wherein the VH comprises the amino acid sequence set forth as SEQ ID NO: 15 or 26-29 and the VL comprises the amino acid sequence set forth as SEQ ID NO: 23.

17. The method of claim 15, wherein the antagonistic anti-TREM-1 antibody comprises a heavy chain (HC) and a light chain (LC), wherein the HC comprises the amino acid sequence set forth as SEQ ID NO: 30, 31, 32, or 33, and the LC comprises the amino acid sequence set forth as SEQ ID NO: 34.

18. The method of claim 1, wherein the antagonistic anti-TREM-1 antibody comprises a heavy chain CDR1, CDR2, and CDR3 and a light chain CDR1, CDR2, and CDR3, wherein
(a) the heavy chain CDR1, CDR2, and CDR3 comprises the amino acid sequence set forth as SEQ ID NOs: 61, 62, and 63, respectively, and the light chain CDR1, CDR2, and CDR3 comprises the amino acid sequence set forth as SEQ ID NOs: 64, 65, and 66, respectively;
(b) the heavy chain CDR1, CDR2, and CDR3 comprises the amino acid sequence set forth as SEQ ID NOs: 67, 68, and 69, respectively, and the light chain CDR1, CDR2, and CDR3 comprises the amino acid sequence set forth as SEQ ID NOs: 70, 71, and 72, respectively;
(c) the heavy chain CDR1, CDR2, and CDR3 comprises the amino acid sequence set forth as SEQ ID NOs: 67, 68, and 69, respectively, and the light chain CDR1, CDR2, and CDR3 comprises the amino acid sequence set forth as SEQ ID NOs: 64, 65, and 73, respectively;
(d) the heavy chain CDR1, CDR2, and CDR3 comprises the amino acid sequence set forth as SEQ ID NOs: 74, 75, and 76, respectively, and the light chain CDR1, CDR2, and CDR3 comprises the amino acid sequence set forth as SEQ ID NOs: 70, 77, and 78, respectively;
(e) the heavy chain CDR1, CDR2, and CDR3 comprises the amino acid sequence set forth as SEQ ID NOs: 79, 80, and 81, respectively, and the light chain CDR1, CDR2, and CDR3 comprises the amino acid sequence set forth as SEQ ID NOs: 70, 71, and 72, respectively;
(f) the heavy chain CDR1, CDR2, and CDR3 comprises the amino acid sequence set forth as SEQ ID NOs: 159, 160, and 161, respectively, and the light chain CDR1, CDR2, and CDR3 comprises the amino acid sequence set forth as SEQ ID NOs: 70, 71, and 162, respectively; or
(g) the heavy chain CDR1, CDR2, and CDR3 comprises the amino acid sequence set forth as SEQ ID NOs: 159, 160, and 161, respectively, and the light chain CDR1, CDR2, and CDR3 comprises the amino acid sequence set forth as SEQ ID NOs: 70, 71, and 133, respectively.

19. The method of claim 18, wherein the antagonistic anti-TREM-1 antibody comprises a heavy chain variable region (VH) and a light chain variable region (VL), wherein the VH comprises an amino acid sequence set forth in SEQ ID NO: 53, 55, 58, 60, or 153 and wherein the VL comprises an amino acid sequence set forth in SEQ ID NO: 54, 56, 57, 59, 154, or 155.

20. The method of claim 18, wherein the antagonistic anti-TREM-1 antibody further comprises a heavy chain (HC) constant region and a light chain (LC) constant region, wherein the HC constant region comprises the amino acid sequence set forth as SEQ ID NO: 48, SEQ ID NO: 47, SEQ ID NO: 11, or SEQ ID NO: 12, and the LC constant region comprises the amino acid sequence set forth as SEQ ID NO: 35.

21. The method of claim 1, wherein the antagonistic anti-TREM-1 antibody comprises a heavy chain CDR1, CDR2, and CDR3 and a light chain CDR1, CDR2, and CDR3, wherein
(a) the heavy chain CDR1 comprises amino acids 31 to 35 (TYAMH) of SEQ ID NO: 13;
(b) the heavy chain CDR2 comprises amino acids 50 to 68 (RIRTKSSNYATYYAASVKG) of SEQ ID NO: 13;
(c) the heavy chain CDR3 comprises amino acids 101 to 110 (DMGQRRQFAY) of SEQ ID NO: 13;
(d) the light chain CDR1 comprises amino acids 24 to 38 (RASESVDTFDYSFLH) of SEQ ID NO: 14;
(e) the light chain CDR2 comprises amino acids 54 to 60 (RASNLES) of SEQ ID NO: 14; and/or
(f) the light chain CDR3 comprises amino acids 93 to 101 (QQSNEDPYT) of SEQ ID NO: 14.

22. The method of claim 21, wherein the antagonistic anti-TREM-1 antibody comprises a heavy chain variable region (VH) and a light chain variable region (VL), wherein the VH comprises amino acids 1 to 121 of SEQ ID NO: 13 and wherein the VL comprises amino acids 1 to 111 of SEQ ID NO: 14, preferably wherein the antagonistic anti-TREM-1 antibody comprises a heavy chain (HC) and a light chain (LC), wherein the HC comprises the amino acid sequence set forth as SEQ ID NO: 13 and wherein the LC comprises the amino acid sequence set forth as SEQ ID NO: 14.


SEQUENCE LISTING (295 sequences)

SEQ ID NO: 1 - protein, 234 aa, artificial sequence
  TREM-1, Isoform 1
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 2 - protein, 225 aa, artificial sequence
  TREM-1, Isoform 2
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRYSFQVPGPLVWTLSPLFPSLCAERM

SEQ ID NO: 3 - protein, 150 aa, artificial sequence
  TREM-1, Isoform 3
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFRCSTLSFSWLVDS

SEQ ID NO: 4 - DNA, 3252 bp, artificial sequence
  TREM-1, Isoform 1
agcagttggagctggtgcacaggaaggatgaggaagaccaggctctgggggctgctgtggatgctctttgtctcagaactccgagctgcaactaaattaactgaggaaaagtatgaactgaaagaggggcagaccctggatgtgaaatgtgactacacgctagagaagtttgccagcagccagaaagcttggcagataataagggacggagagatgcccaagaccctggcatgcacagagaggccttcaaagaattcccatccagtccaagtggggaggatcatactagaagactaccatgatcatggtttactgcgcgtccgaatggtcaaccttcaagtggaagattctggactgtatcagtgtgtgatctaccagcctcccaaggagcctcacatgctgttcgatcgcatccgcttggtggtgaccaagggtttttcagggacccctggctccaatgagaattctacccagaatgtgtataagattcctcctaccaccactaaggccttgtgcccactctataccagccccagaactgtgacccaagctccacccaagtcaactgccgatgtctccactcctgactctgaaatcaaccttacaaatgtgacagatatcatcagggttccggtgttcaacattgtcattctcctggctggtggattcctgagtaagagcctggtcttctctgtcctgtttgctgtcacgctgaggtcatttgtaccctaggcccacgaacccacgagaatgtcctctgacttccagccacatccatctggcagttgtgccaagggaggagggaggaggtaaaaggcagggagttaataacatgaattaaatctgtaatcaccggctatttctaaagtcagcgtctcaccttcctgcccactgccctcgttcctctaataatcttgggtgggcatttgtgcctcagaaaagaagttacagccccaaacatgcttggtccttcattccaccagccacttggggttggcatgaaatacagacagctcaatgcttttcaccgtaattctcttgtgggggctgtgacatgcagaaggcacacctgatacttctcctgctcagttttgccctggaccatacaattttggcctgacctggacagagctcccactacagaagcatcctgctcgccccatgctgggacttcctctttctagcatcagacacttgggtttcatgcttatgtgtggttctttccaacactcccagaaaagggtgttgaagattgtggaacctggagaaataagacatcgtggtgagaaagtgcatccttctcagagaaaagagttaaactgagtatcttcttctggggaaatactggcaggccgagatgggatccataggagagcaacaacagaccatgtcagacatcctgtgtgcatttatcgctggatcctgaaaatagccccgtgaaggcagaaatgtatgtgactagaacgaggccacatgaataagccactgcccactggcaggagtgaaaactgaagcgctccttacctgaaggaccccaaaaccatatagaatagaataaccaggagttccgcctgtgtctaaatgcctcttttcctgtatcacacaagggtcagggatggtggagtaaaagctctccccctgggaggcttctggaggctgtccccatgtgcttgcctagttccccactctgccctcctcctcttctctcagtctgctcctggaacacctgcctcagtttccatgctctctccagtgccctccccggtgaagcaggtaggtgttcaggccaccacagagacaatctctgtgggagattgtcttgcaatctcccacagatttcaatcaggattttgttatttcctactttgagctttaaagggaaatgggcctcatgggtggggaaaggatggtgggtccttccagcccaatttagtgatgcccagggcagatattatcctcagttcccaagagcaatcatacttttccacacataccgtgtgtctcatgttaggtaaatgtatttttacaatgagcaccacttctgtggaaaaagttccctgcacggggaggtccagcttccagactgctccatcgcataaggacttccccattcccctaaatgctgctctgtcagaacctgcccaggtaatggtaatgaccctagagagatgatttctgaaccgcaattttgagcccattagaaggtgtgtggtgggcatttatttcatcctgatgctctggtgagaatctttgcagacgcactagatccagaagctgttaatcttggtgcatttattttcctacctaaaagaaccaagcagctcagaggcagtgactgtacaggatgcagtgtttataataatgctgagcttgctggtctggaaccccacactttagcaatcccagcattgttcctgtttatgaagttgacaaagtgaccagggcaagggggtattatcattaaatacactctaggagaggcagaacacatgagggcaatgtttttcagaggtctttaggccaccgcatcagattctcctggagcataaagcaaatgctttatgagtccagggcccctgcagacctactgtatactagtatacagctccctcttagtggatctcaagcttgtttccaaaaagtcattacactccttaccaaagcccatgacacattcatacagattcatccagacataacccactgcatggtccagtgcatgcttgtgtgcttaacttattatagatcaagtgttatttaagtccaacatattaaacgtgactgaaatattatatgcactaaatcggaagacaaatggccgtgctggatactctcccatttgcacttacaggccacgccatgctccatcctccccatcctgcctgggaacctgaccaatgtggctaaaccagcaggctccctggctgccagcttctggctggttgagccaatgggaagcatgagaggagagccgagggtggggagacagtcagatcagggtttctgtcctcagggctccctccctggcaggtggtggcaggagtggctgcattcccctctgaaggctccttcaagcctctcagcaaacagctcccatctccaagtccagccacctgttccatcctctcctctgtaggcccagcagtgggaatgacccaccactattgccagcctcagcatactgaagcaccccttactggattccctaaattctatgcacatgtttattaaatgctcctcaattacccagtt

SEQ ID NO: 5 - DNA, 2128 bp, artificial sequence
  TREM-1, Isoform 2
gtgcaactttccgaagcctctaggtcattgtggtgccttgtagctgtcccgggagccctcagcagcagttggagctggtgcacaggaaggatgaggaagaccaggctctgggggctgctgtggatgctctttgtctcagaactccgagctgcaactaaattaactgaggaaaagtatgaactgaaagaggggcagaccctggatgtgaaatgtgactacacgctagagaagtttgccagcagccagaaagcttggcagataataagggacggagagatgcccaagaccctggcatgcacagagaggccttcaaagaattcccatccagtccaagtggggaggatcatactagaagactaccatgatcatggtttactgcgcgtccgaatggtcaaccttcaagtggaagattctggactgtatcagtgtgtgatctaccagcctcccaaggagcctcacatgctgttcgatcgcatccgcttggtggtgaccaagggtttttcagggacccctggctccaatgagaattctacccagaatgtgtataagattcctcctaccaccactaaggccttgtgcccactctataccagccccagaactgtgacccaagctccacccaagtcaactgccgatgtctccactcctgactctgaaatcaaccttacaaatgtgacagatatcatcaggtatagtttccaggtccctgggcccctggtttggacactgagccctttgtttcccagtctgtgtgctgagaggatgtgaaagtgagggaaaggggagggtggggcaggagaagacttgagtcacattagtctgggtagaaatgtccaggggaagaaggaagtggtgatggagaataggggaggctctcagccaggctgtccttctccccagttcaccttctttgtttccttgcaacctgagtattaaagagagggaaatggcatcttccccaagttccagtggagctcatccaaccccaggggcctgatgggcagtgggaaagcactctgagtgaggggccctggatctagtgttggcctgactaactgaatgtgactttgggtgagtcaggaaccctctctggctttagcttctttgacaattcagtaggcatggtagaaacccaaagctggaagacattgtccacctaataactctcagcaggagctggagctggggctaaatgcagtattggtttttgccttattgttttttaaataacattgttgcatgtgcccaattatagataaatagaatcagaatttttgcagatgaggtccctctcagtatttttaataagatgtttaggagatttctaatgtgtaggcaaacttaagaaccactaacttagcaatttcacacctctttacagttattaaatgttgatttatattagaatgtgtttatcattaaatactgaattatcaggatggaaatacttttccacatcaccactagtctaacatgtcttttttctcatattctcttctagtctttattcatgtttatacatattttaatatggcagtaagtctaattatacatgtttttattcttttccttgctgaaattattctttgatgctgctacctaaatttcataaatataatttcaatgagtgcatatttttctatcaattgggaaagccataacttacataaccattggcctgttcttagacattgatatagtgttgataactctgagataaacatattcttgcagatatatttttcttcttttaaataattaactcgaaatttccatgagataatttaaacactgatatcattttgtatacaaatagcatctaccaatggtcttccaaaagattggaaacaatttgcagtgtgatccattaagcataaataaagcagtgtcatggcaggcttacagacacggatttagtcttttaaatcattaaatagtgtgagttaaatcacattcctgctaaaacaaatgtgaactggtgcctgctctaatttctctatgagtgtagactccacctccatatgggtagtggcagtgcctttttccccattatgttgtttggggaacaaagtgctcattaaacttctgtggaataaatcaaacgaatgatcaaaaaaaaaaaaaaaaa

SEQ ID NO: 6 - DNA, 3140 bp, artificial sequence
  TREM-1, Isoform 3
gtgcaactttccgaagcctctaggtcattgtggtgccttgtagctgtcccgggagccctcagcagcagttggagctggtgcacaggaaggatgaggaagaccaggctctgggggctgctgtggatgctctttgtctcagaactccgagctgcaactaaattaactgaggaaaagtatgaactgaaagaggggcagaccctggatgtgaaatgtgactacacgctagagaagtttgccagcagccagaaagcttggcagataataagggacggagagatgcccaagaccctggcatgcacagagaggccttcaaagaattcccatccagtccaagtggggaggatcatactagaagactaccatgatcatggtttactgcgcgtccgaatggtcaaccttcaagtggaagattctggactgtatcagtgtgtgatctaccagcctcccaaggagcctcacatgctgttcgatcgcatccgcttggtggtgaccaaggggttccggtgttcaacattgtcattctcctggctggtggattcctgagtaagagcctggtcttctctgtcctgtttgctgtcacgctgaggtcatttgtaccctaggcccacgaacccacgagaatgtcctctgacttccagccacatccatctggcagttgtgccaagggaggagggaggaggtaaaaggcagggagttaataacatgaattaaatctgtaatcaccggctatttctaaagtcagcgtctcaccttcctgcccactgccctcgttcctctaataatcttgggtgggcatttgtgcctcagaaaagaagttacagccccaaacatgcttggtccttcattccaccagccacttggggttggcatgaaatacagacagctcaatgcttttcaccgtaattctcttgtgggggctgtgacatgcagaaggcacacctgatacttctcctgctcagttttgccctggaccatacaattttggcctgacctggacagagctcccactacagaagcatcctgctcgccccatgctgggacttcctctttctagcatcagacacttgggtttcatgcttatgtgtggttctttccaacactcccagaaaagggtgttgaagattgtggaacctggagaaataagacatcgtggtgagaaagtgcatccttctcagagaaaagagttaaactgagtatcttcttctggggaaatactggcaggccgagatgggatccataggagagcaacaacagaccatgtcagacatcctgtgtgcatttatcgctggatcctgaaaatagccccgtgaaggcagaaatgtatgtgactagaacgaggccacatgaataagccactgcccactggcaggagtgaaaactgaagcgctccttacctgaaggaccccaaaaccatatagaatagaataaccaggagttccgcctgtgtctaaatgcctcttttcctgtatcacacaagggtcagggatggtggagtaaaagctctccccctgggaggcttctggaggctgtccccatgtgcttgcctagttccccactctgccctcctcctcttctctcagtctgctcctggaacacctgcctcagtttccatgctctctccagtgccctccccggtgaagcaggtaggtgttcaggccaccacagagacaatctctgtgggagattgtcttgcaatctcccacagatttcaatcaggattttgttatttcctactttgagctttaaagggaaatgggcctcatgggtggggaaaggatggtgggtccttccagcccaatttagtgatgcccagggcagatattatcctcagttcccaagagcaatcatacttttccacacataccgtgtgtctcatgttaggtaaatgtatttttacaatgagcaccacttctgtggaaaaagttccctgcacggggaggtccagcttccagactgctccatcgcataaggacttccccattcccctaaatgctgctctgtcagaacctgcccaggtaatggtaatgaccctagagagatgatttctgaaccgcaattttgagcccattagaaggtgtgtggtgggcatttatttcatcctgatgctctggtgagaatctttgcagacgcactagatccagaagctgttaatcttggtgcatttattttcctacctaaaagaaccaagcagctcagaggcagtgactgtacaggatgcagtgtttataataatgctgagcttgctggtctggaaccccacactttagcaatcccagcattgttcctgtttatgaagttgacaaagtgaccagggcaagggggtattatcattaaatacactctaggagaggcagaacacatgagggcaatgtttttcagaggtctttaggccaccgcatcagattctcctggagcataaagcaaatgctttatgagtccagggcccctgcagacctactgtatactagtatacagctccctcttagtggatctcaagcttgtttccaaaaagtcattacactccttaccaaagcccatgacacattcatacagattcatccagacataacccactgcatggtccagtgcatgcttgtgtgcttaacttattatagatcaagtgttatttaagtccaacatattaaacgtgactgaaatattatatgcactaaatcggaagacaaatggccgtgctggatactctcccatttgcacttacaggccacgccatgctccatcctccccatcctgcctgggaacctgaccaatgtggctaaaccagcaggctccctggctgccagcttctggctggttgagccaatgggaagcatgagaggagagccgagggtggggagacagtcagatcagggtttctgtcctcagggctccctccctggcaggtggtggcaggagtggctgcattcccctctgaaggctccttcaagcctctcagcaaacagctcccatctccaagtccagccacctgttccatcctctcctctgtaggcccagcagtgggaatgacccaccactattgccagcctcagcatactgaagcaccccttactggattccctaaattctatgcacatgtttattaaatgctcctcaattacccagttaaaaaaaaaaaaaaaaaa

SEQ ID NO: 7 - protein, 233 aa, artificial sequence
  TREM-1 (Predicted)
MRKTRLWGLLWMLFVSELRATTELTEEKYEYKEGQTLEVKCDYALEKYANSRKAWQKMEGKMPKILAKTERPSENSHPVQVGRITLEDYPDHGLLQVQMTNLQVEDSGLYQCVIYQHPKESHVLFNPICLVVTKGSSGTPGSSENSTQNVYRTPSTTAKALGPRYTSPRTVTQAPPESTVVVSTPGSEINLTNVTDIIRVPVFNIVIIVAGGFLSKSLVFSVLFAVTLRSFGP

SEQ ID NO: 8 - protein, 196 aa, artificial sequence
  peptidoglycan recognition protein 1
MSRRSMLLAWALPSLLRLGAAQETEDPACCSPIVPRNEWKALASECAQHLSLPLRYVVVSHTAGSSCNTPASCQQQARNVQHYHMKTLGWCDVGYNFLIGEDGLVYEGRGWNFTGAHSGHLWNPMSIGISFMGNYMDRVPTPQAIRAAQGLLACGVAQGALRSNYVLKGHRDVQRTLSPGNQLYHLIQNWPHYRSP

SEQ ID NO: 9 - protein, 330 aa, artificial sequence [sequence omitted]

SEQ ID NO: 10 - protein, 327 aa, artificial sequence
  WT IgG4 Heavy Chain Constant Region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 330 aa, artificial sequence
  IgG1-Aba Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 330 aa, artificial sequence
  IgG4-Aba Heavy Chain Constant Region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 448 aa, artificial sequence
  HC of mAb 0170
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 218 aa, artificial sequence
  LC of mAb 0170
DIVLTQSPDSLAVSLGERATINCRASESVDTFDYSFLHWYQQKPGQPPKLLIYRASNLESGVPDRFSGSGSGTDFTLTISSLQAEDVAVYYCQQSNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 121 aa, artificial sequence
  VH of mAb 0170, 0318
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSS

SEQ ID NO: 16 - protein, 111 aa, artificial sequence
  VL of mAb 0170
DIVLTQSPDSLAVSLGERATINCRASESVDTFDYSFLHWYQQKPGQPPKLLIYRASNLESGVPDRFSGSGSGTDFTLTISSLQAEDVAVYYCQQSNEDPYTFGQGTKLEIK

SEQ ID NO: 17 - protein, 5 aa, artificial sequence
  CDR1 VH of mAb 0170, 0318
TYAMH

SEQ ID NO: 18 - protein, 19 aa, artificial sequence
  CDR2 VH of mAb 0170, 0318
RIRTKSSNYATYYAASVKG

SEQ ID NO: 19 - protein, 10 aa, artificial sequence
  CDR3 VH of mAb 0170, 0318
DMGIRRQFAY

SEQ ID NO: 20 - protein, 15 aa, artificial sequence
  CDR1 VL of mAb 0170
RASESVDTFDYSFLH

SEQ ID NO: 21 - protein, 7 aa, artificial sequence
  CDR2 VL of mAb 0170, 0318
RASNLES

SEQ ID NO: 22 - protein, 9 aa, artificial sequence
  CDR3 VL of mAb 0170
QQSNEDPYT

SEQ ID NO: 23 - protein, 111 aa, artificial sequence
  VL of mAb 0318
DIVLTQSPDSLAVSLGERATINCRASQSVDTFDYSFLHWYQQKPGQPPKLLIYRASNLESGVPDRFSGSGSGTDFTLTISSLQAEDVAVYYCQQSNQDPYTFGQGTKLEIK

SEQ ID NO: 24 - protein, 15 aa, artificial sequence
  CDR1 VL of mAb 0318
RASQSVDTFDYSFLH

SEQ ID NO: 25 - protein, 9 aa, artificial sequence
  CDR3 VL of mAb 0318
QQSNQDPYT

SEQ ID NO: 26 - protein, 121 aa, artificial sequence
  VH of 318 Ab - Mutant #1
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDQGIRRQFAYWGQGTLVTVSS

SEQ ID NO: 27 - protein, 121 aa, artificial sequence
  VH of 318 Ab - Mutant #2
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDLGIRRQFAYWGQGTLVTVSS

SEQ ID NO: 28 - protein, 121 aa, artificial sequence
  VH of 318 Ab - Mutant #3
EVQLVESGGGLVQPGGSLKLSCAASGFTFSTYAQHWVRQASGKGLEWVGRIRTKSSNYATYYAASVKGRFTISRDDSKNTAYLQMNSLKTEDTAVYYCTRDMGIRRQFAYWGQGTLVTVSS

SEQ ID NO: 29 - protein, 121 aa, artificial sequence
  VH of 318 Ab - Mutant #4
EVQLVESGGGLVQPGGSLKLSCAASGFTFSTYALHWVRQASGKGLEWVGRIRTKSSNYATYYAASVKGRFTISRDDSKNTAYLQMNSLKTEDTAVYYCTRDMGIRRQFAYWGQGTLVTVSS

SEQ ID NO: 30 - protein, 451 aa, artificial sequence
  0318-IgG1.3f HC
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 450 aa, artificial sequence
  0318-IgG1.1f HC
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 451 aa, artificial sequence
  0318-IgG1-Aba HC
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 451 aa, artificial sequence
  0318-IgG4-Aba HC
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 218 aa, artificial sequence
  0318-IgG1.3f LC; 0318-IgG1.1f LC; 0318-IgG1-Aba LC; 0318-IgG4-Aba LC
DIVLTQSPDSLAVSLGERATINCRASQSVDTFDYSFLHWYQQKPGQPPKLLIYRASNLESGVPDRFSGSGSGTDFTLTISSLQAEDVAVYYCQQSNQDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 107 aa, artificial sequence [sequence omitted]

SEQ ID NO: 36 - protein, 1407 aa, artificial sequence
  0318-IgG1.3f HC
ATGAGGGCTTGGATCTTCTTTCTGCTCTGCCTGGCCGGGCGCGCCTTGGCCGAAGTGCAGCTCGTGGAGTCCGGCGGAGGACTGGTCCAACCTGGCGGCTCCCTGAAGCTGTCCTGCGCTGCCTCCGGCTTCACCTTCTCCACCTACGCCATGCACTGGGTCAGGCAGGCCTCCGGAAAGGGCCTGGAATGGGTCGGCCGGATTAGGACCAAGTCCAGCAACTACGCTACCTACTACGCCGCCAGCGTCAAGGGCCGGTTCACAATCTCCCGGGACGACTCCAAGAACACCGCCTATCTCCAGATGAACAGCCTGAAGACAGAGGACACCGCCGTGTACTATTGCACCCGGGATATGGGCATTCGGAGGCAGTTCGCCTATTGGGGCCAGGGCACCCTGGTGACAGTCAGCTCCGCCAGCACAAAAGGACCTAGCGTGTTCCCCCTGGCCCCTAGCAGCAAGTCCACAAGCGGCGGCACCGCTGCCCTGGGCTGTCTGGTGAAAGACTACTTTCCCGAGCCCGTGACAGTGAGCTGGAACTCTGGCGCCCTGACATCCGGAGTGCACACCTTCCCTGCCGTGCTCCAGTCCAGCGGCCTGTACAGCCTGAGCAGCGTCGTGACCGTCCCTAGCAGCAGCCTGGGAACCCAGACCTACATCTGCAACGTGAACCACAAGCCCTCCAACACCAAGGTGGACAAGAGGGTGGAGCCCAAGTCCTGTGACAAGACCCATACCTGCCCCCCCTGTCCTGCTCCTGAAGCTGAGGGCGCCCCTTCCGTCTTCCTGTTCCCTCCTAAGCCCAAGGACACCCTGATGATCTCCAGGACCCCCGAGGTGACCTGTGTGGTGGTGGATGTGTCCCACGAGGACCCCGAGGTGAAGTTCAATTGGTACGTCGACGGCGTGGAGGTGCACAACGCCAAGACCAAACCCCGGGAGGAGCAGTATAACAGCACCTACCGGGTGGTGTCCGTGCTCACCGTGCTGCACCAGGACTGGCTGAACGGCAAGGAGTACAAGTGTAAGGTCAGCAATAAGGCCCTGCCTGCCCCCATCGAGAAGACCATTAGCAAGGCTAAGGGCCAGCCCAGGGAACCCCAGGTGTATACCCTCCCCCCTAGCCGGGAGGAGATGACCAAGAACCAGGTCAGCCTGACCTGCCTGGTGAAAGGATTCTACCCTAGCGACATCGCTGTGGAGTGGGAGAGCAATGGACAGCCCGAGAACAACTACAAAACCACCCCTCCCGTCCTGGACTCCGATGGCAGCTTCTTTCTGTACTCCAAGCTCACCGTCGACAAGAGCCGGTGGCAGCAGGGCAATGTGTTTAGCTGCTCCGTGATGCACGAGGCTCTGCACAACCACTATACCCAAAAGTCCCTGTCCCTCAGCCCCGGCAAGTGA

SEQ ID NO: 37 - protein, 1404 aa, artificial sequence
  0318-IgG1.1f HC
ATGAGGGCTTGGATCTTCTTTCTGCTCTGCCTGGCCGGGCGCGCCTTGGCCGAAGTGCAGCTGGTGGAAAGCGGCGGAGGACTGGTGCAGCCAGGCGGCAGCCTGAAGCTGTCTTGCGCCGCCAGCGGCTTCACCTTCAGCACCTACGCCATGCACTGGGTCCGCCAGGCCAGCGGCAAGGGCCTGGAATGGGTCGGACGGATCCGGACCAAGAGCAGCAACTACGCCACCTACTACGCCGCCTCCGTGAAGGGCCGGTTCACCATCAGCCGGGACGACAGCAAGAACACCGCCTACCTGCAGATGAACAGCCTGAAAACCGAGGACACCGCCGTGTACTACTGCACCCGGGACATGGGCATCCGGCGGCAGTTTGCCTACTGGGGCCAGGGCACCCTGGTCACAGTGTCCAGCGCGTCGACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAGAGTTGAGCCCAAATCTTGTGACAAAACTCACACATGCCCACCGTGCCCAGCACCTGAAGCCGAAGGGGCCCCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAAGCAGCATCGAGAAAACCATCTCCAAAGCCAAAGGGCAGCCCCGAGAACCACAGGTGTACACCCTGCCCCCATCCCGGGAGGAGATGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCCCCGGGTTGA

SEQ ID NO: 38 - protein, 1407 aa, artificial sequence
  0318-IgG1-Aba HC
ATGAGGGCTTGGATCTTCTTTCTGCTCTGCCTGGCCGGGCGCGCCTTGGCCGAGGTCCAGCTGGTGGAGAGCGGAGGAGGACTCGTGCAGCCTGGAGGATCCCTGAAGCTGTCCTGCGCTGCCTCCGGCTTTACCTTCTCCACCTACGCCATGCATTGGGTGAGGCAGGCCTCCGGCAAGGGCCTGGAATGGGTGGGCAGGATCAGGACAAAGAGCTCCAACTACGCCACCTACTACGCCGCCAGCGTGAAAGGACGGTTCACCATCTCCCGGGACGATTCCAAAAACACCGCCTACCTCCAGATGAATAGCCTGAAGACCGAGGACACCGCCGTCTACTACTGCACCAGGGATATGGGCATCCGGCGGCAGTTTGCCTACTGGGGCCAGGGCACCCTGGTGACAGTGTCCAGCGCTAGCACCAAAGGCCCCTCCGTGTTCCCCCTGGCTCCCTCCAGCAAGTCCACATCCGGCGGAACCGCCGCTCTGGGATGTCTGGTGAAGGACTACTTCCCCGAGCCCGTGACAGTGAGCTGGAACTCTGGCGCTCTGACCTCCGGCGTGCACACCTTTCCTGCTGTGCTGCAGTCCTCCGGCCTCTACAGCCTGTCCTCCGTCGTGACAGTGCCCTCCTCCAGCCTGGGCACCCAGACCTACATCTGCAACGTGAACCACAAGCCTTCCAACACCAAGGTGGATAAGCGGGTCGAACCCAAGAGCTGCGACAAGACCCACACCAGCCCCCCTTCCCCTGCTCCCGAGCTCCTGGGAGGCAGCTCCGTGTTTCTGTTTCCCCCCAAGCCTAAGGACACCCTGATGATCAGCAGGACCCCCGAAGTGACATGCGTGGTGGTCGACGTGTCCCACGAGGACCCCGAGGTCAAGTTCAACTGGTACGTGGATGGAGTGGAGGTCCATAATGCCAAGACCAAGCCCCGGGAGGAGCAGTACAATTCCACCTACCGGGTGGTGTCCGTGCTGACAGTCCTGCATCAGGACTGGCTCAACGGCAAGGAGTACAAATGCAAGGTGTCCAACAAGGCTCTGCCCGCCCCCATCGAGAAGACAATCAGCAAGGCTAAGGGCCAGCCTAGGGAGCCCCAGGTGTACACCCTGCCCCCTTCCAGGGACGAGCTCACCAAGAACCAGGTGTCCCTGACATGCCTCGTGAAGGGCTTTTACCCTAGCGACATCGCTGTGGAGTGGGAGTCCAACGGACAGCCCGAGAACAACTACAAGACAACACCCCCTGTGCTGGACTCCGACGGCTCCTTCTTCCTGTACAGCAAGCTCACCGTGGACAAATCCCGGTGGCAGCAGGGAAACGTGTTCAGCTGCTCCGTGATGCATGAGGCCCTGCACAACCATTACACCCAGAAATCCCTGTCCCTGTCCCCCGGCAAGTGA

SEQ ID NO: 39 - protein, 1407 aa, artificial sequence
  0318-IgG4-Aba HC
ATGAGGGCTTGGATCTTCTTTCTGCTCTGCCTGGCCGGGCGCGCCTTGGCCGAGGTCCAGCTGGTCGAGAGCGGCGGAGGACTGGTCCAGCCTGGAGGATCCCTGAAGCTCTCCTGTGCCGCTAGCGGATTTACCTTCAGCACCTACGCTATGCATTGGGTGAGGCAGGCTTCCGGCAAGGGACTGGAGTGGGTGGGACGGATTCGGACCAAGAGCTCCAATTACGCCACCTATTACGCCGCTTCCGTGAAGGGCCGGTTCACCATCTCCAGGGACGACTCCAAGAACACCGCTTACCTGCAGATGAACTCCCTCAAGACCGAGGACACCGCCGTGTATTACTGCACCCGGGACATGGGCATCCGGAGGCAGTTCGCTTATTGGGGACAGGGCACCCTGGTGACCGTGAGCTCCGCCTCCACCAAGGGCCCTTCCGTGTTCCCTCTGGCCCCCTGTAGCAGGTCCACCAGCGAGTCCACAGCTGCTCTGGGCTGTCTGGTGAAGGATTATTTCCCCGAGCCCGTGACAGTGTCCTGGAATAGCGGCGCCCTGACCTCCGGCGTGCATACCTTCCCTGCCGTCCTGCAAAGCAGCGGCCTGTACAGCCTGTCCTCCGTGGTGACAGTCCCTTCCTCCAGCCTGGGAACCAAGACCTACACCTGCAATGTGGACCACAAGCCTAGCAACACCAAGGTGGACAAGAGGGTCGAGCCCAAGTCCTGCGATAAGACACACACCTCCCCTCCTTCCCCTGCTCCTGAGCTGCTCGGCGGAAGCAGCGTGTTCCTGTTCCCTCCTAAGCCTAAGGACACCCTGATGATCAGCCGGACCCCTGAGGTGACCTGTGTCGTGGTGGACGTGTCCCACGAAGACCCCGAGGTGAAATTCAACTGGTACGTGGACGGCGTCGAGGTGCACAACGCTAAGACAAAACCCAGGGAGGAGCAGTACAATTCCACCTACCGGGTCGTGTCCGTGCTGACCGTGCTGCACCAGGACTGGCTGAACGGCAAAGAGTACAAGTGTAAGGTCAGCAATAAGGCCCTGCCTGCCCCCATTGAGAAGACCATTTCCAAGGCCAAGGGCCAGCCCAGGGAACCTCAGGTGTACACCCTGCCTCCCTCCCGGGACGAGCTGACCAAGAACCAGGTCAGCCTGACCTGCCTCGTGAAGGGCTTCTACCCTAGCGACATCGCCGTGGAGTGGGAGTCCAACGGCCAGCCTGAGAACAATTACAAGACCACCCCCCCTGTCCTGGACTCCGACGGATCCTTCTTCCTGTACTCCAAGCTGACAGTGGATAAGTCCCGGTGGCAGCAGGGAAATGTGTTCTCCTGCTCCGTCATGCACGAAGCCCTGCATAACCACTACACACAGAAGAGCCTGTCCCTGAGCCCTGGCAAGTGA

SEQ ID NO: 40 - protein, 708 aa, artificial sequence
  0318-IgG1.3f LC; 0318-IgG1.1f LC; 0318-IgG1-Aba LC; 0318-IgG4-Aba LC
ATGAGGGCTTGGATCTTCTTTCTGCTCTGCCTGGCCGGGCGCGCCTTGGCCGATATCGTGCTGACCCAGAGCCCCGATAGCCTGGCTGTGAGCCTGGGCGAGAGAGCCACCATCAACTGCAGGGCCTCCCAGAGCGTCGACACCTTCGACTACAGCTTCCTCCACTGGTACCAGCAGAAGCCTGGCCAGCCCCCCAAGCTGCTGATCTACAGGGCCAGCAACCTGGAGAGCGGAGTGCCCGATAGGTTCAGCGGCAGCGGCTCCGGAACCGACTTTACCCTCACCATCAGCTCCCTGCAGGCCGAGGATGTGGCCGTCTACTACTGCCAGCAGAGCAACCAGGACCCCTACACCTTTGGCCAGGGCACCAAGCTGGAGATCAAGAGGACCGTGGCCGCCCCCTCCGTGTTCATCTTCCCTCCCAGCGACGAGCAGCTGAAGAGCGGAACCGCCAGCGTGGTGTGCCTGCTGAACAACTTCTACCCCAGGGAGGCCAAGGTGCAGTGGAAGGTCGACAACGCCCTGCAGAGCGGCAATAGCCAGGAGAGCGTGACCGAACAGGACAGCAAGGACTCCACCTACTCCCTGAGCAGCACCCTGACCCTGAGCAAGGCCGACTACGAGAAGCACAAGGTGTACGCCTGTGAGGTGACCCACCAGGGACTGAGCAGCCCCGTGACAAAGAGCTTTAACAGGGGCGAGTGCTGA

SEQ ID NO: 41 - protein, 10 aa, artificial sequence
  0318 AB - VH CDR3 (mutant #1)
DQGIRRQFAY

SEQ ID NO: 42 - protein, 10 aa, artificial sequence
  0318 AB - VH CDR3 (mutant #2)
DLGIRRQFAY

SEQ ID NO: 43 - protein, 5 aa, artificial sequence
  0318 Ab - VH CDR1 (mutant #3)
TYAQH

SEQ ID NO: 44 - protein, 5 aa, artificial sequence
  0318 Ab - VH CDR1 (mutant #4)
TYALH

SEQ ID NO: 45 - protein, 451 aa, artificial sequence
  0318 Ab - HC (IgG4)
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNDIVLTQSPDSLAVSLG

SEQ ID NO: 46 - protein, 330 aa, artificial sequence
  WT IgG1 Constant Region (Allotypic Variant)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 330 aa, artificial sequence
  IgG1.1f HC Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 330 aa, artificial sequence
  IgG1.3f HC Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 5 aa, artificial sequence
  Linker
PVGVV

SEQ ID NO: 50 - protein, 11 aa, artificial sequence
  Amino Acid Residues 27-37 of Human TREM-1, Isoform 1
EKYELKEGQTL

SEQ ID NO: 51 - protein, 13 aa, artificial sequence
  Amino Acid Residues 88-100 of Human TREM-1, Isoform 1
EDYHDHGLLRVRM

SEQ ID NO: 52 - protein, 9 aa, artificial sequence
  Amino Acid Residues 120-128 of Human TREM-1, Isoform 1
KEPHMLFDR

SEQ ID NO: 53 - protein, 124 aa, artificial sequence
  P1-047248 VH
QVQLQESGPGLVKPSETLSLTCTVSGGSISSSYWSWVRQPPGKGLEWIGYTHYSGISNYNPSLKSRVTISVDTSKNQFSLKLSSVTAADTAVYYCAREGYDILTGYEYYGMDVWGQGTTVTVSS

SEQ ID NO: 54 - protein, 107 aa, artificial sequence
  P1-047248 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGGGTKVEIK

SEQ ID NO: 55 - protein, 122 aa, artificial sequence
  P1-047246, P1-047247 VH
QVQLQESGPGLVKPSETLSLTCTVSGGSITNYYWTWIRQPPGKGLEWIGYIYDSGYTNYNPSLKSRVTLSIDTSKNQFSLKLSSVTAADTAVYYCARGVLWFGELLPLLDYWGQGTLVTVSS

SEQ ID NO: 56 - protein, 107 aa, artificial sequence
  P1-047246, P1-047239 VL
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 57 - protein, 108 aa, artificial sequence
  P1-047247 VL
EIVLTQSPGTLSLSPGERATLSCRASQSVSSSYLAWYQQKPGQAPRLLIYGASSRATGIPERFSGSGSGTDFTLTISRLEPEDFAVYYCQQYGSSPLTFGGGTKVEIK

SEQ ID NO: 58 - protein, 123 aa, artificial sequence
  P1-047334 VH
QVQLVQSGAEVKRPGSSVKVSCKASGGTFSSSAISWVRQAPGQGLEWMGGIIPIFGTTNGAQKFQGRVTITADESTSTAYMELSSLRSEDTAVYYCAAMVRGNYFYFYGMDVWGQGTTVTVSS

SEQ ID NO: 59 - protein, 107 aa, artificial sequence
  P1-047334 VL
AIQLTQSPSSLSASVGDRVTITCRASQGISSALAWYQQKPGKAPKLLIYAASSLQSGVPSRFSGSGSGTDFTLTISSLQPEDFATYYCQQYNSYPLTFGGGTKVEIK

SEQ ID NO: 60 - protein, 125 aa, artificial sequence
  P1-047239 VH
QVQLVESGGGVVQPGRSLRLSCAATEFTFSNYGMHWVRQAPGKGLEWVAVIWYDGSNKYYADSVKGRFTISRDNSKNTLYLQLNSLSAEDSAVYYCARDGRHYYGSTSYFGMDVWGQGTTVTVSS

SEQ ID NO: 61 - protein, 5 aa, artificial sequence
  P1-047248 HCDR1
SSYWS

SEQ ID NO: 62 - protein, 16 aa, artificial sequence
  P1-047248 HCDR2
YTHYSGISNYNPSLKS

SEQ ID NO: 63 - protein, 16 aa, artificial sequence
  P1-047248 HCDR3
EGYDILTGYEYYGMDV

SEQ ID NO: 64 - protein, 12 aa, artificial sequence
  P1-047248, P1-047247 LCDR1
RASQSVSSSYLA

SEQ ID NO: 65 - protein, 7 aa, artificial sequence
  P1-047248, P1-047247, P1-047317 LCDR2
GASSRAT

SEQ ID NO: 66 - protein, 8 aa, artificial sequence
  P1-047248 LCDR3
QQYGSSPT

SEQ ID NO: 67 - protein, 5 aa, artificial sequence
  P1-047246, P1-047247 HCDR1
NYYWT

SEQ ID NO: 68 - protein, 16 aa, artificial sequence
  P1-047246, P1-047247 HCDR2
YIYDSGYTNYNPSLKS

SEQ ID NO: 69 - protein, 14 aa, artificial sequence
  P1-047246, P1-047247 HCDR3
GVLWFGELLPLLDY

SEQ ID NO: 70 - protein, 11 aa, artificial sequence
  P1-047246, P1-047334, P1-047239, P1-047323, P1-047328 LCDR1
RASQGISSALA

SEQ ID NO: 71 - protein, 7 aa, artificial sequence
  P1-047246, P1-047239, P1-047323, P1-047328 LCDR2
DASSLES

SEQ ID NO: 72 - protein, 9 aa, artificial sequence
  P1-047246, P1-047239 LCDR3
QQFNSYPYT

SEQ ID NO: 73 - protein, 9 aa, artificial sequence
  P1-047247, P1-047317 LCDR3
QQYGSSPLT

SEQ ID NO: 74 - protein, 5 aa, artificial sequence
  P1-047334, P1-047292, P1-047289, P1-047306 HCDR1
SSAIS

SEQ ID NO: 75 - protein, 17 aa, artificial sequence
  P1-047334 HCDR2
GIIPIFGTTNGAQKFQG

SEQ ID NO: 76 - protein, 14 aa, artificial sequence
  P1-047334 HCDR3
MVRGNYFYFYGMDV

SEQ ID NO: 77 - protein, 7 aa, artificial sequence
  LCDR2
AASSLQS

SEQ ID NO: 78 - protein, 9 aa, artificial sequence
  LCDR3
QQYNSYPLT

SEQ ID NO: 79 - protein, 5 aa, artificial sequence
  P1-047239 HCDR1
NYGMH

SEQ ID NO: 80 - protein, 17 aa, artificial sequence
  P1-047239 HCDR2
VIWYDGSNKYYADSVKG

SEQ ID NO: 81 - protein, 16 aa, artificial sequence
  P1-047239 HCDR3
DGRHYYGSTSYFGMDV

SEQ ID NO: 82 - protein, 126 aa, artificial sequence
  P1-047305 VH
QVQLVQSGAEVKKPGSSVKVSCKTSGGTFSSSAVSWVRQAPGQGLEWMGGITPIFGTADYAQKFQGRVTITADASTSTGYMELSSLRSEDTAVYYCAFTPRYRGSSHHYYYALGVWGQGTTVTVSS

SEQ ID NO: 83 - protein, 107 aa, artificial sequence
  P1-047305, P1-047314, P1-047290, P1-047308, P1-047292, P1-047322, P1-047301, P1-047265 VL
DIQMTQSPTSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 84 - protein, 117 aa, artificial sequence
  P1-047309 VH
QVQLVQSGAEVKKPGSSVKVSCNPSGGTFSTYAISWVRQAPGQGLEWMGGINPIFGTANYAQKFQGRVTITADESTSPGYLELSSLRSEDTAVYYCARGGAVGFAYWGQGTLVTVSS

SEQ ID NO: 85 - protein, 107 aa, artificial sequence
  P1-047309 VL
DIQMTQSPSSVSASVGDRVTITCRASQGISSWLAWYQHKPGKAPKLLIYAASSLQSGVPSRFSGSGSGTDFTLTISSLQPEDFATYYCQQANSFPFTFGGGTKVEIK

SEQ ID NO: 86 - protein, 125 aa, artificial sequence
  P1-047313 VH
QVQLVQSGAEVKRPGSSVKVSCKASGGTFSIYVISWVRQAPGQGLEWMGGIIPLFGTPNYAQRFQDRVTITADESTRTAYMELSSLRSEDTAVYYCARGHGPGSSHYSYYGLDVWGQGTTVTVSS

SEQ ID NO: 87 - protein, 107 aa, artificial sequence
  P1-047313 VL
DIQMTQSPTSLSASVGDRVTITCRASQGISSWLAWYQHKPGKAPKLLIYAASSLQSGVPSRFSGSGSGTDFTLTISSLQPEDFATYYCQQYNSYPWTFGQGTKVEIK

SEQ ID NO: 88 - protein, 125 aa, artificial sequence
  P1-047307, P1-047320, P1-047314, P1-047312, P1-047317 VH
QVQLVQSGAEVKRPGSSVKVSCKASGGTFSIYVISWVRQAPGQGLEWMGGIIPLFGTPNYAQQFQDRVTITADESTRTAYMELNSLKSEDTAVYYCARGHGPGSSHYSYYGLDVWGQGTTVTVSS

SEQ ID NO: 89 - protein, 107 aa, artificial sequence
  P1-047307, P1-047302, P1-047306 VL
DIQMTQSPTSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 90 - protein, 107 aa, artificial sequence
  P1-047312, P1-047318 VL
DIQMTQSPTSLSASVGDRVTITCRASQGISSWLAWYQHKPGKAPKLLIYAASSLQSGVPSRFSGSGSGTDFTLTISSLQPEDFATYYCQQYNSYPYTFGQGTKLEIK

SEQ ID NO: 91 - protein, 125 aa, artificial sequence
  P1-047318 VH
QVQLVQSGAEVKRPGSSVKVSCKASGGTFSIYVISWVRQAPGQGLEWMGGIIPLFGTPNYAQQFQDRVTITADESTRTAYMELSSLRSEDTAVYYCARGHGPGSSHYSYYGLDVWGQGTTVTVSS

SEQ ID NO: 92 - protein, 107 aa, artificial sequence
  P1-047320, P1-047319 VL
DIQMTQSPTSLSASVGDRVTITCRASQGISSWLAWYQHKPGKAPKSLIYAASSLQSGVPSRFSGSGSGTDFTLTISSLQPEDFATYYCQQYNSYPYTFGQGTKLEIK

SEQ ID NO: 93 - protein, 125 aa, artificial sequence
  P1-047311 VH
QVQLVQSGAEVKKPGSSVKVSCKASGGTFSIYVISWVRQAPGQGLEWMGGIIPLFGTANYAQQFQDRVTITADESTRTAYMELNSLKSEDTAVYYCARGHGPGSSHYSYYGLDVWGQGTTVTVSS

SEQ ID NO: 94 - protein, 107 aa, artificial sequence
  P1-047311 VL
DIQMTQSPTSLSASVGDRVTITCRASQGISSWLAWYQQKPGKAPKSLIYAASSLQSGVPSRFSGSGSGTDFTLTISSLQPEDFATYYCQQYNSYPLTFGGGTKVEIK

SEQ ID NO: 95 - protein, 127 aa, artificial sequence
  P1-047294 VH
QVQLVQSGAEVKKPGSSVKVSCKASGGTFNRHAISWVRQAPGQGLEWMGGIIPLFGTANYAQEFQGRVTIAADEPTSTTYMELRSLRSEDTAVYYCASSYFYGSGSSNYYYYGLDVWGQGTTVTVSS

SEQ ID NO: 96 - protein, 107 aa, artificial sequence
  P1-047294 VL
DIQMTQSPTSLSASVGDRVTITCRASQGISSWLAWYQQKPEKAPKSLIYAASSLQSGVPSRFSGGGSGTDFTLTISSLQPEDFATYYCQQYNSYPLTFGGGTKVEIK

SEQ ID NO: 97 - protein, 127 aa, artificial sequence
  P1-047290, P1-047291, P1-047296, P1-047297, P1-047300, P1-047302 VH
QVQLVQSGAEVKKPGSSVKVSCKASGGTFNRHAISWVRQAPGQGLEWMGGIIPLFGTANYAQKFQGRVTIAADEPTSTTYMELRSLRSEDTAVYYCASSYFYGSGSSNYYYYGLDVWGQGTTVTVSS

SEQ ID NO: 98 - protein, 107 aa, artificial sequence
  P1-047291 VL
DIQMTQSPTSLSASVGDRVTITCRASQGISSWLAWYQQKPEKAPKSLIYAASSLQSGVPSRFSGSGSGTDFTLTISSLQPDDFATYYCQQYNSYPYTFGQGTKLEIK

SEQ ID NO: 99 - protein, 107 aa, artificial sequence
  P1-047296 VL
DIQMTQSPTSLSASVGDRVTITCRASQGISSWLAWYQQKPEKAPKSLIYAASSLQSGVPSRFSGSGSGTDFTLTISSLQPEDFATYYCQQYNSYPITFGQGTRLEIK

SEQ ID NO: 100 - protein, 107 aa, artificial sequence
  P1-047297 VL
DIQMTQSPTSLSASVGDRVTITCRASQGISSWLAWYQHKPGKAPKSLIYAASSLQSGVPSRFSGSGSGTDFTLTISSLQPEDFATYYCQQYNSYPLTFGGGTKVEIK

SEQ ID NO: 101 - protein, 107 aa, artificial sequence
  P1-047300 VL
DIQMTQSPTSLSASVGDRVTITCRASQGISSWLAWYQQKPEKAPKSLIHAASSLQSGVPSRFSGSGSGTDFTLTISSLQPEDFATYYCQQYNSYPLTFGGGTKVEIK

SEQ ID NO: 102 - protein, 125 aa, artificial sequence
  P1-047308, P1-047319 VH
QVQLVQSGAEVKRPGSSVKVSCKASGGTFSIYVISWVRQAPGQGLEWMGGIIPLFGTANYAQKFQGRVTITADESTNTAYMELSSLRSEDTAVYYCARGHGPGSSHYSYYGLDVWGQGTTVTVSS

SEQ ID NO: 103 - protein, 126 aa, artificial sequence
  P1-047292 VH
QVQLVQSGAEVKKPGSSVKVSCKASGGTFSSSAISWVRQAPGQGLEWMGGIIPIFSTGNYAQKFQGRVTITADESTNTAYMDLSSLRSEDTAVYYCARSTRVRGVSHYYYYGLDVWGQGTTVTVSS

SEQ ID NO: 104 - protein, 127 aa, artificial sequence
  P1-047322 VH
QVQLVQSGAEVKKPGSSVKVSCKSSGGTFSSYAFTWVRQAPGQGLEWMGGIIPIFRTANYAQKFQGRVTITADESTSTAYMELSSLRSEDTAVYYCASSHFSGSGSSHYYYYGMHVWGQGTTVTVSS

SEQ ID NO: 105 - protein, 125 aa, artificial sequence
  P1-047310 VH
QVQLVQSGAEVKKPGSSVKVSCKASGGTFSRYAISWVRQAPGQGLEWMGGIIPIFGTSNYAQKFQGRVTIKADESTSTAYMELSSLRSEDTAVYYCARGGNSWTTSLYYYGMDVWGQGTTVTVSS

SEQ ID NO: 106 - protein, 107 aa, artificial sequence
  P1-047310 VL
DIQMTQSPTSLSASVGDRVTITCRASQGISSWLAWYQQKPEKAPKSLIYAASSLQSGVPSRFSGSGSGTDYTLTISSLQPEDFATYYCQQYNSYPLTFGGGTKVEIK

SEQ ID NO: 107 - protein, 127 aa, artificial sequence
  P1-047299 VH
QVQLVQSGAEVKKPGSSVKVSCKASGGTFNRYAFSWVRQAPGQGLEWMGGIIPIFGTPNYAQKFQGRVTITADESTSTAYMELSSLISEDTAVYYCASSHFYGSGSSHFYYYGMHVWGQGTTVTVSS

SEQ ID NO: 108 - protein, 107 aa, artificial sequence
  P1-047299 VL
DIQMTQSPTSLSASVGDRVTITCRASQGISSWLAWYQQKPEKAPKSLIYAASSLQSGVPSRFSGSGSGTDFTLTISSLQPEDFATYYCQQYNSYPWTFGQGTKVEIK

SEQ ID NO: 109 - protein, 127 aa, artificial sequence
  P1-047301 VH
QVQLVQSGAEVKKPGSSVKVSCKASGGTFNRYAFSWVRQAPGQGLEWMGGIIPIFGTPNYAQKFQGRVTITADESTSTAYMELSSLISEDTAVYYCASSHFYGSGSSNYYYYGLDVWGQGTTVTVSS

SEQ ID NO: 110 - protein, 126 aa, artificial sequence
  P1-047289 VH
QVQLVQSGAEVKKPGSSVKVSCKASGGTFSSSAISWVRQAPGQGLEWMGGIIPIFGTADSAQKFQGRVTITADESTSTAYMELNSLRSEDTAVYYCAFTPRYRGSSHHYFYALGVWGQGTTVTVSS

SEQ ID NO: 111 - protein, 107 aa, artificial sequence
  P1-047289 VL
DIQMTQSPTSLSASVGDRVTITCRASQGISSWLAWYQQKPEKAPKSLIYAASSLQSGVPSRFSGSGSGTDFTLTISSLQPEDFATYYCQQFNSYPLTFGGGTKVEIK

SEQ ID NO: 112 - protein, 125 aa, artificial sequence
  P1-047306 VH
QVQLVQSGAEVKKPGSSVKVSCKASGGTFSSSAISWVRQAPGQGLEWMGGIIPIFGTANYAQKFQGRVTITADESTSTAYMELSSLRSEDTAVYYCARASQSRSSNYYYYGLDVWGQGTTVTVSS

SEQ ID NO: 113 - protein, 5 aa, artificial sequence
  P1-047305 HCDR1
SSAVS

SEQ ID NO: 114 - protein, 17 aa, artificial sequence
  P1-047305 HCDR2
GITPIFGTADYAQKFQG

SEQ ID NO: 115 - protein, 17 aa, artificial sequence
  P1-047305 HCDR3
TPRYRGSSHHYYYALGV

SEQ ID NO: 116 - protein, 11 aa, artificial sequence
  LCDR1
RASQGISSWLA

SEQ ID NO: 117 - protein, 9 aa, artificial sequence
  LCDR3
QQYNSYPYT

SEQ ID NO: 118 - protein, 5 aa, artificial sequence
  P1-047309 HCDR1
TYAIS

SEQ ID NO: 119 - protein, 17 aa, artificial sequence
  P1-047309 HCDR2
GINPIFGTANYAQKFQG

SEQ ID NO: 120 - protein, 8 aa, artificial sequence
  P1-047309 HCDR3
GGAVGFAY

SEQ ID NO: 121 - protein, 9 aa, artificial sequence
  P1-047309 LCDR3
QQANSFPFT

SEQ ID NO: 122 - protein, 5 aa, artificial sequence
  P1-047317 HCDR1
IYVIS

SEQ ID NO: 123 - protein, 17 aa, artificial sequence
  P1-047313 HCDR2
GIIPLFGTPNYAQRFQD

SEQ ID NO: 124 - protein, 16 aa, artificial sequence
  P1-047317 HCDR3
GHGPGSSHYSYYGLDV

SEQ ID NO: 125 - protein, 9 aa, artificial sequence
  P1-047299 LCDR3
QQYNSYPWT

SEQ ID NO: 126 - protein, 17 aa, artificial sequence
  P1-047317 HCDR2
GIIPLFGTPNYAQQFQD

SEQ ID NO: 127 - protein, 17 aa, artificial sequence
  P1-047311 HCDR2
GIIPLFGTANYAQQFQD

SEQ ID NO: 128 - protein, 5 aa, artificial sequence
  P1-047294, P1-047290, P1-047291, P1-047296, P1-047297, P1-047300, P1-047302 HCDR1
RHAIS

SEQ ID NO: 129 - protein, 17 aa, artificial sequence
  P1-047294 HCDR2
GIIPLFGTANYAQEFQG

SEQ ID NO: 130 - protein, 18 aa, artificial sequence
  P1-047294 HCDR3
SYFYGSGSSNYYYYGLDV

SEQ ID NO: 131 - protein, 17 aa, artificial sequence
  P1-047319 HCDR2
GIIPLFGTANYAQKFQG

SEQ ID NO: 132 - protein, 18 aa, artificial sequence
  P1-047302 HCDR3
SYFYGSGSSNYYYYGLDV

SEQ ID NO: 133 - protein, 9 aa, artificial sequence
  P1-047328 LCDR3
QQYNSYPIT

SEQ ID NO: 134 - protein, 17 aa, artificial sequence
  P1-047292 HCDR2
GIIPIFSTGNYAQKFQG

SEQ ID NO: 135 - protein, 17 aa, artificial sequence
  P1-047292 HCDR3
STRVRGVSHYYYYGLDV

SEQ ID NO: 136 - protein, 5 aa, artificial sequence
  P1-047322 HCDR1
SYAFT

SEQ ID NO: 137 - protein, 17 aa, artificial sequence
  P1-047322 HCDR2
GIIPIFRTANYAQKFQG

SEQ ID NO: 138 - protein, 18 aa, artificial sequence
  P1-047322 HCDR3
SHFSGSGSSHYYYYGMHV

SEQ ID NO: 139 - protein, 5 aa, artificial sequence
  P1-047310 HCDR1
RYAIS

SEQ ID NO: 140 - protein, 17 aa, artificial sequence
  P1-047310 HCDR2
GIIPIFGTSNYAQKFQG

SEQ ID NO: 141 - protein, 16 aa, artificial sequence
  P1-047310 HCDR3
GGNSWTTSLYYYGMDV

SEQ ID NO: 142 - protein, 5 aa, artificial sequence
  P1-047299, P1-047301 HCDR1
RYAFS

SEQ ID NO: 143 - protein, 17 aa, artificial sequence
  P1-047299, P1-047301 HCDR2
GIIPIFGTPNYAQKFQG

SEQ ID NO: 144 - protein, 18 aa, artificial sequence
  P1-047299 HCDR3
SHFYGSGSSHFYYYGMHV

SEQ ID NO: 145 - protein, 18 aa, artificial sequence
  P1-047301 HCDR3
SHFYGSGSSNYYYYGLDV

SEQ ID NO: 146 - protein, 17 aa, artificial sequence
  P1-047289 HCDR2
GIIPIFGTADSAQKFQG

SEQ ID NO: 147 - protein, 17 aa, artificial sequence
  P1-047289 HCDR3
TPRYRGSSHHYFYALGV

SEQ ID NO: 148 - protein, 9 aa, artificial sequence
  P1-047289 LCDR3
QQFNSYPLT

SEQ ID NO: 149 - protein, 17 aa, artificial sequence
  P1-047306 HCDR2
GIIPIFGTANYAQKFQG

SEQ ID NO: 150 - protein, 16 aa, artificial sequence
  P1-047306 HCDR3
ASQSRSSNYYYYGLDV

SEQ ID NO: 151 - protein, 5 aa, artificial sequence
  LSPGK (C-terminal end of heavy chain)
LSPGK

SEQ ID NO: 152 - protein, 4 aa, artificial sequence
  LSPG (C-terminal end of heavy chain)
LSPG

SEQ ID NO: 153 - protein, 126 aa, artificial sequence
  P1-047323, P1-047328 VH
QVQLVQSGAEVKKPGSSVKVSCKASGGTFINSEAINWVRQAPGQGLEWMGGIIPIFDITNYAQKFQGRVTITADESMSTAYMELSSLRSEDTAVYYCAKTYYDILTYHYHYGMDVWGQGTTVTVSS

SEQ ID NO: 154 - protein, 107 aa, artificial sequence
  P1-047323 VL
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIK

SEQ ID NO: 155 - protein, 107 aa, artificial sequence
  P1-047328 VL
AIQLTQSPSSLSASVGDRVTITCRASQGISSALAWYQQKPGKAPKLLIYDASSLESGVPSRFSGSGSGTDFTLTISSLQPEDFATYYCQQYNSYPITFGQGTRLEIK

SEQ ID NO: 156 - protein, 126 aa, artificial sequence
  P1-047263, P1-047265 VH
QVQLVQSGAEVKKPGASVKVSCKASGYTFPTYDINWVRQATGQGLEWMGWVNPNSGNTGYAQKFQDRVTMTRNTSISTAYMELSSLRSEDTAVYYCASDGLNMVRGVHNYYGMDVWGQGTTVTVSS

SEQ ID NO: 157 - protein, 107 aa, artificial sequence
  P1-047263 VL
DIQMTQSPTSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIK

SEQ ID NO: 158 - protein, 107 aa, artificial sequence
  P1-047317 VL
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIK

SEQ ID NO: 159 - protein, 6 aa, artificial sequence
  P1-047323, P1-047328 HCDR1
NSEAIN

SEQ ID NO: 160 - protein, 17 aa, artificial sequence
  P1-047323, P1-047328 HCDR2
GIIPIFDITNYAQKFQG

SEQ ID NO: 161 - protein, 16 aa, artificial sequence
  P1-047323, P1-047328 HCDR3
TYYDILTYHYHYGMDV

SEQ ID NO: 162 - protein, 9 aa, artificial sequence
  P1-047323 LCDR3
QQFNSYPIT

SEQ ID NO: 163 - protein, 5 aa, artificial sequence
  P1-047263, P1-047265 HCDR1
TYDIN

SEQ ID NO: 164 - protein, 17 aa, artificial sequence
  P1-047263, P1-047265 HCDR2
WVNPNSGNTGYAQKFQD

SEQ ID NO: 165 - protein, 17 aa, artificial sequence
  P1-047263, P1-047265 HCDR3
DGLNMVRGVHNYYGMDV

SEQ ID NO: 166 - protein, 9 aa, artificial sequence
  P1-047263 LCDR3
QQYNSYPPT

SEQ ID NO: 167 - protein, 11 aa, artificial sequence
  P1-047317 LCDR1
RASQSVSSYLA

SEQ ID NO: 168 - protein, 454 aa, artificial sequence
  P1-047239 HC
QVQLVESGGGVVQPGRSLRLSCAATEFTFSNYGMHWVRQAPGKGLEWVAVIWYDGSNKYYADSVKGRFTISRDNSKNTLYLQLNSLSAEDSAVYYCARDGRHYYGSTSYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 451 aa, artificial sequence
  P1-047246 HC
QVQLQESGPGLVKPSETLSLTCTVSGGSITNYYWTWIRQPPGKGLEWIGYIYDSGYTNYNPSLKSRVTLSIDTSKNQFSLKLSSVTAADTAVYYCARGVLWFGELLPLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - protein, 451 aa, artificial sequence; identical to SEQ ID NO: 169
  P1-047247 HC

SEQ ID NO: 171 - protein, 453 aa, artificial sequence
  P1-047248 HC
QVQLQESGPGLVKPSETLSLTCTVSGGSISSSYWSWVRQPPGKGLEWIGYTHYSGISNYNPSLKSRVTISVDTSKNQFSLKLSSVTAADTAVYYCAREGYDILTGYEYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 455 aa, artificial sequence
  P1-047323 HC
QVQLVQSGAEVKKPGSSVKVSCKASGGTFINSEAINWVRQAPGQGLEWMGGIIPIFDITNYAQKFQGRVTITADESMSTAYMELSSLRSEDTAVYYCAKTYYDILTYHYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 455 aa, artificial sequence; identical to SEQ ID NO: 172
  P1-047328 HC

SEQ ID NO: 174 - protein, 452 aa, artificial sequence
  P1-047334 HC
QVQLVQSGAEVKRPGSSVKVSCKASGGTFSSSAISWVRQAPGQGLEWMGGIIPIFGTTNGAQKFQGRVTITADESTSTAYMELSSLRSEDTAVYYCAAMVRGNYFYFYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 175 - protein, 455 aa, artificial sequence
  P1-047263 HC
QVQLVQSGAEVKKPGASVKVSCKASGYTFPTYDINWVRQATGQGLEWMGWVNPNSGNTGYAQKFQDRVTMTRNTSISTAYMELSSLRSEDTAVYYCASDGLNMVRGVHNYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 455 aa, artificial sequence; identical to SEQ ID NO: 175
  P1-047265 HC

SEQ ID NO: 177 - protein, 455 aa, artificial sequence
  P1-047289 HC
QVQLVQSGAEVKKPGSSVKVSCKASGGTFSSSAISWVRQAPGQGLEWMGGIIPIFGTADSAQKFQGRVTITADESTSTAYMELNSLRSEDTAVYYCAFTPRYRGSSHHYFYALGVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 178 - protein, 456 aa, artificial sequence
  P1-047290 HC
QVQLVQSGAEVKKPGSSVKVSCKASGGTFNRHAISWVRQAPGQGLEWMGGIIPLFGTANYAQKFQGRVTIAADEPTSTTYMELRSLRSEDTAVYYCASSYFYGSGSSNYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 456 aa, artificial sequence; identical to SEQ ID NO: 178
  P1-047291 HC

SEQ ID NO: 180 - protein, 455 aa, artificial sequence
  P1-047292 HC
QVQLVQSGAEVKKPGSSVKVSCKASGGTFSSSAISWVRQAPGQGLEWMGGIIPIFSTGNYAQKFQGRVTITADESTNTAYMDLSSLRSEDTAVYYCARSTRVRGVSHYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 181 - protein, 456 aa, artificial sequence
  P1-047294 HC
QVQLVQSGAEVKKPGSSVKVSCKASGGTFNRHAISWVRQAPGQGLEWMGGIIPLFGTANYAQEFQGRVTIAADEPTSTTYMELRSLRSEDTAVYYCASSYFYGSGSSNYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 456 aa, artificial sequence; identical to SEQ ID NO: 178
  P1-047296 HC

SEQ ID NO: 183 - protein, 456 aa, artificial sequence; identical to SEQ ID NO: 178
  P1-047297 HC

SEQ ID NO: 184 - protein, 456 aa, artificial sequence
  P1-047299 HC
QVQLVQSGAEVKKPGSSVKVSCKASGGTFNRYAFSWVRQAPGQGLEWMGGIIPIFGTPNYAQKFQGRVTITADESTSTAYMELSSLISEDTAVYYCASSHFYGSGSSHFYYYGMHVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 185 - protein, 456 aa, artificial sequence; identical to SEQ ID NO: 178
  P1-047300 HC

SEQ ID NO: 186 - protein, 456 aa, artificial sequence
  P1-047301 HC
QVQLVQSGAEVKKPGSSVKVSCKASGGTFNRYAFSWVRQAPGQGLEWMGGIIPIFGTPNYAQKFQGRVTITADESTSTAYMELSSLISEDTAVYYCASSHFYGSGSSNYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 456 aa, artificial sequence; identical to SEQ ID NO: 178
  P1-047302 HC

SEQ ID NO: 188 - protein, 455 aa, artificial sequence
  P1-047305 HC
QVQLVQSGAEVKKPGSSVKVSCKTSGGTFSSSAVSWVRQAPGQGLEWMGGITPIFGTADYAQKFQGRVTITADASTSTGYMELSSLRSEDTAVYYCAFTPRYRGSSHHYYYALGVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 454 aa, artificial sequence
  P1-047306 HC
QVQLVQSGAEVKKPGSSVKVSCKASGGTFSSSAISWVRQAPGQGLEWMGGIIPIFGTANYAQKFQGRVTITADESTSTAYMELSSLRSEDTAVYYCARASQSRSSNYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 454 aa, artificial sequence
  P1-047307 HC
QVQLVQSGAEVKRPGSSVKVSCKASGGTFSIYVISWVRQAPGQGLEWMGGIIPLFGTPNYAQQFQDRVTITADESTRTAYMELNSLKSEDTAVYYCARGHGPGSSHYSYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 191 - protein, 454 aa, artificial sequence
  P1-047308 HC
QVQLVQSGAEVKRPGSSVKVSCKASGGTFSIYVISWVRQAPGQGLEWMGGIIPLFGTANYAQKFQGRVTITADESTNTAYMELSSLRSEDTAVYYCARGHGPGSSHYSYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 446 aa, artificial sequence
  P1-047309 HC
QVQLVQSGAEVKKPGSSVKVSCNPSGGTFSTYAISWVRQAPGQGLEWMGGINPIFGTANYAQKFQGRVTITADESTSPGYLELSSLRSEDTAVYYCARGGAVGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 193 - protein, 454 aa, artificial sequence
  P1-047310 HC
QVQLVQSGAEVKKPGSSVKVSCKASGGTFSRYAISWVRQAPGQGLEWMGGIIPIFGTSNYAQKFQGRVTIKADESTSTAYMELSSLRSEDTAVYYCARGGNSWTTSLYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 194 - protein, 454 aa, artificial sequence
  P1-047311 HC
QVQLVQSGAEVKKPGSSVKVSCKASGGTFSIYVISWVRQAPGQGLEWMGGIIPLFGTANYAQQFQDRVTITADESTRTAYMELNSLKSEDTAVYYCARGHGPGSSHYSYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 195 - protein, 454 aa, artificial sequence; identical to SEQ ID NO: 190
  P1-047312 HC

SEQ ID NO: 196 - protein, 454 aa, artificial sequence
  P1-047313 HC
QVQLVQSGAEVKRPGSSVKVSCKASGGTFSIYVISWVRQAPGQGLEWMGGIIPLFGTPNYAQRFQDRVTITADESTRTAYMELSSLRSEDTAVYYCARGHGPGSSHYSYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 197 - protein, 454 aa, artificial sequence; identical to SEQ ID NO: 190
  P1-047314 HC

SEQ ID NO: 198 - protein, 454 aa, artificial sequence; identical to SEQ ID NO: 190
  P1-047317 HC

SEQ ID NO: 199 - protein, 454 aa, artificial sequence
  P1-047318 HC
QVQLVQSGAEVKRPGSSVKVSCKASGGTFSIYVISWVRQAPGQGLEWMGGIIPLFGTPNYAQQFQDRVTITADESTRTAYMELSSLRSEDTAVYYCARGHGPGSSHYSYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 200 - protein, 454 aa, artificial sequence; identical to SEQ ID NO: 191
  P1-047319 HC

SEQ ID NO: 201 - protein, 454 aa, artificial sequence; identical to SEQ ID NO: 190
  P1-047320 HC

SEQ ID NO: 202 - protein, 456 aa, artificial sequence
  P1-047322 HC
QVQLVQSGAEVKKPGSSVKVSCKSSGGTFSSYAFTWVRQAPGQGLEWMGGIIPIFRTANYAQKFQGRVTITADESTSTAYMELSSLRSEDTAVYYCASSHFSGSGSSHYYYYGMHVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 203 - protein, 214 aa, artificial sequence
  P1-047239, P1-047246 LC
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 215 aa, artificial sequence
  P1-047247 LC
EIVLTQSPGTLSLSPGERATLSCRASQSVSSSYLAWYQQKPGQAPRLLIYGASSRATGIPERFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 214 aa, artificial sequence
  P1-047248 LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 214 aa, artificial sequence
  P1-047323 LC
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 214 aa, artificial sequence
  P1-047328 LC
AIQLTQSPSSLSASVGDRVTITCRASQGISSALAWYQQKPGKAPKLLIYDASSLESGVPSRFSGSGSGTDFTLTISSLQPEDFATYYCQQYNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - protein, 214 aa, artificial sequence
  P1-047334 LC
AIQLTQSPSSLSASVGDRVTITCRASQGISSALAWYQQKPGKAPKLLIYAASSLQSGVPSRFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 214 aa, artificial sequence
  P1-047263 LC
DIQMTQSPTSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 214 aa, artificial sequence
  P1-047265, P1-047290, P1-047292, P1-047301, P1-047302, P1-047305, P1-047306, P1-047308, P1-047314, P1-047322 LC
DIQMTQSPTSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 214 aa, artificial sequence
  P1-047289 LC
DIQMTQSPTSLSASVGDRVTITCRASQGISSWLAWYQQKPEKAPKSLIYAASSLQSGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 214 aa, artificial sequence
  P1-047291 LC
DIQMTQSPTSLSASVGDRVTITCRASQGISSWLAWYQQKPEKAPKSLIYAASSLQSGVPSRFSGSGSGTDFTLTISSLQPDDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 214 aa, artificial sequence
  P1-047294 LC
DIQMTQSPTSLSASVGDRVTITCRASQGISSWLAWYQQKPEKAPKSLIYAASSLQSGVPSRFSGGGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 214 aa, artificial sequence
  P1-047296 LC
DIQMTQSPTSLSASVGDRVTITCRASQGISSWLAWYQQKPEKAPKSLIYAASSLQSGVPSRFSGSGSGTDFTLTISSLQPEDFATYYCQQYNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 214 aa, artificial sequence
  P1-047297 LC
DIQMTQSPTSLSASVGDRVTITCRASQGISSWLAWYQHKPGKAPKSLIYAASSLQSGVPSRFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 214 aa, artificial sequence
  P1-047299 LC
DIQMTQSPTSLSASVGDRVTITCRASQGISSWLAWYQQKPEKAPKSLIYAASSLQSGVPSRFSGSGSGTDFTLTISSLQPEDFATYYCQQYNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 214 aa, artificial sequence
  P1-047300 LC
DIQMTQSPTSLSASVGDRVTITCRASQGISSWLAWYQQKPEKAPKSLIHAASSLQSGVPSRFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 214 aa, artificial sequence
  P1-047307 LC
DIQMTQSPTSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 214 aa, artificial sequence
  P1-047309 LC
DIQMTQSPSSVSASVGDRVTITCRASQGISSWLAWYQHKPGKAPKLLIYAASSLQSGVPSRFSGSGSGTDFTLTISSLQPEDFATYYCQQANSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 214 aa, artificial sequence
  P1-047310 LC
DIQMTQSPTSLSASVGDRVTITCRASQGISSWLAWYQQKPEKAPKSLIYAASSLQSGVPSRFSGSGSGTDYTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 214 aa, artificial sequence
  P1-047311 LC
DIQMTQSPTSLSASVGDRVTITCRASQGISSWLAWYQQKPGKAPKSLIYAASSLQSGVPSRFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 214 aa, artificial sequence
  P1-047312, P1-047318 LC
DIQMTQSPTSLSASVGDRVTITCRASQGISSWLAWYQHKPGKAPKLLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 214 aa, artificial sequence
  P1-047313 LC
DIQMTQSPTSLSASVGDRVTITCRASQGISSWLAWYQHKPGKAPKLLIYAASSLQSGVPSRFSGSGSGTDFTLTISSLQPEDFATYYCQQYNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 214 aa, artificial sequence
  P1-047317 LC
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 214 aa, artificial sequence
  P1-047319, P1-047320 LC
DIQMTQSPTSLSASVGDRVTITCRASQGISSWLAWYQHKPGKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - DNA, 1362 bp, artificial sequence
  P1-047239 HC
caggtgcagctggtggagtctgggggaggcgtggtccagcctgggaggtccctgagactctcctgtgcagctactgaattcaccttcagtaactatggcatgcactgggtccgccaggctccaggcaaggggctggagtgggtggcagttatatggtatgatggaagtaataaatactatgcagactccgtgaagggccgcttcaccatctccagagacaattccaagaacacgctgtatctgcaattgaacagcctgagcgccgaggactcggctgtgtattactgtgcgagagatgggaggcattactatggttcgacctcctacttcggcatggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 227 - DNA, 1353 bp, artificial sequence
  P1-047246 HC
caggtgcagctgcaggagtcgggcccaggactggtgaagccttcggagaccctgtccctcacctgcactgtctctggtggctccatcactaattactactggacctggatccggcagcccccagggaagggactggagtggattgggtatatctatgacagtgggtacaccaactacaacccctccctcaagagtcgagtcaccttatcaatagacacgtccaagaaccagttctccctgaagctgagctctgtgaccgctgcggacacggccgtttattactgtgcgagaggggttctatggttcggggagttattacctctccttgactactggggccagggaaccctggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 228 - DNA, 1353 bp, artificial sequence; identical to SEQ ID NO: 227
  P1-047247 HC

SEQ ID NO: 229 - DNA, 1359 bp, artificial sequence
  P1-047248 HC
caggtgcagctgcaggagtcgggcccaggactggtgaagccttcggagaccctgtccctcacctgcactgtgtctggtggctccatcagtagttcctactggagctgggtccggcagcccccagggaagggactggagtggattggatatacccattacagtgggatcagcaactacaacccctccctcaagagtcgagtcaccatatcagtagacacgtccaagaaccagttctccctgaagctgagctctgtgaccgctgcggacacggccgtgtattactgtgcgagagaagggtacgatattttgactggttatgagtactacggtatggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 230 - DNA, 1365 bp, artificial sequence
  P1-047323 HC
caggtgcagctggtgcagtctggggctgaggtgaagaagcctgggtcctcggtgaaggtctcctgcaaggcttctggaggcaccttcatcaatagcgaagctatcaactgggtgcgacaggcccctggacaagggcttgagtggatgggagggatcatccctatctttgacattacaaactacgcacagaagttccagggcagagtcacgattaccgcggacgaatccatgagcacagcctacatggagctgagcagcctgagatctgaggacacggccgtgtattactgtgcgaagacgtattacgatattttgacttatcactatcactacggtatggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 231 - DNA, 1365 bp, artificial sequence; identical to SEQ ID NO: 230
  P1-047328 HC

SEQ ID NO: 232 - DNA, 1356 bp, artificial sequence
  P1-047334 HC
caggtgcagctggtgcagtctggggctgaggtgaagaggcctgggtcctctgtgaaggtctcctgcaaggcttctggaggcaccttcagtagttccgctatcagctgggtgcgacaggcccctggacaagggcttgagtggatgggagggatcatccctatctttggtacaacaaacggcgcacagaagttccagggcagagtcacgattaccgcggacgaatccacgagcacagcctacatggagctgagcagcctgagatctgaggacacggccgtgtattactgtgcggctatggttcggggaaattacttctacttctacggtatggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 233 - DNA, 1365 bp, artificial sequence
  P1-047263 HC
caggtgcagctggtgcagtctggggctgaggtgaagaagcctggggcctcagtgaaggtctcctgcaaggcttctggatacaccttccccacttatgatatcaactgggtgcgacaggccactggacaagggcttgagtggatgggatgggtgaaccctaacagtggtaacacaggctatgcacagaagttccaggacagagtcaccatgaccaggaacacctccataagcacagcctacatggagctgagcagcctgagatctgaggacacggccgtgtattactgtgcgagtgacggccttaatatggttcggggagttcacaactactacggtatggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 234 - DNA, 1365 bp, artificial sequence; identical to SEQ ID NO: 233
  P1-047265 HC

SEQ ID NO: 235 - DNA, 1365 bp, artificial sequence
  P1-047289 HC
caggtgcagctggtgcagtctggggctgaggtgaagaagcctgggtcctcggtgaaggtctcctgcaaggcttctggaggcaccttcagcagctctgctatcagctgggtgcgacaggcccctggacaagggcttgagtggatgggaggaatcatccctatcttcggtacagcagactccgcacagaagttccagggcagagtcacgattaccgcggacgagtccacgagcacagcctacatggaattgaacagcctgagatctgaggacacggccgtttactactgtgcgttcacaccccggtatcgtgggagctcccaccactacttctacgctttgggcgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 236 - DNA, 1368 bp, artificial sequence
  P1-047290 HC
caggtgcagctggtgcagtctggggctgaggtgaagaagcctgggtcctcggtgaaggtctcctgcaaggcttctggaggcaccttcaacagacatgctatcagctgggtgcgacaggcccctggacaagggcttgagtggatgggagggatcatccctctctttggtacagcaaactacgcgcagaagttccagggcagagtcacgattgccgcggacgaacccacgagcacaacctacatggagctgcgcagcctgagatctgaggacacggccgtgtattactgtgcgagttcgtatttctatggttcggggagttccaactactactactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 237 - DNA, 1368 bp, artificial sequence; identical to SEQ ID NO: 236
  P1-047291 HC

SEQ ID NO: 238 - DNA, 1365 bp, artificial sequence
  P1-047292 HC
caggtgcagctggtgcagtctggggctgaggtgaagaagcctgggtcctcggtgaaggtctcctgcaaggcttctggaggcaccttcagcagctctgctatcagctgggtgcgacaggcccctggacaagggcttgagtggatgggagggatcatccctatctttagtacaggaaactacgcacagaagttccagggcagagtcacgattaccgcggacgaatccacgaacacagcctacatggatctgagcagcctgagatctgaggacacggccgtgtattactgtgcgagatccactagggttcggggagtttcccactactactactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 239 - DNA, 1368 bp, artificial sequence
  P1-047294 HC
caggtgcagctggtgcagtctggggctgaggtgaagaagcctgggtcctcggtgaaggtctcctgcaaggcttctggaggcaccttcaacagacatgctatcagctgggtgcgacaggcccctggacaagggcttgagtggatgggagggatcatccctctctttggtacagcaaactacgcgcaggagttccagggcagagtcacgattgccgcggacgaacccacgagcacaacctacatggagctgcgcagcctgagatctgaggacacggccgtgtattactgtgcgagttcgtatttctatggttcggggagttccaactactactactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 240 - DNA, 1368 bp, artificial sequence; identical to SEQ ID NO: 236
  P1-047296 HC

SEQ ID NO: 241 - DNA, 1368 bp, artificial sequence; identical to SEQ ID NO: 236
  P1-047297 HC

SEQ ID NO: 242 - DNA, 1368 bp, artificial sequence
  P1-047299 HC
caggtgcagctggtgcagtctggggctgaggtgaagaagcctgggtcctcggtgaaggtctcctgcaaggcttctggaggcaccttcaacagatatgctttcagctgggtgcgacaggcccctggacaagggcttgagtggatgggagggatcatccctatctttggtacacccaactacgcacagaagttccagggcagagtcacgattaccgcggacgaatccacgagcacggcctacatggagctgagcagcctgatatctgaggacacggccgtgtattactgtgcgagcagccatttctatggttcggggagttcccacttttactactacggtatgcacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 243 - DNA, 1368 bp, artificial sequence
  P1-047300 HC
caggtgcagctggtgcagtctggggctgaggtgaagaagcctgggtcctcggtgaaggtctcctgcaaggcttctggaggcaccttcaacagacatgctatcagctgggtgcgacaggcccctggacaagggcttgagtggatgggagggatcatccctctctttggtacagcaaactacgcgcagaagttccagggcagagtcacgattgccgcggacgaacccacgagcacaacctacatggagctgcgcagcctgagatctgaggacacggccgtgtattactgtgcaagttcgtatttctatggttcggggagttccaactactactactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 244 - DNA, 1368 bp, artificial sequence
  P1-047301 HC
caggtgcagctggtgcagtctggggctgaggtgaagaagcctgggtcctcggtgaaggtctcctgcaaggcttctggaggcaccttcaacagatatgctttcagctgggtgcgacaggcccctggacaagggcttgagtggatgggagggatcatccctatctttggtacacccaactacgcacagaagttccagggcagagtcacgattaccgcggacgaatccacgagcacggcctacatggagctgagcagcctgatatctgaggacacggccgtgtattactgtgcgagcagccatttctatggttcggggagttccaactactactactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 245 - DNA, 1368 bp, artificial sequence
  P1-047302 HC
caggtgcagctggtgcagtctggggctgaggtgaagaagcctgggtcctcggtgaaggtctcctgcaaggcttctggaggcaccttcaacagacatgctatcagctgggtgcgacaggcccctggacaagggcttgagtggatgggagggatcatccctctctttggtacagcaaactacgcgcagaagttccagggcagagtcacgattgccgcggacgaacccacgagcacaacctacatggagctgcgcagcctgagatctgaggacacggccgtgtattactgtgcgagttcgtatttctatggttcggggagttccaactattactactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 246 - DNA, 1365 bp, artificial sequence
  P1-047305 HC
caggtgcagctggtgcagtctggggctgaggtgaagaagcctgggtcctcggtgaaggtctcctgcaagacttctggaggcaccttcagcagctctgctgtcagctgggtgcgacaggcccctggacaagggcttgaatggatgggaggaatcacccctatttttggtacagcagactacgcacagaagttccagggcagagtcacgattaccgcggacgcatccacgagcacaggttatatggaactgagcagcctgagatctgaggacacggccgtttactactgtgcgttcacaccccgatatcgtgggagctcccaccactactactacgctttgggcgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 247 - DNA, 1362 bp, artificial sequence
  P1-047306 HC
caggtgcagctggtgcagtctggggctgaggtgaagaagcctgggtcctcggtgaaggtctcctgcaaggcttctggaggcaccttcagcagctctgctatcagctgggtacgacaggcccctggacaagggcttgagtggatgggagggatcatccctatctttggtacagcgaactacgcacagaagttccagggcagagtcacgattaccgcggacgaatccacgagcacagcctacatggagctgagcagcctgagatctgaggacacggccgtgtattactgtgcgagagcctcccaaagcaggagctcgaactactactactacggtctggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 248 - DNA, 1362 bp, artificial sequence
  P1-047307 HC
caggtgcagctggtgcagtctggggctgaggtgaagaggcctgggtcctcggtgaaggtctcctgcaaggcttctggaggcaccttcagtatctatgttatcagctgggtgcgacaggcccctggacaagggcttgagtggatgggagggatcatccctctctttggtacaccaaactacgcacagcagttccaggacagagtcacgattaccgcggacgaatccacgaggacagcctacatggagctgaatagcctgaaatctgaggacacggccgtatattactgtgcgaggggacatggtccggggagttcccactactcctactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 249 - DNA, 1362 bp, artificial sequence
  P1-047308 HC
caggtgcagctggtgcagtctggggctgaggtgaagaggcctgggtcctcggtgaaggtctcctgcaaggcttctggaggcaccttcagtatctatgttatcagctgggtgcgacaggcccctggacaagggcttgagtggatgggagggatcatccctctctttggtacagcaaactacgcacagaagttccagggcagagtcacgattaccgcggacgaatccacgaatacagcctacatggagctgagtagcctgagatctgaggacacggccgtgtattactgtgcgaggggacatggtccggggagttcccactactcctactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 250 - DNA, 1338 bp, artificial sequence
  P1-047309 HC
caggtgcagctggtgcagtctggggctgaggtgaagaagcctgggtcctcggtgaaggtctcctgcaacccttctggaggcaccttcagcacctacgctatcagctgggtgcgacaggcccctggacaagggcttgagtggatgggagggatcaaccctatctttggaacagcaaactacgcacagaagttccagggcagagtcacaattaccgcggacgaatccacgagtccaggctacctggagctgagcagcctgagatctgaggacacggccgtttattactgtgcgagagggggagcagtgggttttgcctattggggccagggaaccctggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 251 - DNA, 1362 bp, artificial sequence
  P1-047310 HC
caggtgcagctggtgcagtctggggctgaggtgaagaagcctgggtcctcggtgaaggtctcctgcaaggcttctggaggcaccttcagcagatatgctatcagctgggtgcgacaggcccctggacaagggcttgagtggatgggagggatcatccctatctttggtacatcaaactacgcacagaagttccagggcagagtcacgattaaagcggacgaatccacgagcacagcctacatggagctgagcagcctgagatctgaggacacggccgtgtattactgtgcgagagggggcaacagctggaccactagtttgtactactacggtatggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 252 - DNA, 1362 bp, artificial sequence
  P1-047311 HC
caggtgcagctggtgcagtctggggctgaggtgaagaagcctgggtcctcggtgaaggtctcctgcaaggcttctggaggcaccttcagtatctatgttatcagctgggtgcgacaggcccctggacaagggcttgagtggatgggagggatcatccctctctttggtacagcaaactacgcacagcagttccaggacagagtcacgattaccgcggacgaatccacgaggacagcctacatggagctgaatagcctgaaatctgaggacacggctgtatattactgtgcgaggggacatggtccggggagttcccactactcctactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 253 - DNA, 1362 bp, artificial sequence; identical to SEQ ID NO: 248
  P1-047312 HC

SEQ ID NO: 254 - DNA, 1362 bp, artificial sequence
  P1-047313 HC
caggtgcagctggtgcagtctggggctgaggtgaagaggcctgggtcctcggtgaaggtctcctgcaaggcttctggaggcaccttcagtatctatgttatcagctgggtgcgacaggcccctggacaagggcttgagtggatgggagggatcatccctctctttggtacaccaaactacgcacagcggttccaggacagagtcacgattaccgcggacgaatccacgaggacagcctacatggagctgagtagcctgagatctgaggacacggccgtgtattactgtgcgaggggacatggtccggggagttcccactactcctactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 255 - DNA, 1362 bp, artificial sequence; identical to SEQ ID NO: 248
  P1-047314 HC

SEQ ID NO: 256 - DNA, 1362 bp, artificial sequence; identical to SEQ ID NO: 248
  P1-047317 HC

SEQ ID NO: 257 - DNA, 1362 bp, artificial sequence
  P1-047318 HC
caggtgcagctggtgcagtctggggctgaggtgaagaggcctgggtcctcggtgaaggtctcctgcaaggcttctggaggcaccttcagtatctatgttatcagctgggtccgacaggcccctggacaagggcttgagtggatgggagggatcatccctctctttggtacaccaaactacgcacagcagttccaggacagagtcacgattaccgcggacgaatccacgaggacagcctacatggagctgagtagcctgagatctgaggacacggccgtgtattactgtgcgaggggacatggtccggggagttcccactactcctactacggtttggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 258 - DNA, 1362 bp, artificial sequence; identical to SEQ ID NO: 249
  P1-047319 HC

SEQ ID NO: 259 - DNA, 1362 bp, artificial sequence; identical to SEQ ID NO: 248
  P1-047320 HC

SEQ ID NO: 260 - DNA, 1368 bp, artificial sequence
  P1-047322 HC
caggtgcagctggtgcagtctggggctgaggtgaagaagcctgggtcctcggtgaaggtctcctgcaagtcttctggaggcaccttcagcagctatgctttcacctgggtgcgacaggcccctggacaagggcttgagtggatgggagggatcatccctatctttcgtacagcaaactacgcacagaagttccagggcagagtcacgattaccgcggacgaatccacgagcacagcctacatggagctgagcagcctgagatctgaggacacggccgtgtattactgtgcgagcagccatttctctggttcgggaagttcccactactactactacggtatgcacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 261 - DNA, 642 bp, artificial sequence
  P1-047239 LC
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 262 - DNA, 642 bp, artificial sequence; identical to SEQ ID NO: 261
  P1-047246 LC

SEQ ID NO: 263 - DNA, 645 bp, artificial sequence
  P1-047247 LC
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagagaggttcagtggcagtgggtctgggacagacttcactctcaccatcagcagactggagcctgaagattttgcagtgtattactgtcagcagtatggtagctcaccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 264 - DNA, 642 bp, artificial sequence
  P1-047248 LC
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 265 - DNA, 642 bp, artificial sequence
  P1-047323 LC
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 266 - DNA, 642 bp, artificial sequence
  P1-047328 LC
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgccaacagtataatagttacccgatcaccttcggccaagggacacgactggagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 267 - DNA, 642 bp, artificial sequence
  P1-047334 LC
gccatccagttgacccagtctccatcctccctgtctgcatctgtaggagacagagtcaccatcacttgccgggcaagtcagggcattagcagtgctttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 268 - DNA, 642 bp, artificial sequence
  P1-047263 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctccgacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 269 - DNA, 642 bp, artificial sequence
  P1-047265 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 270 - DNA, 642 bp, artificial sequence
  P1-047289 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgtcaacagtttaatagttaccctctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 271 - DNA, 642 bp, artificial sequence; identical to SEQ ID NO: 269
  P1-047290 LC

SEQ ID NO: 272 - DNA, 642 bp, artificial sequence
  P1-047291 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgatgattttgcaacttattactgccaacagtataatagttacccgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 273 - DNA, 642 bp, artificial sequence; identical to SEQ ID NO: 269
  P1-047292 LC

SEQ ID NO: 274 - DNA, 642 bp, artificial sequence
  P1-047294 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcggtggatctgggacagatttcactctcaccatcagcagcctgcagcctgaagattttgcaacttattactgccaacagtataatagttacccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 275 - DNA, 642 bp, artificial sequence
  P1-047296 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgatcaccttcggccaagggacacgactggagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 276 - DNA, 642 bp, artificial sequence
  P1-047297 LC
gacatccagatgacccagtctccaacctcactgtctgcatctgtaggagacagagtcaccatcacttgtcgggcgagtcagggtattagcagctggttagcctggtatcagcataaaccagggaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 277 - DNA, 642 bp, artificial sequence
  P1-047299 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgtggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 278 - DNA, 642 bp, artificial sequence
  P1-047300 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatccatgctgcatccagtttgcaaagtggggtcccatcaaggttcagcggcagtggatctgggacagatttcactctcaccatcagcagcctgcagcctgaagattttgcaacttattactgccaacagtataatagttacccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 279 - DNA, 642 bp, artificial sequence; identical to SEQ ID NO: 269
  P1-047301 LC

SEQ ID NO: 280 - DNA, 642 bp, artificial sequence
  P1-047302 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 281 - DNA, 642 bp, artificial sequence; identical to SEQ ID NO: 269
  P1-047305 LC

SEQ ID NO: 282 - DNA, 642 bp, artificial sequence; identical to SEQ ID NO: 280
  P1-047306 LC

SEQ ID NO: 283 - DNA, 642 bp, artificial sequence; identical to SEQ ID NO: 280
  P1-047307 LC

SEQ ID NO: 284 - DNA, 642 bp, artificial sequence; identical to SEQ ID NO: 269
  P1-047308 LC

SEQ ID NO: 285 - DNA, 642 bp, artificial sequence
  P1-047309 LC
gacatccagatgacccagtctccatcttccgtgtctgcatctgtaggagacagagtcaccatcacttgtcgggcgagtcagggtattagcagctggttagcctggtatcagcataaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaatagtttcccgttcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 286 - DNA, 642 bp, artificial sequence
  P1-047310 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacggattacactctcaccatcagcagcctgcagcctgaagattttgcaacttattactgccaacagtataatagttacccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 287 - DNA, 642 bp, artificial sequence
  P1-047311 LC
gacatccagatgacccagtctccaacctcactgtctgcatctgtaggagacagagtcaccatcacttgtcgggcgagtcagggtattagcagctggttagcctggtatcagcagaaaccagggaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 288 - DNA, 642 bp, artificial sequence
  P1-047312 LC
gacatccagatgacccagtctccaacctcactgtctgcatctgtaggagacagagtcaccatcacttgtcgggcgagtcagggtattagcagctggttagcctggtatcagcataaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 289 - DNA, 642 bp, artificial sequence
  P1-047313 LC
gacatccagatgacccagtctccaacctcactgtctgcatctgtaggagacagagtcaccatcacttgtcgggcgagtcagggtattagcagctggttagcctggtatcagcataaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgccaacagtataatagttacccctggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 290 - DNA, 642 bp, artificial sequence; identical to SEQ ID NO: 269
  P1-047314 LC

SEQ ID NO: 291 - DNA, 642 bp, artificial sequence
  P1-047317 LC
gaaattgtgttgacgcagtctccaggcaccctgtctttgtctccaggggaaagagccaccctctcctgcagggccagtcagagtgttagcagctacttagcctggtaccaacagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 292 - DNA, 642 bp, artificial sequence; identical to SEQ ID NO: 288
  P1-047318 LC

SEQ ID NO: 293 - DNA, 642 bp, artificial sequence
  P1-047319 LC
gacatccagatgacccagtctccaacctcactgtctgcatctgtaggagacagagtcaccatcacttgtcgggcgagtcagggtattagcagctggttagcctggtatcagcataaaccagggaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 294 - DNA, 642 bp, artificial sequence; identical to SEQ ID NO: 293
  P1-047320 LC

SEQ ID NO: 295 - DNA, 642 bp, artificial sequence
  P1-047322 LC
gacatccagatgacccagtctccaacctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggtttagcggcagtggatctgggacagatttcactctcaccatcagcagcctgcagcctgaagattttgcaacttattactgccaacagtataatagttacccgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt